(19) United States
(12) Patent Application Publication
(10) Pub. No.: US 2003/0069296 A1
(43) Pub. Date: Apr. 10, 2003

(54) Substituted-aryl 7-aza[2.2.1]bicycloheptanes for the treatment of disease

(76) Inventors: Donn G. Wishka, Kalamazoo, MI (US); Jason K. Myers, Kalamazoo, MI (US); Vincent E. Groppi JR., Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/218,593

(22) Filed: Aug. 14, 2002

Related U.S. Application Data
(60) Provisional application No. 60/314,767, filed on Aug. 24, 2001.
(60) Provisional application No. 60/314,769, filed on Aug. 24, 2001.
(60) Provisional application No. 60/314,862, filed on Aug. 24, 2001.
(60) Provisional application No. 60/314,771, filed on Aug. 24, 2001.
(60) Provisional application No. 60/389,109, filed on Jun. 14, 2002.

(51) Int. Cl. A61K 31/403; C07D 487/02

(52) U.S. Cl. 514/412; 548/453

(57) ABSTRACT

The invention provides compounds of Formula I:

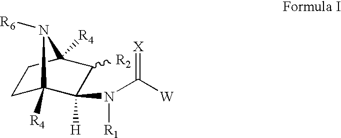

wherein the stereochemistry of the of the 7-azabicyclo[2.2.1]heptane ring is 1S, 2R, 4R and the nitrogen substituent at the C-2 carbon has the exo orientation; W is -Q, —C═C-Q, or —C≡C-Q; and Q is as defined herein. These compounds may be in the form of pharmaceutical salts or compositions, and are useful in pharmaceuticals used to treat diseases or conditions in which α7 is known to be involved.

### CROSS-REFERENCE TO RELATED APPLICATIONS

This application claims the benefit of U.S. provisional application Serial No. 60/314767 filed on Aug. 24, 2001, under 35 USC 119(e)(i) and U.S. provisional application Serial No. 60/314769 filed on Aug. 24, 200, under 35 USC 119(e)(i), U.S. provisional application Serial No. 60/314862 filed on Aug. 24, 2001, under 35 USC 119(e)(i), U.S. provisional application Serial No. 60/314771 filed on Aug. 24, 2001, under 35 USC 119(e)(i), and U.S. provisional No. 60/389109 filed Jun. 14, 2002, which are incorporated herein by reference in their entirety.

### FIELD OF INVENTION

Nicotinic acetylcholine receptors (nAChRs) play a large role in central nervous system (CNS) activity. Particularly, they are known to be involved in cognition, learning, mood, emotion, and neuroprotection. There are several types of nicotinic acetylcholine receptors, and each one appears to have a different role in regulating CNS function. Nicotine affects all such receptors, and has a variety of activities. Unfortunately, not all of the activities are desirable. In fact, one of the least desirable properties of nicotine is its addictive nature and the low ratio between efficacy and safety. The present invention relates to molecules that have a greater effect upon the α7 nAChRs as compared to other closely related members of this large ligand-gated receptor family. Thus, the invention provides compounds that are active drug molecules with fewer side effects.

### BACKGROUND OF THE INVENTION

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, Nature, 366(6454), p. 360-4, 1997). Another feature that makes functional assays of α7 nAChR challenging is that the receptor is rapidly (100 milliseconds) inactivated. This rapid inactivation greatly limits the functional assays that can be used to measure channel activity.

Recently, Eisele et al. has indicated that a chimeric receptor formed between the N-terminal ligand binding domain of the α7 nAChR (Eisele et al., Nature, 366(6454), p 479-83, 1993), and the pore forming C-terminal domain of the 5-HT₃ receptor expressed well in Xenopus oocytes while retaining nicotinic agonist sensitivity. Eisele et al. used the N-terminus of the avian (chick) form of the α7 nAChR receptor and the C-terminus of the mouse form of the 5-HT₃ gene. However, under physiological conditions the α7 nAChR is a calcium channel while the 5-HT₃R is a sodium and potassium channel. Indeed, Eisele et al. teaches that the chicken α7 nAChR/mouse 5-HT₃R behaves quite differently than the native α7 nAChR with the pore element not conducting calcium but actually being blocked by calcium ions. WO 00/73431 A2 reports on assay conditions under which the 5-HT₃R can be made to conduct calcium. This assay may be used to screen for agonist activity at this receptor.

U.S. Pat. No. 6,255,490 discloses7-azabicyclo[2.2.1]-heptane and -heptene derivatives as cholinergic receptor ligands.

U.S. Pat. No. 6,117,889 discloses discloses7-azabicyclo[2.2.1]-heptane and -heptene derivatives as analgesics and anti-inflammatory agents.

U.S. Pat. No. 6,060,473 discloses7-azabicyclo[2.2.1]-heptane and -heptene derivatives as cholinergic receptor ligands.

U.S. Pat. No. 6,054,464 discloses azabicyclic esters of carbamic acids useful in therapy, especially in the treatment or prophylaxis of psychotic disorders and intellectual impairment disorders, as well as intermediates and use of intermediates in synthesis.

U.S. Pat. No. 5,977,144 discloses compositions for benzylidene- and cinnamylidene-anabaseines and methods for using these compositions for treating conditions associated with defects or malfunctioning of nicotinic subtypes brain receptors. These compositions target the α7 receptor subtype with little or no activation of the (α4β2 or other receptor subtypes.

U.S. Pat. No. 5,919,793 discloses heterocyclic derivatives useful in lowering cholesterol levels in blood plasma.

U.S. Pat. No. 5,741,819 discloses arylsulfonylbenzene derivatives and their use as factor Xa inhibitors as being useful for the treatment of arterial and venous thrombotic occlusive disorders, inflammation, cancer, and neurodegenerative diseases.

U.S. Pat. No. 5,723,103 discloses substituted benzamides and radioligand analogs and methods of using the compounds for the identification of 5-HT₃ receptors and the detection and treatment of abnormal conditions associated therewith.

U.S. Pat. No. 5,576,434 discloses a novel process for preparing 2-(1-azabicyclo[2.2.2]oct-3-yl)-2,3,3a,4,5,6-hexahydro-1H-benz[de]isoquinolin-1-one, the pharmaceutically acceptable salts thereof, which are 5-HT₃ receptor antagonists, and the intermediates thereof.

U.S. Pat. No. 5,561,149 discloses the use of a mono or bicyclic carbocyclic, or heterocyclic carboxylic, acid ester or amide or an imidazolyl carbazol in the manufacture of a medicament suitable for the treatment of stress-related psychiatric disorders, for increasing vigilance, for the treatment of rhinitis or serotonin-induced disorders and/or coadministration with another active agent to increase the bioavailability thereof, or for nasal administration.

U.S. Pat. No. 5,290,938 discloses optical active forms of the carboxylic acid amines of 3-aminoquinuclidine, generally N-(aminoquinuclidinyl-3)-alkylamides where alkyl is a linear or branched hydrocarbon chain of the general formula CₙH₍₂ₙ₊₁₎, preferably CH₃ or C₂H₅, and the preparation thereof. These can be hydrolyzed to the optical active forms of 3-aminoquinuclidine.

U.S. Pat. No. 5,273,972 discloses novel 2-substituted-3-quinuclidinyl arylcarboxamides and arylthiocarboxamides and corresponding arylcarboxylates which have utility as therapeutic agents which exhibit gastric prokinetic, antiemetic, anxiolytic and 5-HT (serotonin) antagonist effects in warm blooded animals.

U.S. Pat. No. 5,237,066 discloses enantiomers of absolute configuration S of amide derivatives of 3-aminoquinuclidine, the process for preparing them and their use as medicinal products having activity in respect of gastric movements and antiemetic activity.

U.S. Pat. No. 5,236,931 discloses novel 3-quinuclidinyl benzamides and benzoates which have utility as therapeutical agents which exhibit anxiolytic, antipsychotic, cognition improvement, antiemetic and gastric prokinetic effects in warm blooded animals.

U.S. Pat. No. 5,206,246 discloses anxiolytic-R—N-(1-azabicyclo[2.2.2]oct-3-yl)benzamides and thiobenzamides, their N-oxides and pharmaceutically acceptable salts thereof. A preferred compound is R-(+)-4-amino-N-(1-azabicyclo[2.2.2]oct-3-yl)-5-chloro-2-methoxybenzamide.

U.S. Pat. No. 5,106,843 discloses heterocyclic compounds useful as 5-HT₃ antagonists.

U.S. Pat. No. 5,084,460 discloses methods of therapeutic treatment with N-(3-quinuclidinyl)-2-hydroxybenzamides and thiobenzamides. The therapeutic agents are disclosed as exhibiting anxiolytic antipsychotic and cognitive improving effects in warm blooded animals.

U.S. Pat. No. 5,070,095 discloses novel 1-(azabicyclo[2.2.2]oct-3- or -4-yl)benzamides substituted on the benzene ring with the basic substituted aminomethyleneamino group which has been found to be useful in treating emesis, including emesis due to chemical and radiation anticancer therapy, anxiety, and impaired gastric emptying.

U.S. Pat. No. 5,057,519 discloses 5-HT₃ antagonists as being useful in reducing opiate tolerance.

U.S. Pat. No. 5,039,680 disclose 5-HT₃ antagonists in preventing or reducing dependency on dependency-inducing agents.

U.S. Pat. No. 5,025,022 discloses a method of treating or preventing schizophrenia and/or psychosis using S—N-(1-azabicyclo[2.2.2]oct-3-yl)benzamides and thiobenzamides, their N-oxides and pharmaceutically acceptable salts thereof. A preferred compound is S(−)-4-amino-N-(1-azabicyclo[2.2.2]oct-3-yl)-5-chloro-2-methoxybenzamide.

U.S. Pat. No. 5,017,580 discloses memory enhancing-R-N-(1-azabicyclo[2.2.2.]oct-3-yl)benzamides and thiobenzamides, their N-oxides and pharmaceutically acceptable salts thereof. A preferred compound is R-(+)-4-amino-N-(1-azabicyclo[2.2.2]oct-3-yl)-5-chloro-2-methoxybenzamide.

U.S. Pat. No. 4,908,370 discloses anxiolytic-N-(1-azabicyclo[2.2.2]oct-3-yl)benzamides and thiobenzamides as having anxiolytic activity, in particular, activity against anxiety induced by the withdrawal from ingested substances such as narcotics.

U.S. Pat. No. 4,877,794 discloses 2-alkoxy-N-(1-azabicyclo[2.2.2]oct-3-yl)benzamide and thiobenzamide compositions and the use thereof to treat schizophrenia.

U.S. Pat. No. 4,877,780 discloses antiemetic N-substituted benzamides having pharmaceutical properties rendering them useful as antiemetic agents with reduced undesirable side effects.

U.S. Pat. No. 4,870,181 discloses a process for the preparation of 2-alkoxy-N-(1-azabicyclo[2.2.2])octan-3-yl)aminobenzamide.

U.S. Pat. No. 4,835,162 discloses agonists and antagonists to nicotine as smoking deterrents.

U.S. Pat. No. 4,820,715 discloses anti-emetic quinuclidinyl benzamides. The compounds are particularly useful in the treatment of chemotherapy-induced emesis in cancer patients. Some of the compounds are also useful in disorders relating to impaired gastric motility.

U.S. Pat. No. 4,803,199 discloses pharmaceutically useful heterocyclic acid esters and amides or alkylene bridged peperidines as serotonin M antagonists.

U.S. Pat. No. 4,798,829 discloses 1-azabicyclo[3.2.2]nonane derivatives having gastric motility enhancing activity and/or anti-emetic activity and/or 5-HT receptor antagonist activity.

U.S. Pat. No. 4,721,720 discloses a method of treating emesis, anxiety and/or irritable bowel syndrome.

U.S. Pat. No. 4,717,563 discloses 2-alkoxy-N-(1-azabicyclo[2.2.2]oct-3-yl)benzamides and thiobenzamides in a method for alleviating emesis caused by non-platinum anticancer drugs.

U.S. Pat. No. 4,657,911 discloses 3-amino quinuclidine derivatives and the application thereof as accelerators of gastro-intestinal motor function and as medicament potentiators.

U.S. Pat. No. 4,605,652 discloses a method of enhancing memory or correcting memory deficiency with arylamido (and arylthioamido)-azabicycloalkanes, and the pharmaceutically acceptable acid addition salts, hydrates and alcoholates thereof.

U.S. Pat. No. 4,593,034 discloses 2-alkoxy-N-(1-azabicyclo[2.2.2]oct-3-yl)benzamides and thiobenzamides having gastrokinetic and anti-emetic activity.

U.S. Pat. No. 4,093,734 discloses amino-benzoic acid amides useful as anxiolytics, anticonvulsives, antiemetics and antiulcerogenics.

U.S. Pat. No. 3,702,324 discloses 3,4,5-trimethoxybenzamides of substituted anilines and of alkylpiperidines which exert a specific effect on the central nervous system and a somewhat lesser effect on muscle function, and thus have utility as tranquilizers.

WO 01/60821 discloses novel biarylcarboxamides.

WO 01/36417 A1 discloses novel N-azabicyclo-amide derivatives and use in therapy, especially in the treatment of prophylaxis of psychotic disorders and intellectual impairment disorders.

WO 01/29304 discloses quinuclidine acrylamides.

WO 00/73431 A2 discloses two binding assays to directly measure the affinity and selectivity of compounds at the α7 nAChR and the 5-HT₃R. The combined use of these functional and binding assays may be used to identify compounds that are selective agonists of the α7 nAChR.

WO 97/30998 discloses azabicyclic esters of carbamic acids useful in therapy.

WO 92/15579 discloses multicyclic tertiary amine polyaromatic squalene synthase inhibitors and method of treatment for lowering serum cholesterol levels using the compounds.

WO 92/11259 discloses azabicyclic amides or esters of halogenated benzoic acids having 5-HT₃ receptor antagonist activity.

WO 91/09593 discloses 5-HT₃ antagonists for treatment of nausea, bradycardia or hypotension associated myocardial instability.

FR 2 625 678 discloses N-(quinuclidin-3-yl)-benzamides and thiobenzamides useful as diet-control agents.

In Bioorg. & Med. Chem. Lett. 11 (2001) 319-321, the 5-HT₃ antagonist tropisetron (ICS 205-930) is discussed as a potent and selective α7 nicotinic receptor partial agonist.

In Behavioral Brain Res., 113 (2000) 169-181, it is discussed that the brain α7 nicotinic receptor may be an important therapeutic target for the treatment of Alzheimer's disease using DMXBA which is known as GTS-21.

In Eur. J.Med. Chem., 34 (1999) 415-422, benzimidazole-2-carboxylic acid amides and esters are discussed as a new structural class of 5-HT₃ ligands.

### SUMMARY OF THE INVENTION

In general, the invention includes a compound of formula A-L-B or a pharmaceutically acceptable salt thereof, wherein A is a 7-azabicyclo[2.2.1]heptane ring having 1S, 2R, and 4R stereochemistry; L is a linking moiety including an amide, a thioamide, an acrylamide, an acrylthioamide, a propiolamide, or a propiolthioamide where the linking moiety is bonded to the C-2 carbon of the heptane ring in an exo orientation; and B is phenyl, naphthyl, or phenyl fused to a 5- or 6-membered saturated or partially unsaturated ring, all optionally substituted where valency allows with any one or more of the following substituents as herein defined: alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —NO₂, —CN, F, Cl, Br, I, —OR₈, —SR₈, —S(O)₂R₈, —S(O)R₈, —OS(O)₂R₈, —N(R₈)₂, —C(O)R₈, —C(S)R₈, —C(O))R₈, —C(O)N(R₈)₂, —NR₈C(O)R₈, —S(O)₂N(R₈)₂, —NR₈S(O)₂R₈, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally a carbon atom is substituted with ═O or ═S where valency allows. The B is bonded to L wherever valency allows on B.

The present invention discloses compounds of the Formula I:

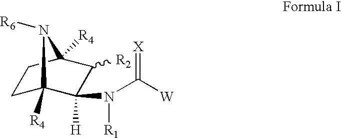

wherein the stereochemistry of the of the 7-azabicyclo[2.2.1]heptane ring is 1S, 4R and the nitrogen substituent at the C-2 carbon has the exo orientation and is R;

X is O or S;

W is -Q, —C═C-Q, or —C≡C-Q;

Q is aryl wherein the aryl can have a bond to the core molecule at any position where valency allows provided that there is only one said bond to the core molecule, or a group of formula II

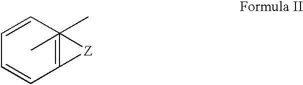

wherein the phenyl ring of formula II is optionally substituted with 1-4 substituents independently selected from F, Cl, Br, I, R₃, or a bond to the core molecule at any position where valency allows, provided that there is only one said bond to the core molecule;

Z is —C(R_{Z})₂—C(R_{Z})₂—C(R_{Z})₂—, —C(R_{Z})═C(R_{Z})—C(R_{Z})₂—, —C(R_{Z})₂—C(R_{Z})₂—C(R_{Z})₂—C(R_{Z})₂—, —C(R_{Z})═C(R_{Z})—C(R_{Z})₂—C(R_{Z})₂—, or —C(R_{Z})₂—C(R_{Z})═C(R_{Z})—C(R_{Z})₂—;

R_{Z} is H, R₃, or a bond to the core molecule at any position where valency allows, provided that there is only one said bond to the core molecule;

R₁ is H, alkyl, cycloalkyl, halogenated alkyl, or aryl;

R₂ is H, alkyl, halogenated alkyl, substituted alkyl, cycloalkyl, or aryl;

Each R₃ is independently alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —OR₈, —SR₈, —S(O)₂R₈, —S(O)R₈, —OS(O)₂R₈, F, Cl, Br, I, —N(R₈)₂, —C(O)R₈, —C(S)R₈, —C(O)OR₈, —CN, —C(O)N(R₈)₂, —NR₈C(O)R₈, —S(O)₂N(R₈)₂, —NR₈S(O)₂R₈, —NO₂, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally two R₃ groups bound to the same carbon atom together form ═O or ═S;

Each R₄ is independently H, alkyl, or substituted alkyl;

R₆ is H, alkyl, an amino protecting group, or an alkyl group having 1-3 substituents selected from F, Cl, Br, I, —OH, —CN, —NH₂, —NH(alkyl), or —N(alkyl)₂;

R₇ is 5-membered heteroaromatic mono-cyclic moieties containing within the ring 1-3 heteroatoms independently selected from the group consisting of ═N—, —N(R₂₀)—, —O—, and —S—, and having 0-1 substituent selected from R₁₇ and further having 0-3 substituents independently selected from F, Cl, Br, or I, or R₇ is 9-membered fused-ring moieties having a 6-membered ring fused to a 5-membered ring including the formula

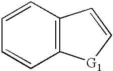

wherein G₁ is O, S or NR₂₀,

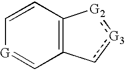

wherein G is C(R₁₄) or N, and each G₂ and G₃ are independently selected from C(R₁₄)₂, C(R₁₄), O, S, N, and N(R₂₀), provided that both G₂ and G₃ are not simultaneously O or S, or

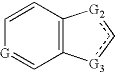

wherein G is C(R₁₄) or N, and each G₂ and G₃ are independently selected from C(R₁₄)₂, C(R₁₄), O, S, N, and N(R₂₀), each 9-membered bicyclic ring having 0-1 substituent selected from R₁₇ and 0-3 substituents independently selected from F, Cl, Br, or I, wherein the R₇ moiety attaches to other substituents as defined in formula I at any position on either ring as valency allows;

Each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅;

R₉ is 6-membered heteroaromatic mono-cyclic moieties containing within the ring 1-3 heteroatoms selected from ═N— and having 0-1 substituent selected from R₁₇ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₇ and 0-3 substituent(s) independently selected from F, Cl, Br, or I, wherein the R₉ moiety attaches to other substituents as defined in formula I at any position on either ring as valency allows;

Each R₁₀ is independently H, alkyl, cycloalkyl, heterocycloalkyl, R₇, R₉, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, or phenyl optionally substituted with 1-4 substituents independently selected from F, Cl, Br, I, R₁₃, and R₁₅;

Each R₁₁ is independently H, alkyl, cycloalkyl, heterocycloalkyl, halogenated alkyl, halogenated cycloalkyl, or halogenated heterocycloalkyl;

R₁₂ is alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —S(O)R₁₁, —S(O)₂R₁₁, —OS(O)₂R₁₁, —NR₁₁R₁₁, —C(O)R₁₁, —C(S)R₁₁, —C(O)OR₁₁, —NO₂, —CN, —C(O)N(R₁₁)₂, —NR₁₁C(O)R₁₁, —NR₁₁C(O)N(R₁₁)₂, —S(O)₂N(R₁₁)₂, or —NR₁₁S(O)₂R₁₁;

R₁₃ is —OR₁₁, —SR₁₁, —SOR₁₁, —SO₂R₁₁, —OSO₂R₁₁, —N(R₁₁)₂, —C(O)R₁₁, —C(O)OR₁₁, —C(S)R₁₁, —C(O)N(R₁₁)₂, —NO₂—CN, —CF₃, —NR₁₁C(O)R₁₁, —NR₁₁C(O)N(R₁₁)₂, —S(O)₂N(R₁₁)₂, or —NR₁₁S(O)₂R₁₁;

R₁₄ is H or R₁₉;

R₁₅ is lactam heterocycloalkyl, R₇, R₉, or R₁₉;

Each R₁₆ is independently H, alkyl, cycloalkyl, halogenated alkyl, or halogenated cycloalkyl;

R₁₇ is alkyl, cycloalkyl, or heterocycloalkyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, and —NR₁₆S(O)₂R₁₆, and the cycloalkyl and heterocycloalkyl also being further optionally substituted with ═O or ═S;

R₁₉ is alkyl, cycloalkyl, heterocycloalkyl, phenyl, or naphthyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, or —NR₁₆S(O)₂R₁₆, and the cycloalkyl and heterocycloalkyl also being further optionally substituted with ═O or ═S;

R₂₀ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₂ and further having 0-3 substituents independently selected from F, Cl, Br, or I;

or pharmaceutical composition, pharmaceutically acceptable salt, racemic mixture, or pure enantiomer thereof.

Another embodiment of the present invention provides a use of a compound of Formula I or formula A-L-B for the preparation of a medicament for treating a disease or condition, wherein the diseases, disorders, and/or condition is any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

Another embodiment of the present invention provides a method of treating or preventing diseases, disorders, and/or conditions using a compound of Formula I or formula A-L-B wherein the diseases, disorders, and/or condition is any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

In another aspect, the invention includes treating a mammal suffering from schizophrenia or psychosis by administering compounds of formula A-L-B or Formula I in conjunction with antipsychotic drugs (also called anti-psychotic agents). The compounds of the present invention and the antipsychotic drugs can be administered simultaneously or at separate intervals. When administered simultaneously the compounds of the present invention and the antipsychotic drugs can be incorporated into a single pharmaceutical composition. Alternatively, two separate compositions, i.e., one containing compounds of the present invention and the other containing antipsychotic drugs, can be administered simultaneously.

The present invention also includes the compounds of the present invention, pharmaceutical compositions containing the active compounds as the free base or as a pharmaceutically acceptable salt and a pharmaceutically acceptable carrier, and methods to treat the identified diseases.

A further embodiment of the present invention provides a method comprising administering a therapeutically effective amount of a compound of the present invention or a pharmaceutical composition contains said compound to the mammal.

Embodiments of the invention may include one or more or combination of the following.

The compound of Formula I, wherein X is O.

The compound of Formula I, where X is S.

The compound of Formula I, where R₁ is H, alkyl, or cycloalkyl, and where R₂ is H, alkyl, substituted alkyl, cycloalkyl, halogenated alkyl, or aryl.

The compound of Formula I, where Q is aryl.

The compound of Formula I, where Q is formula II.

The compound of Formula I, where formula II includes indanyl, indenyl, dihydronaphthyl, or tetrahydronaphthyl.

The compound of Formula I, where aryl is any one or more or combination of the following: phenyl, substituted phenyl, naphthyl, or substituted naphthyl.

The compound of Formula I, where each R₄ is independently H, lower alkyl, or substituted lower alkyl.

The compound of Formula I, where R₆ is an amino protecting group.

The compound of Formula I, where R₆ is H, or lower alkyl optionally substituted with up to 3 substituents independently selected from F, Cl, Br, I, —OH, —CN, —NH₂, —NH(alkyl), or —N(alkyl)₂.

The compound of Formula I, where R₁ is H or lower alkyl, and where R₂ is H or lower alkyl.

The compound of Formula I, where at least one R₄ is H and one R₄ is H or lower alkyl optionally substituted with 1 substituent selected from —CN, —NO₂, —OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀, or phenyl optionally substituted with up to 4 substitutents independently selected from F, Cl, Br, I, R₁₃, and R₁₅, provided that when said lower alkyl is optionally substituted, said lower alkyl can be further optionally substituted with up to 3 substituents independently selected from F, Cl, Br, and I, and further provided that R₁₀ is H, lower alkyl, or halogenated lower alkyl. This allows the lower alkyl of R₄ to be substituted with one substituent selected from —CN, —NO₂, —OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀, or phenyl optionally substituted with up to 4 substitutents independently selected from F, Cl, Br, I, R₁₃, and R₁₅, and further optionally substituted with up to 3 substituents independently selected from F, Cl, Br, and I on any carbon with sufficient valency for said substitution. This further provides that R₁₀ is H, lower alkyl or halogenated lower alkyl for the following optional substituents on R_{4:—}OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀.

The compound of Formula I, where R₁, R₂, and each R₄ are H.

The compound of Formula I or formula A-L-B, where the compound is any one or more or combination of the following as the free base, or a pharmaceutically acceptable salt thereof: 3-amino-N-[(1S,2R,4R)-7-azabicyclo[2.2.1]hept-2-yl]-4-hydroxybenzamide; or N-[(1S,2R,4R)-7-azabicyclo[2.2.1]hept-2-yl]-4-(4-fluorophenoxy)benzamide.

The compound of Formula I or formula A-L-B, where the compound is any one or more or combination of the following as the free base, or a pharmaceutically acceptable salt thereof: N-[(1S,2R,4R)-7-azabicyclo[2.2.1]hept-2-yl]-4-(4-hydroxyphenoxy)benzamide; N-[(1S,2R,4R)-7-azabicyclo[2.2.1]hept-2-yl]-4-(4-acetamidophenoxy)benzamide; N-[(1S,2R,4R)-7-azabicyclo[2.2.1]hept-2-yl]-4-phenoxybenzamide; N-[(1S,2R,4R)-7-azabicyclo[2.2.1]hept-2-yl]-4-benzylbenzamide; N-[(1S,2R,4R)-7-azabicyclo[2.2.1]hept-2-yl]-4-(phenylsulfanyl)benzamide; N-[(1S,2R,4R)-7-azabicyclo[2.2.1]hept-2-yl]-3-phenoxybenzamide; N-[(1S,2R,4R)-7-azabicyclo[2.2.1]hept-2-yl]-4-benzoylbenzamide; N-[(1S,2R,4R)-7-azabicyclo[2.2.1]hept-2-yl]-4-(2-fluorophenoxy)benzamide; N-[(1S,2R,4R)-7-azabicyclo[2.2.1]hept-2-yl]-4-(3-fluorophenoxy)benzamide; N-[(1S,2R,4R)-7-azabicyclo[2.2.1]hept-2-yl]-4-(2-chlorophenoxy)benzamide; N-[(1S,2R,4R)-7-azabicyclo[2.2.1]hept-2-yl]-4-(3-chlorophenoxy)benzamide; N-[(1S,2R,4R)-7-azabicyclo[2.2.1]hept-2-yl]-4-(4-chlorophenoxy)benzamide; N-[(1S,2R,4R)-7-azabicyclo[2.2.1]hept-2-yl]-4-(2-methoxyphenoxy)benzamide; N-[(1S,2R,4R)-7-azabicyclo[2.2.1]hept-2-yl]-4-(3-methoxyphenoxy)benzamide; N-[(1S,2R,4R)-7-azabicyclo[2.2.1]hept-2-yl]-4-(4-methoxyphenoxy)benzamide; N-[(1S,2R,4R)-7-azabicyclo[2.2.1]hept-2-yl]-4-(3-chlorophenylsulfanyl)benzamide; N-[(1S,2R,4R)-7-azabicyclo[2.2.1]hept-2-yl]-4-(4-chlorophenylsulfanyl)benzamide; N-[(1S,2R,4R)-7-azabicyclo[2.2.1]hept-2-yl]-4-(2-chlorophenylsulfanyl)benzamide; N-[(1S,2R,4R)-7-azabicyclo[2.2.1]hept-2-yl]-4-(4-methoxyphenylsulfanyl)-benzamide; N-[(1S,2R,4R)-7-azabicyclo[2.2.1]hept-2-yl]-4-(3-methoxyphenylsulfanyl)-benzamide; N-[(1S,2R,4R)-7-azabicyclo[2.2.1]hept-2-yl]-4-(2-methoxyphenylsulfanyl)-benzamide; N-[(1S,2R,4R)-7-azabicyclo[2.2.1]hept-2-yl]-4-phenoxybenzamide; N-[(1S,2R,4R)-7-azabicyclo[2.2.1]hept-2-yl]-4-(4-aminophenoxy)-benzamide; N-[(1S,2R,4R)-7-azabicyclo[2.2.1]hept-2-yl]-4-(3-aminophenoxy)-benzamide; N-[(1S,2R,4R)-7-azabicyclo[2.2.1]hept-2-yl]-4-(2-aminophenoxy)-benzamide; N-[(1S,2R,4R)-7-azabicyclo[2.2.1]hept-2-yl]-4-(4-methanesulfonylamino-phenoxy)-benzamide; N-[(1S,2R,4R)-7-azabicyclo[2.2.1]hept-2-yl]-4-(3-methanesulfonylamino-phenoxy)-benzamide; N-[(1S,2R,4R)-7-azabicyclo[2.2.1]hept-2-yl]-4-(2-methanesulfonylamino-phenoxy)-benzamide; N-[(1S,2R,4R)-7-azabicyclo[2.2.1]hept-2-yl]-4-(4-acetoxyphenoxy)-benzamide; N-[(1S,2R,4R)-7-azabicyclo[2.2.1]hept-2-yl]-4-(3-acetoxyphenoxy)-benzamide; N-[(1S,2R,4R)-7-azabicyclo[2.2.1]hept-2-yl]-4-(2-acetoxyphenoxy)-benzamide; N-[(1S,2R,4R)-7-azabicyclo[2.2.1]hept-2-yl]-4-(4-acetylphenoxy)-benzamide; N-[(1S,2R,4R)-7-azabicyclo[2.2.1]hept-2-yl]-4-(3-acetylphenoxy)-benzamide; N-[(1S,2R,4R)-7-azabicyclo[2.2.1]hept-2-yl]-4-(2-acetylphenoxy)-benzamide; N-[(1S,2R,4R)-7-azabicyclo[2.2.1]hept-2-yl]-4-(4-carbamoylphenoxy)-benzamide; N-[(1S,2R,4R)-7-azabicyclo[2.2.1]hept-2-yl]-4-(3-carbamoylphenoxy)-benzamide; N-[(1S,2R,4R)-7-azabicyclo[2.2.1]hept-2-yl]-4-(2-carbamoylphenoxy)-benzamide; N-[(1S,2R,4R)-7-azabicyclo[2.2.1]hept-2-yl]-4-(4-cyanophenoxy)-benzamide; N-[(1S,2R,4R)-7-azabicyclo[2.2.1]hept-2-yl]-4-(3-cyanophenoxy)-benzamide; N-[(1S,2R,4R)-7-azabicyclo[2.2.1]hept-2-yl]-4-(2-cyanophenoxy)-benzamide; N-[(1S,2R,4R)-7-azabicyclo[2.2.1]hept-2-yl]-4-(4-sulfamoylphenoxy)-benzamide; N-[(1S,2R,4R)-7-azabicyclo[2.2.1]hept-2-yl]-4-(3-sulfamoylphenoxy)-benzamide; N-[(1S,2R,4R)-7-azabicyclo[2.2.1]hept-2-yl]-4-(2-sulfamoylphenoxy)-benzamide; N-[(1S,2R,4R)-7-azabicyclo[2.2.1]hept-2-yl]-4-(thiophen-2-yloxy)-benzamide; N-[(1S,2R,4R)-7-azabicyclo[2.2.1]hept-2-yl]-4-(5-methylthiophen-2-yloxy)-benzamide; N-[(1S,2R,4R)-7-azabicyclo[2.2.1]hept-2-yl]-4-(5-chlorothiophen-2-yloxy)-benzamide; N-[(1S,2R,4R)-7-azabicyclo[2.2.1]hept-2-yl]-4-(5-methoxythiophen-2-yloxy)-benzamide; N-[(1S,2R,4R)-7-azabicyclo[2.2.1]hept-2-yl]-4-(5-trifluoromethylthiophen-2-yloxy)-benzamide; N-[(1S,2R,4R)-7-azabicyclo[2.2.1]hept-2-yl]-4-(5-acetylthiophen-2-yloxy)-benzamide; N-λ(1S, 2R, 4R)-7-azabicyclo[2.2.1]hept-2-yl]-4(5-acetaminothiophen-2-yloxy)-benzamide; N-[(1S,2R,4R)-7-azabicyclo[2.2.1]hept-2-yl]-4-(5-cyanothiophen-2-yloxy)-benzamide; N-[(1S,2R,4R)-7-azabicyclo[2.2.1]hept-2-yl]-4-(5-morpholin-4-yl-thiophen-2-yloxy)-benzamide; N-[(1S,2R,4R)-7-azabicyclo[2.2.1]hept-2-yl]-4-(4-methylthiophen-2-yloxy)-benzamide; N-[(1S,2R,4R)-7-azabicyclo[2.2.1]hept-2-yl]-4-(4-chlorothiophen-2-yloxy)-benzamide; N-[(1S,2R,4R)-7-azabicyclo[2.2.1]hept-2-yl]-4-(4-methoxythiophen -2-yloxy)-benzamide; N-[(1S,2R,4R)-7-azabicyclo[2.2.1]hept-2-yl]-4-(4-trifluoromethylthiophen-2-yloxy)-benzamide; N-[(1S,2R,4R)-7-azabicyclo[2.2.1]hept-2-yl]-4-(4-acetylthiophen-2-yloxy)-benzamide; N-[(1S,2R, 4R)-7-azabicyclo[2.2.1]hept-2-yl]-4-(4-acetaminothiophen-2-yloxy)-benzamide; N-[(1S,2R,4R)-7-azabicyclo[2.2.1]hept-2-yl]-4-(4-cyanothiophen-2-yloxy)-benzamide; N-[(1S,2R,4R)-7-azabicyclo[2.2.1]hept-2-yl]-4-(4-morpholin-4-yl-thiophen -2-yloxy)-benzamide; N-[(1S,2R,4R)-7-azabicyclo[2.2.1]hept-2-yl]-4-(furan -2-yloxy)-benzamide; N-[(1S,2R,4R)-7-azabicyclo[2.2.1]hept-2-yl]-4-(5-methylfuran -2-yloxy)-benzamide; N-[(1S,2R,4R)-7-azabicyclo[2.2.1]hept-2-yl]-4-(5-chlorofuran -2-yloxy)-benzamide; N-[(1S,2R,4R)-7-azabicyclo[2.2.1]hept-2-yl]-4-(5-methoxyfuran -2-yloxy)-benzamide; N-[(1S,2R,4R)-7-azabicyclo[2.2.1]hept-2-yl]-4-(5-trifluoromethylfuran-2-yloxy)-benzamide; N-[(1S,2R,4R)-7-azabicyclo[2.2.1]hept-2-yl]-4-(5-acetylfuran-2-yloxy)-benzamide; N-[(1S,2R,4R) -7-azabicyclo[2.2.1]hept-2-yl]-4-(5-acetaminofuran-2-yloxy)-benzamide; N-[(8S,2R,4R)-7-azabicyclo[2.2.1]hept-2-yl]-4-(5-cyanofuran-2-yloxy)-benzamide; N-[(1S,2R4R)-7-azabicyclo[2.2.1]hept-2-yl]-4-(5-morpholin-4-yl-furan-2-yloxy)-benzamide; N-[(1S,2R,4R)-7-azabicyclo[2.2.1]hept-2-yl]-4-(4-methylfuran-2-yloxy)-benzamide; N-[(1S,2R,4R)-7-azabicyclo[2.2.1]hept-2-yl]-4-(4-chlorofuran -2-yloxy)-benzamide; N-[(1S,2R,4R)-7-azabicyclo[2.2.1]hept-2-yl]-4-(4-methoxyfuran -2-yloxy)-benzamide; N-[(1S,2R,4R)-7-azabicyclo[2.2.1]hept-2-yl]-4-(4-trifluoromethylfuran -2-yloxy)-benzamide; N-[(1S,2R,4R)-7-azabicyclo[2.2.1]hept-2-yl]-4-(4-acetylfuran -2-yloxy)-benzamide; N-[(1S,2R,4R)-7-azabicyclo[2.2.1]hept-2-yl]-4-(4-acetaminofuran -2-yloxy)-benzamide; N-[(1S,2R,4R)-7-azabicyclo[2.2.1]hept-2-yl]-4-(4-cyanofuran-2-yloxy)-benzamide; N-[(1S,2R,4R)-7-azabicyclo[2.2.1]hept-2-yl]-4-(4-morpholin-4-yl-furan-2-yloxy)-benzamide; N-[(1S,2R,4R)-7-azabicyclo[2.2.1]hept-2-yl]-4-(oxazol-2-yloxy)-benzamide; N-[(1S,2R,4R)-7-azabicyclo[2.2.1]hept-2-yl]-4-(5-methyloxazol-2-yloxy)-benzamide; N-[(1S,2R,4R) -7-azabicyclo[2.2.1]hept-2-yl]-4-(5-chlorooxazol-2-yloxy)-benzamide; N-[(1S,2R,4R)-7-azabicyclo[2.2.1]hept-2-yl]-4-(5-methoxyoxazol-2-yloxy)-benzamide; N-[(1S,2R,4R)-7-azabicyclo[2.2.1]hept-2-yl]-4-(5-trifluoromethyloxazol-2-yloxy)-benzamide; N-[(1S,2R,4R)-7-azabicyclo[2.2.1]hept-2-yl]-4-(5-acetyloxazol -2-yloxy)-benzamide; N-[(1S,2R,4R)-7-azabicyclo[2.2.1]hept-2-yl]-4-(5-acetaminooxazol -2-yloxy)-benzamide; N-[(1S,2R,4R)-7-azabicyclo[2.2.1]hept-2-yl]-4-(5-cyanooxazol-2-yloxy)-benzamide; N-[(1S,2R,4R)-7-azabicyclo[2.2.1]hept-2-yl]-4-(5-morpholin-4-yl-oxazol-2-yloxy)-benzamide; N-[(1S,2R,4R)-7-azabicyclo[2.2.1]hept-2-yl]-4-(oxazol-2-yloxy)-benzamide; N-[(1S,2R,4R)-7-azabicyclo[2.2.1]hept-2-yl]-4-(4-methyloxazol-2-yloxy)-benzamide; N-[(1S,2R,4R) -7-azabicyclo[2.2.1]hept-2-yl]-4-(4-chlorooxazol-2-yloxy)-benzamide; N-[(1S,2R, 4R)-7-azabicyclo[2.2.1]hept-2-yl]-4-(4-methoxyoxazol-2-yloxy)-benzamide; N-[(1S,2R,4R)-7-azabicyclo[2.2.1]hept-2-yl]-4-(4-trifluoromethyloxazol-2-yloxy)-benzamide; N-[(1S,2R,4R)-7-azabicyclo[2.2.1]hept-2-yl]-4-(4-acetyloxazol -2-yloxy)-benzamide; N-[(1S,2R,4R)-7-azabicyclo[2.2.1]hept-2-yl]-4-(4-acetaminooxazol-2-yloxy)-benzamide; N-[(1S,2R,4R)-7-azabicyclo[2.2.1]hept-2-yl]-4-(4-cyanooxazol-2-yloxy)-benzamide; N-[(1S,2R,4R)-7-azabicyclo[2.2.1]hept-2-yl]-4-(4-morpholin-4-yl-oxazol-2-yloxy)-benzamide; N-[(1S,2R,4R)-7-azabicyclo[2.2.1]hept-2-yl]-4-(oxazol-5-yloxy)-benzamide; N-[(1S,2R,4R)-7-azabicyclo[2.2.1]hept-2-yl]-4-(2-methyloxazol-5-yloxy)-benzamide; N-[(1S,2R,4R) -7-azabicyclo[2.2.1]hept-2-yl]-4-(2-chlorooxazol-5-yloxy)-benzamide; N-[(1S,2R,4R)-7-azabicyclo[2.2.1]hept-2-yl]-4-(2-methoxyoxazol-5-yloxy)-benzamide; N-[(1S,2R,4R)-7-azabicyclo[2.2.1]hept-2-yl]-4-(2-trifluoromethyloxazol-5-yloxy)-benzamide; N-[(1S,2R,4R)-7-azabicyclo[2.2.1]hept-2-yl]-4-(2-acetyloxazol -5-yloxy)-benzamide; N-[(1S,2R,4R)-7-azabicyclo[2.2.1]hept-2-yl]-4-(2-acetaminooxazol -5-yloxy)-benzamide; N-[(1S,2R,4R)-7-azabicyclo[2.2.1]hept-2-yl]-4-(2-cyanooxazol-5-yloxy)-benzamide; N-[(1S,2R,4R)-7-azabicyclo[2.2.1]hept-2-yl]-4-(2-morpholin-4-yl-oxazol-5-yloxy)-benzamide; N-[(1S,2R,4R)-7-azabicyclo[2.2.1]hept-2-yl]-4-(thiazol-2-yloxy)-benzamide; N-[(1S,2R,4R)-7-azabicyclo[2.2.1]hept-2-yl]-4-(5-methylthiazol-2-yloxy)-benzamide; N-[(1S,2R,4R)-7-azabicyclo[2.2.1]hept-2-yl]-4-(5-chlorothiazol-2-yloxy)-benzamide; N-[(1S,2R,4R)-7-azabicyclo[2.2.1]hept-2-yl]-4-(5-methoxythiazol-2-yloxy)-benzamide; N-[(1S,2R,4R)-7-azabicyclo[2.2.1]hept-2-yl]-4-(5-trifluoromethylthiazol -2-yloxy)-benzamide; N-[(1S,2R,4R)-7-azabicyclo[2.2.1]hept-2-yl]-4-(5-acetylthiazol -2-yloxy)-benzamide; N-[(1S,2R,4R)-7-azabicyclo[2.2.1]hept-2-yl]-4-(5-acetaminothiazol -2-yloxy)-benzamide; N-[(1S,2R,4R)-7-azabicyclo[2.2.1]hept-2-yl]-4-(5-cyanothiazol-2-yloxy)-benzamide; N-[(1S,2R,4R)-7-azabicyclo[2.2.1]hept-2-yl]-4-(5-morpholin-4-yl-thiazol-2-yloxy)-benzamide; N-[(1S,2R,4R)-7-azabicyclo[2.2.1]hept-2-yl]-4-(thiazol-2-yloxy)-benzamide; N-[(1S,2R,4R)-7-azabicyclo-[2.2.1]hept-2-yl]-4-(4-methylthiazol-2-yloxy)-benzamide; N-[(1S,2R,4R)-7-azabicyclo[2.2.1]hept-2-yl]-4-(4-chlorothiazol-2-yloxy)-benzamide; N-[(1S,2R,4R)-7-azabicyclo[2.2.1]hept-2-yl]-4-(4-methoxythiazol-2-yloxy)-benzamide; N-[(1S,2R,4R)-7-azabicyclo[2.2.1]hept-2-yl]-4-(4-trifluoromethylthiazol -2-yloxy)-benzamide; N-[(1S,2R,4R)-7-azabicyclo[2.2.1]hept-2-yl]-4-(4-acetylthiazol -2-yloxy)-benzamide; N-[(1S,2R,4R)-7-azabicyclo[2.2.1]hept-2-yl]-4-(4-acetaminothiazol -2-yloxy)-benzamide; N-[(1S,2R,4R)-7-azabicyclo[2.2.1]hept-2-yl]-4-(4-cyanothiazol-2-yloxy)-benzamide; N-[(1S,2R,4R)-7-azabicyclo[2.2.1]hept-2-yl]-4-(4-morpholin-4-yl-thiazol-2-yloxy)-benzamide; N-[(1S,2R,4R)-7-azabicyclo[2.2.1]hept-2-yl]-4-(thiazol-5-yloxy)-benzamide; N-[(1S,2R,4R)-7-azabicyclo[2.2.1]hept-2-yl]-4-(2-methylthiazol-5-yloxy)-benzamide; N-[(1S,2R,4R)-7-azabicyclo[2.2.1]hept-2-yl]-4-(2-chlorothiazol-5-yloxy)-benzamide; N-[(1S,2R,4R)-7-azabicyclo[2.2.1]hept-2-yl]-4-(2-methoxythiazol-5-yloxy)-benzamide; N-[(1S,2R,4R)-7-azabicyclo[2.2.1]hept-2-yl]-4-(2-trifluoromethylthiazol-5-yloxy)-benzamide; N-[(1S,2R,4R)-7-azabicyclo[2.2.1]hept-2-yl]-4-(2-acetylthiazol -5-yloxy)-benzamide; N-[(1S,2R,4R)-7-azabicyclo[2.2.1]hept-2-yl]-4-(2-acetaminothiazol -5-yloxy)-benzamide; N-[(1S,2R,4R)-7-azabicyclo[2.2.1]hept-2-yl]-4-(2-cyanothiazol-5-yloxy)-benzamide; N-[(1S,2R,4R)-7-azabicyclo[2.2.1]hept-2-yl]-4-(2-morpholin-4-yl-thiazol-5-yloxy)-benzamide; N-[(1S,2R,4R)-7-azabicyclo[2.2.1]hept-2-yl]-4-([1,3,4]oxadiazol-2-yloxy)-benzamide; N-[(1S,2R,4R)-7-azabicyclo[2.2.1]hept-2-yl]-4-(5-methyl[1,3,4]oxadiazol-2-yloxy)-benzamide; N-[(1S,2R,4R)-7-azabicyclo[2.2.1]hept-2-yl]-4-(5-chloro[1,3,4]oxadiazol -2-yloxy)-benzamide; N-[(1S,2R,4R)-7-azabicyclo[2.2.1]hept-2-yl]-4-(5-methoxy[1,3,4]oxadiazol-2-yloxy)-benzamide; N-[(1S,2R,4R)-7-azabicyclo[2.2.1]hept-2-yl]-4-(5-trifluoromethyl[1,3,4]oxadiazol-2-yloxy)-benzamide; N-[(1S,2R,4R)-7-azabicyclo[2.2.1]hept-2-yl]-4-(5-acetyl[1,3,4]oxadiazol -2-yloxy)-benzamide; N-[(1S,2R,4R)-7-azabicyclo[2.2.1]hept-2-yl]-4-(5-acetamino[1,3,4]oxadiazol-2-yloxy)-benzamide; N-[(1S,2R,4R)-7-azabicyclo[2.2.1]hept-2-yl]-4-(5-cyano[1,3,4]oxadiazol-2-yloxy)-benzamide; N-[(1S,2R,4R)-7-azabicyclo[2.2.1]hept-2-yl]-4-(5-morpholin-4-yl-[1,3,4]oxadiazol-2-yloxy)-benzamide; N-[(1S,2R,4R)-7-azabicyclo[2.2.1]hept-2-yl]-4-([1,3,4]thiadiazol -2-yloxy)-benzamide; N-[(1S,2R,4R)-7-azabicyclo[2.2.1]hept-2-yl]-4-(5-methyl[1,3,4]thiadiazol-2-yloxy)-benzamide; N-[(1S,2R,4R)-7-azabicylo[2.2.1]hept-2-yl]-4-(5-chloro[1,3,4]thiadiazol-2-yloxy)-benzamide; N-[(1S,2R,4R)-7-azabicyclo[2.2.1]hept-2-yl]-4-(5-methoxy[1,3,4]thiadiazol-2-yloxy)-benzamide; N-[(1S,2R,4R)-7-azabicyclo[2.2.1]hept-2-yl]-4-(5-trifluoromethyl[1,3,4]thiadiazol-2-yloxy)-benzamide; N-[(1S,2R,4R)-7-azabicyclo[2.2.1]hept-2-yl]-4-(5-acetyl[1,3,4]thiadiazol-2-yloxy)-benzamide; N-[(1S,2R,4R)-7-azabicyclo[2.2.1]hept-2-yl]-4-(5-acetamino[1,3,4]thiadiazol -2-benzamide; N-[(1S,2R,4R)-7-azabicyclo[2.2.1]hept-2-yl]-4-(5-cyano[1,3,4]thiadiazol-2-yloxy)-benzamide; N-[(1S,2R,4R)-7-azabicyclo[2.2.1]hept-2-yl]-4-(5-morpholin-4-yl-[1,3,4]thiadiazol-2-yloxy)-benzamide; N-[(1S,2R,4R)-7-azabicyclo[2.2.1]hept-2-yl]-4-(4-aminophenylsulfanyl)-benzamide; N-[(1S,2R,4R)-7-azabicyclo[2.2.1]hept-2-yl]-4-(3-aminophenylsulfanyl)-benzamide; N-[(1S,2R,4R)-7-azabicyclo[2.2.1]hept-2-yl]-4-(2-aminophenylsulfanyl)-benzamide; N-[(1S,2R,4R)-7-azabicyclo[2.2.1]hept-2-yl]-4-(4-methanesulfonylamino-phenylsulfanyl)-benzamide; N-[(1S,2R,4R)-7-azabicyclo[2.2.1]hept-2-yl]-4-(3-methanesulfonylamino-phenylsulfanyl)-benzamide; N-[(1S,2R,4R)-7-azabicyclo[2.2.1]hept-2-yl]-4-(2-methanesulfonylamino-phenylsulfanyl)-benzamide; N-[(1S,2R,4R)-7-azabicyclo[2.2.1]hept-2-yl]-4-(4-acetoxyphenylsulfanyl)-benzamide; N-[(1S,2R,4R)-7-azabicyclo[2.2.1]hept-2-yl]-4-(3-acetoxyphenylsulfanyl)-benzamide; N-[(1S,2R,4R)-7-azabicyclo[2.2.1]hept-2-yl]-4-(2-acetoxyphenylsulfanyl)-benzamide; N-[(1S,2R,4R)-7-azabicyclo[2.2.1]hept-2-yl]-4-(4-acetylphenylsulfanyl)-benzamide; N-[(1S,2R,4R) -7-azabicyclo[2.2.1]hept-2-yl]-4-(3-acetylphenylsulfanyl)-benzamide; N-[(1S,2R,4R)-7-azabicyclo[2.2.1]hept-2-yl]-4-(2-acetylphenylsulfanyl)-benzamide; N-[(1S,2R,4R)-7-azabicyclo[2.2.1]hept-2-yl]-4-(4-carbamoylphenylsulfanyl)-benzamide; N-[(1S,2R,4R)-7-azabicyclo[2.2.1]hept-2-yl]-4-(3-carbamoylphenylsulfanyl)-benzamide; N-[(1S,2R,4R)-7-azabicyclo[2.2.1]hept-2-yl]-4-(2-carbamoylphenylsulfanyl)-benzamide; N-[(1S,2R,4R)-7-azabicyclo[2.2.1]hept-2-yl]-4-(4-cyanophenylsulfanyl)-benzamide; N-[(1S,2R,4R)-7-azabicyclo[2.2.1]hept-2-yl]-4-(3-cyanophenylsulfanyl)-benzamide; N-[(1S,2R,4R)-7-azabicyclo[2.2.1]hept-2-yl]-4-(2-cyanophenylsulfanyl)-benzamide; N-[(1S,2R,4R) -7-azabicyclo[2.2.1]hept-2-yl]-4-(4-sulfamoylphenylsulfanyl)-benzamide; N-[(1S,2R,4R)-7-azabicyclo[2.2.1]hept-2-yl]-4-(3-sulfamoylphenylsulfanyl)-benzamide; N-[(1S,2R,4R)-7-azabicyclo[2.2.1]hept-2-yl]-4-(2-sulfamoylphenylsulfanyl)-benzamide; N-[(1S,2R,4R)-7-azabicyclo[2.2.1]hept-2-yl]-4-(4-hydroxyphenylsulfanyl)-benzamide; N-[(1S,2R,4R)-7-azabicyclo[2.2.1]hept-2-yl]-4-(3-hydroxyphenylsulfanyl)-benzamide; N-[(1S,2R,4R)-7-azabicyclo[2.2.1]hept-2-yl]-4-(2-hydroxyphenylsulfanyl)-benzamide; N-[(1S,2R,4R)-7-azabicyclo[2.2.1]hept-2-yl]-4-(4-acetamidophenylsulfanyl)-benzamide; N-[(1S,2R,4R)-7-azabicyclo[2.2.1]hept-2-yl]-4-(3-acetamidophenylsulfanyl)-benzamide; N-[(1S,2R,4R)-7-azabicyclo[2.2.1]hept-2-yl]-4-(2-acetamidophenylsulfanyl)-benzamide; N-[(1S,2R,4R)-7-azabicyclo[2.2.1]hept-2-yl]-4-(thiophen-2-ylsulfanyl)-benzamide; N-[(1S,2R,4R)-7-azabicyclo[2.2.1]hept-2-yl]-4-(5-methylthiophen-2-ylsulfanyl)-benzamide; N-[(1S,2R,4R)-7-azabicyclo[2.2.1]hept-2-yl]-4-(5-chlorothiophen -2-ylsulfanyl)-benzamide; N-[(1S,2R,4R)-7-azabicyclo[2.2.1]hept-2-yl]-4-(5-methoxythiophen -2-ylsulfanyl)-benzamide; N-[(1S,2R,4R)-7-azabicyclo[2.2.1]hept-2-yl]-4-(5-trifluoromethylthiophen -2-ylsulfanyl)-benzamide; N-[(1S,2R,4R)-7-azabicyclo[2.2.1]hept-2-yl]-4-(5-acetylthiophen-2-ylsulfanyl)-benzamide; N-[(1S,2R,4R)-7-azabicyclo[2.2.1]hept-2-yl]-4-(5-acetaminothiophen-2-ylsulfanyl)-benzamide; N-[(1S,2R,4R)-7-azabicyclo[2.2.1]hept-2-yl]-4-(5-cyanothiophen -2-ylsulfanyl)-benzamide; N-[(1S,2R,4R)-7-azabicyclo[2.2.1]hept-2-yl]-4-(5-morpholin -4-yl-thiophen-2-ylsulfanyl)-benzamide; N-[(1S,2R,4R)-7-azabicyclo[2.2.1]hept-2-yl]-4-(4-methylthiophen-2-ylsulfanyl)-benzamide; N-[(1S,2R,4R)-7-azabicyclo[2.2.1]hept-2-yl]-4-(4-chlorothiophen-2-ylsulfanyl)-benzamide; N-[(1S,2R,4R)-7-azabicyclo[2.2.1]hept-2-yl]-4-(4-methoxythiophen -2-ylsulfanyl)-benzamide; N-[(1S,2R,4R)-7-azabicyclo[2.2.1]hept-2-yl]-4-(4-trifluoromethylthiophen-2-ylsulfanyl)-benzamide; N-[(1S,2R,4R)-7-azabicyclo[2.2.1]hept-2-yl]-4-(4-acetylthiophen-2-ylsulfanyl)-benzamide; N-[(1S,2R,4R)-7-azabicyclo[2.2.1]hept-2-yl]-4-(4-acetaminothiophen-2-ylsulfanyl)-benzamide; N-[(1S,2R,4R)-7-azabicyclo[2.2.1]hept-2-yl]-4-(4-cyanothiophen -2-ylsulfanyl)-benzamide; N-[(1S,2R,4R)-7-azabicyclo[2.2.1]hept-2-yl]-4-(4-morpholin -4-yl-thiophen-2-ylsulfanyl)-benzamide; N-[(1S,2R,4R)-7-azabicyclo[2.2.1]hept-2-yl]-4-(furan-2-ylsulfanyl)-benzamide; N-[(1S,2R,4R)-7-azabicyclo[2.2.1]hept-2-yl]-4-(5-methylfuran-2-ylsulfanyl)-benzamide; N-[(1S,2R,4R)-7-azabicyclo[2.2.1]hept-2-yl]-4-(5-chlorofuran-2-ylsulfanyl)-benzamide; N-[(1S,2R,4R)-7-azabicyclo[2.2.1]hept-2-yl]-4-(5-methoxyfuran-2-ylsulfanyl)-benzamide; N-[(1S,2R,4R)-7-azabicyclo[2.2.1]hept-2-yl]-4-(5-trifluoromethylfuran -2-ylsulfanyl)-benzamide; N-[(1S,2R,4R)-7-azabicyclo[2.2.1]hept-2-yl]-4-(5-acetylfuran -2-ylsulfanyl)-benzamide; N-[(1S,2R,4R)-7-azabicyclo[2.2.1]hept-2-yl]-4-(5-acetaminofuran-2-ylsulfanyl)-benzamide; N-[(1S,2R,4R)-7-azabicyclo[2.2.1]hept-2-yl]-4-(5-cyanofuran-2-ylsulfanyl)-benzamide; N-[(1S,2R,4R)-7-azabicyclo[2.2.1]hept-2-yl]-4-(5-morpholin-4-yl-furan-2-ylsulfanyl)-benzamide; N-[(1S,2R,4R)-7-azabicyclo[2.2.1]hept-2-yl]-4-(4-methylfuran-2-ylsulfanyl)-benzamide; N-[(1S,2R,4R)-7-azabicyclo[2.2.1]hept-2-yl]-4-(4-chlorofuran -2-ylsulfanyl)-benzamide; N-[(1S,2R,4R)-7-azabicyclo[2.2.1]hept-2-yl]-4-(4-methoxyfuran-2-ylsulfanyl)-benzamide; N-[(1S,2R,4R)-7-azabicyclo[2.2.1]hept-2-yl]-4-(4-trifluoromethylfuran-2-ylsulfanyl)-benzamide; N-[(1S,2R,4R)-7-azabicyclo[2.2.1]hept-2-yl]-4-(4-acetylfuran -2-ylsulfanyl)-benzamide; N-[(1S,2R,4R)-7-azabicyclo[2.2.1]hept-2-yl]-4-(4-acetaminofuran -2-ylsulfanyl)-benzamide; N-[(1S,2R,4R)-7-azabicyclo[2.2.1]hept-2-yl]-4-(4-cyanofuran-2-ylsulfanyl)-benzamide; N-[(1S,2R,4R)-7-azabicyclo[2.2.1]hept-2-yl]-4-(4-morpholin -4-yl-furan-2-ylsulfanyl)-benzamide; N-[(1S,2R,4R)-7-azabicyclo[2.2.1]hept-2-yl]-4-(oxazol-2-ylsulfanyl)-benzamide; N-[(1S,2R,4R)-7-azabicyclo[2.2.1]hept-2-yl]-4-(5-methyloxazol-2-ylsulfanyl)-benzamide; N-[(1S,2R,4R)-7-azabicyclo[2.2.1]hept-2-yl]-4-(5-chlorooxazol-2-ylsulfanyl)-benzamide; N-[(1S,2R,4R)-7-azabicyclo[2.2.1]hept-2-yl]-4-(5-methoxyoxazol -2-ylsulfanyl)-benzamide; N-[(1S,2R,4R)-7-azabicyclo[2.2.1]hept-2-yl]-4-(5-trifluoromethyloxazol -2-ylsulfanyl)-benzamide; N-[(1S,2R,4R)-7-azabicyclo[2.2.1]hept-2-yl]-4-(5-acetyloxazol-2-ylsulfanyl)-benzamide; N-[(1S,2R,4R)-7-azabicyclo[2.2.1]hept-2-yl]-4-(5-acetaminooxazol-2-ylsulfanyl)-benzamide; N-[(1S,2R,4R)-7-azabicyclo[2.2.1]hept-2-yl]-4-(5-cyanooxazol -2-ylsulfanyl)-benzamide; N-[(1S,2R,4R)-7-azabicyclo[2.2.1]hept-2-yl]-4-(5-morpholin-4-yl-oxazol -2-ylsulfanyl)-benzamide; N-[(1S,2R,4R)-7-azabicyclo[2.2.1]hept-2-yl]-4-(oxazol -2-ylsulfanyl)-benzamide; N-[(1S,2R,4R)-7-azabicyclo[2.2.1]hept-2-yl]-4-(4-methyloxazol-2-ylsulfanyl)-benzamide; N-[(1S,2R,4R)-7-azabicyclo[2.2.1]hept -2-yl]-4-(4-chlorooxazol-2-ylsulfanyl)-benzamide; N-[(1S,2R,4R)-7-azabicyclo[2.2.1]hept-2-yl]-4-(4-methoxyoxazol-2-ylsulfanyl)-benzamide; N-[(1S,2R,4R)-7-azabicyclo[2.2.1]hept-2-yl]-4-(4-trifluoromethyloxazol -2-ylsulfanyl)-benzamide; N-[(1S,2R,4R)-7-azabicyclo[2.2.1]hept-2-yl]-4-(4-acetyloxazol-2-ylsulfanyl)-benzamide; N-[(1S,2R,4R)-7-azabicyclo[2.2.1]hept-2-yl]-4-(4-acetaminooxazol-2-ylsulfanyl)-benzamide; N-[(1S,2R,4R)-7-azabicyclo[2.2.1]hept-2-yl]-4-(4-cyanooxazol-2-ylsulfanyl)-benzamide; N-[(1S,2R,4R)-7-azabicyclo[2.2.1]hept-2-yl]-4-(4-morpholin-4-yl-oxazol-2-ylsulfanyl)-benzamide; N-[(1S,2R,4R)-7-azabicyclo[2.2.1]hept-2-yl]-4-(oxazol-5-ylsulfanyl)-benzamide; N-[(1S,2R,4R)-7-azabicyclo[2.2.1]hept-2-yl]-4-(2-methyloxazol -5-ylsulfanyl)-benzamide; N-[(1S,2R,4R)-7-azabicyclo[2.2.1]hept-2-yl]-4-(2-chlorooxazol -5-ylsulfanyl)-benzamide; N-[(1S,2R,4R)-7-azabicyclo[2.2.1]hept-2-yl]-4-(2-methoxyoxazol -5-ylsulfanyl)-benzamide; N-[(1S,2R,4R)-7-azabicyclo[2.2.1]hept-2-yl]-4-(2-trifluoromethyloxazol-5-ylsulfanyl)-benzamide; N-[(1S,2R,4R)-7-azabicyclo[2.2.1]hept-2-yl]-4-(2-acetyloxazol-5-ylsulfanyl)-benzamide; N-[(1S,2R,4R)-7-azabicyclo[2.2.1]hept-2-yl]-4-(2-acetaminooxazol-5-ylsulfanyl)-benzamide; N-[(1S,2R,4R)-7-azabicyclo[2.2.1]hept-2-yl]-4-(2-cyanooxazol -5-ylsulfanyl)-benzamide; N-[(1S,2R,4R)-7-azabicyclo[2.2.1]hept-2-yl]-4-(2-morpholin-4-yl-oxazol -5-ylsulfanyl)-benzamide; N-[(1S,2R,4R)-7-azabicyclo[2.2.1]hept-2-yl]-4-(thiazol -2-ylsulfanyl)-benzamide; N-[(1S,2R,4R)-7-azabicyclo[2.2.1]hept-2-yl]-4-(5-methylthiazol-2-ylsulfanyl)-benzamide; N-[(1S,2R,4R)-7-azabicyclo[2.2.1]hept-2-yl]-4-(5-chlorothiazol-2-ylsulfanyl)-benzamide; N-[(1S,2R,4R)-7-azabicyclo[2.2.1]hept-2-yl]-4-(5-methoxythiazol-2-ylsulfanyl)-benzamide; N-[(1S,2R,4R)-7-azabicyclo[2.2.1]hept-2-yl]-4-(5-trifluoromethylthiazol -2-ylsulfanyl)-benzamide; N-[(1S,2R,4R)-7-azabicyclo[2.2.1]hept-2-yl]-4-(5-acetylthiazol-2-ylsulfanyl)-benzamide; N-[(1S,2R,4R)-7-azabicyclo[2.2.1]hept-2-yl]-4-(5-acetaminothiazol -2-ylsulfanyl)-benzamide; N-[(1S,2R,4R)-7-azabicyclo[2.2.1]hept-2-yl]-4-(5-cyanothiazol-2-ylsulfanyl)-benzamide; N-[(1S,2R,4R)-7-azabicyclo[2.2.1]hept-2-yl]-4-(5-morpholin-4-yl-thiazol-2-ylsulfanyl)-benzamide; N-[(1S,2R,4R)-7-azabicyclo[2.2.1]hept-2-yl]-4-(thiazol-2-ylsulfanyl)-benzamide; N-[(1S,2R,4R)-7-azabicyclo[2.2.1]hept-2-yl]-4-(4-methylthiazol -2-ylsulfanyl)-benzamide; N-[(1S,2R,4R)-7-azabicyclo[2.2.1]hept-2-yl]-4-(4-chlorothiazol -2-ylsulfanyl)-benzamide; N-[(1S,2R,4R)-7-azabicyclo[2.2.1]hept-2-yl]-4-(4-methoxythiazol -2-ylsulfanyl)-benzamide; N-[(1S,2R,4R)-7-azabicyclo[2.2.1]hept-2-yl]-4-(4-trifluoromethylthiazol-2-ylsulfanyl)-benzamide; N-[(1S,2R,4R)-7-azabicyclo[2.2.1]hept-2-yl]-4-(4-acetylthiazol-2-ylsulfanyl)-benzamide; N-[(1S,2R,4R)-7-azabicyclo[2.2.1]hept-2-yl]-4-(4-acetaminothiazol-2-ylsulfanyl)-benzamide; N-[(1S,2R,4R)-7-azabicyclo[2.2.1]hept-2-yl]-4-(4-cyanothiazol -2-ylsulfanyl)-benzamide; N-[(1S,2R,4R)-7-azabicyclo[2.2.1]hept-2-yl]-4-(4-morpholin-4-yl-thiazol -2-ylsulfanyl)-benzamide; N-[(1S,2R,4R)-7-azabicyclo[2.2.1]hept-2-yl]-4-(thiazol -5-ylsulfanyl)-benzamide; N-[(1S,2R,4R)-7-azabicyclo[2.2.1]hept-2-yl]-4-(2-methylthiazol-5-ylsulfanyl)-benzamide; N-[(1S,2R,4R)-7-azabicyclo[2.2.1]hept-2-yl]-4-(2-chlorothiazol-5-ylsulfanyl)-benzamide; N-[(1S,2R,4R)-7-azabicyclo[2.2.1]hept-2-yl]-4-(2-methoxythiazol-5-ylsulfanyl)-benzamide; N-[(1S,2R,4R)-7-azabicyclo[2.2.1]hept-2-yl]-4-(2-trifluoromethylthiazol -5-ylsulfanyl)-benzamide; N-[(1S,2R,4R)-7-azabicyclo[2.2.1]hept-2-yl]-4-(2-acetylthiazol-5-ylsulfanyl)-benzamide; N-[(1S,2R,4R)-7-azabicyclo[2.2.1]hept-2-yl]-4-(2-acetaminothiazol -5-ylsulfanyl)-benzamide; N-[(1S,2R,4R)-7-azabicyclo[2.2.1]hept-2-yl]-4-(2-cyanothiazol-5-ylsulfanyl)-benzamide; N-[(1S,2R,4R)-7-azabicyclo[2.2.1]hept-2-yl]-4-(2-morpholin-4-yl-thiazol-5-ylsulfanyl)-benzamide; N-[(1S,2R,4R)-7-azabicyclo[2.2.1]hept-2-yl]-4-([1,3,4]oxadiazol -2-ylsulfanyl)-benzamide; N-[(1S,2R,4R)-7-azabicyclo[2.2.1]hept-2-yl]-4-(5-methyl[1,3,4]oxadiazol-2-ylsulfanyl)-benzamide; N-[(1S,2R,4R)-7-azabicyclo[2.2.1]hept-2-yl]-4-(5-chloro[1,3,4]oxadiazol-2-ylsulfanyl)-benzamide; N-[(1S,2R,4R)-7-azabicyclo[2.2.1]hept-2-yl]-4-(5-methoxy[1,3,4]oxadiazol-2-ylsulfanyl)-benzamide; N-[(1S,2R,4R)-7-azabicyclo[2.2.1]hept-2-yl]-4-(5-trifluoromethyl[1,3,4]oxadiazol-2-ylsulfanyl)-benzamide; N-[(1S,2R,4R)-7-azabicyclo[2.2.1]hept-2-yl]-4-(5-acetyl[1,3,4]oxadiazol-2-ylsulfanyl)-benzamide; [(1S,2R,4R)-7-azabicyclo[2.2.1]hept-2-yl]-4-(5-acetamino[1,3,4]oxadiazol-2-ylsulfanyl)-benzamide; N-[(1S,2R,4R)-7-azabicyclo[2.2.1]hept-2-yl]-4-(5-cyano[1,3,4]oxadiazol-2-ylsulfanyl)-benzamide; N-[(1S,2R,4R)-7-azabicyclo[2.2.1]hept-2-yl]-4-(5-morpholin-4-yl-[1,3,4]oxadiazol-2-ylsulfanyl)-benzamide; N-[(1S,2R,4R)-7-azabicyclo[2.2.1]hept-2-yl]-4-([1,3,4]thiadiazol -2-ylsulfanyl)-benzamide; N-[(1S,2R,4R)-7-azabicyclo[2.2.1]hept-2-yl]-4-(5-methyl[1,3,4]thiadiazol-2-ylsulfanyl)-benzamide; N-[(1S,2R,4R)-7-azabicyclo[2.2.1]hept-2-yl]-4-(5-chloro[1,3,4]thiadiazol-2-ylsulfanyl)-benzamide; N-[(1S,2R,4R)-7-azabicyclo[2.2.1]hept-2-yl]-4-(5-methoxy[1,3,4]thiadiazol -2-ylsulfanyl)-benzamide; N-[(1S,2R,4R)-7-azabicyclo[2.2.1]hept-2-yl]-4-(5-trifluoromethyl[1,3,4]thiadiazol-2-ylsulfanyl)-benzamide; N-[(1S,2R,4R)-7-azabicyclo[2.2.1]hept-2-yl]-4-(5-acetyl[1,3,4]thiadiazol-2-ylsulfanyl)-benzamide; N-[(1S,2R,4R)-7-azabicyclo[2.2.1]hept-2-yl]-4-(5-acetamino[1,3,4]thiadiazol -2-ylsulfanyl)-benzamide; N-[(1S,2R,4R)-7-azabicyclo[2.2.1]hept-2-yl]-4-(5-cyano[1,3,4]thiadiazol-2-ylsulfanyl)-benzamide; N-[(1S,2R,4R)-7-azabicyclo[2.2.1]hept-2-yl]-4-(5-morpholin-4-yl-[1,3,4]thiadiazol-2-ylsulfanyl)-benzamide; N-[(1S,2R,4R)-7-azabicyclo[2.2.1]hept-2-yl]-4-(pyrrol-2-yloxy)-benzamide; N-[(1S,2R,4R)-7-azabicyclo[2.2.1]hept-2-yl]-4-(5-methylpyrrol -2-yloxy)-benzamide; N-[(1S,2R,4R)-7-azabicyclo[2.2.1]hept-2-yl]-4-(5-chloropyrrol -2-yloxy)-benzamide; N-[(1S,2R,4R)-7-azabicyclo[2.2.1]hept-2-yl]-4-(5-methoxypyrrol-2-yloxy)-benzamide; N-[(1S,2R,4R)-7-azabicyclo[2.2.1]hept-2-yl]-4-(5-trifluoromethylpyrrol-2-yloxy)-benzamide; N-[(1S,2R,4R)-7-azabicyclo[2.2.1]hept-2-yl]-4-(5-acetylpyrrol-2-yloxy)-benzamide; N-[(1S,2R,4R) -7-azabicyclo[2.2.1]hept-2-yl]-4-(5-acetaminopyrrol-2-yloxy)-benzamide; N-[(1S,2R,4R)-7-azabicyclo[2.2.1]hept-2-yl]-4-(5-cyanopyrrol-2-yloxy)-benzamide; N-[(1S,2R,4R) -7-azabicyclo[2.2.1]hept-2-yl]-4-(5-morpholin-4-yl-pyrrol-2-yloxy)-benzamide; N-[(1S,2R,4R)-7-azabicyclo[2.2.1]hept-2-yl]-4-(3H-imidazol-4-yloxy)-benzamide; N-[(1S,2R,4R)-7-azabicyclo[2.2.1]hept-2-yl]-4-(2-methyl-3H-imidazol -4-yloxy)-benzamide; N-[(1S,2R,4R)-7-azabicyclo[2.2.1]hept-2-yl]-4-(2-chloro-3H-imidazol -4-yloxy)-benzamide; N-[(1S,2R,4R)-7-azabicyclo[2.2.1]hept-2-yl]-4-(2-methoxy -3H-imidazol-4-yloxy)-benzamide; N-[(1S,2R,4R)-7-azabicyclo[2.2.1]hept-2-yl]-4-(2-trifluoromethyl-3H-imidazol-4-yloxy)-benzamide; N-[(1S,2R,4R)-7-azabicyclo[2.2.1]hept-2-yl]-4-(2-acetyl-3H-imidazol-4-yloxy)-benzamide; N-[(1S,2R,4R)-7-azabicyclo[2.2.1]hept-2-yl]-4-(2-acetamino-3H-imidazol -4-yloxy)-benzamide; N-[(1S,2R,4R)-7-azabicyclo[2.2.1]hept-2-yl]-4-(2-cyano-3H-imidazol -4-yloxy)-benzamide; N-[(1S,2R,4R)-7-azabicyclo[2.2.1]hept-2-yl]-4-(2-morpholin -4-yl-3H-imidazol-4-yloxy)-benzamide; N-[(1S,2R,4R)-7-azabicyclo[2.2.1]hept-2-yl]-4-(isoxazol-2-yloxy)-benzamide; N-[(1S,2R,4R)-7-azabicyclo[2.2.1]hept-2-yl]-4-(5-methylisoxazol-3-yloxy)-benzamide; N-[(1S,2R,4R)-7-azabicyclo[2.2.1]hept-2-yl]-4-(5-chloroisoxazol-3-yloxy)-benzamide; N-[(1S,2R,4R)-7-azabicyclo[2.2.1]hept-2-yl]-4-(5-methoxyisoxazol-3-yloxy)-benzamide; N-[(1S,2R,4R)-7-azabicyclo[2.2.1]hept-2-yl]-4-(5-trifluoromethylisoxazol-3-yloxy)-benzamide; N-[(1S,2R,4R)-7-azabicyclo[2.2.1]hept-2-yl]-4-(5-acetylisoxazol -3-yloxy)-benzamide; N-[(1S,2R,4R)-7-azabicyclo[2.2.1]hept-2-yl]-4-(5-acetaminoisoxazol -3-yloxy)-benzamide; N-[(1S,2R,4R)-7-azabicyclo[2.2.1]hept-2-yl]-4-(5-cyanoisoxazol -3-yloxy)-benzamide; N-[(1S,2R,4R)-7-azabicyclo[2.2.1]hept-2-yl]-4-(5-morpholin-4-yl-isoxazol-3-yloxy)-benzamide; N-[(1S,2R,4R)-7-azabicyclo[2.2.1]hept-2-yl]-4-(isothiazol-2-yloxy)-benzamide; N-[(1S,2R,4R)-7-azabicyclo[2.2.1]hept-2-yl]-4-(5-methylisothiazol-3-yloxy)-benzamide; N-[(1S,2R,4R)-7-azabicyclo[2.2.1]hept-2-yl]-4-(5-chloroisothiazol-3-yloxy)-benzamide; N-[(1S,2R,4R)-7-azabicyclo[2.2.1]hept-2-yl]-4-(5-methoxyisothiazol-3-yloxy)-benzamide; N-[(1S,2R,4R)-7-azabicyclo[2.2.1]hept-2-yl]-4-(5-trifluoromethylisothiazol -3-yloxy)-benzamide; N-[(1S,2R,4R)-7-azabicyclo[2.2.1]hept-2-yl]-4-(5-acetylisothiazol -3-yloxy)-benzamide; N-[(1S,2R,4R)-7-azabicyclo[2.2.1]hept-2-yl]-4-(5-acetaminoisothiazol-3-yloxy)-benzamide; N-[(1S,2R,4R)-7-azabicyclo[2.2.1]hept-2-yl]-4-(5-cyanoisothiazol-3-yloxy)-benzamide; N-[(1S,2R,4R)-7-azabicyclo[2.2.1]hept-2-yl]-4-(5-morpholin-4-yl-isothiazol-3-yloxy)-benzamide; N-[(1S,2R,4R)-7-azabicyclo[2.2.1]hept-2-yl]-4-(pyrrol-2-ylsulfanyl)-benzamide; N-[(1S,2R,4R)-7-azabicyclo[2.2.1]hept-2-yl]-4-(5-methylpyrrol -2-ylsulfanyl)-benzamide; N-[(1S,2R,4R)-7-azabicyclo[2.2.1]hept-2-yl]-4-(5-chloropyrrol -2-ylsulfanyl)-benzamide; N-[(1S,2R,4R)-7-azabicyclo[2.2.1]hept-2-yl]-4-(5-methoxypyrrol-2-ylsulfanyl)-benzamide; N-[(1S,2R,4R)-7-azabicyclo[2.2.1]hept-2-yl]-4-(5-trifluoromethylpyrrol-2-ylsulfanyl)-benzamide; N-[(1S,2R,4R)-7-azabicyclo[2.2.1]hept-2-yl]-4-(5-acetylpyrrol -2-ylsulfanyl)-benzamide; N-[(1S,2R,4R)-7-azabicyclo[2.2.1]hept-2-yl]-4-(5-acetaminopyrrol -2-ylsulfanyl)-benzamide; N-[(1S,2R,4R)-7-azabicyclo[2.2.1]hept-2-yl]-4-(5-cyanopyrrol -2-ylsulfanyl)-benzamide; N-[(1S,2R,4R)-7-azabicyclo[2.2.1]hept-2-yl]-4-(5-morpholin-4-yl-pyrrol-2-ylsulfanyl)-benzamide; N-[(1S,2R,4R)-7-azabicyclo[2.2.1]hept-2-yl]-4-(3H-imidazol-4-ylsulfanyl)-benzamide; N-[(1S,2R,4R)-7-azabicyclo[2.2.1]hept-2-yl]-4-(2-methyl-3H-imidazol-4-ylsulfanyl)-benzamide; N-[(1S,2R,4R)-7-azabicyclo[2.2.1]hept-2-yl]-4-(2-chloro-3H-imidazol -4-ylsulfanyl)-benzamide; N-[(1S,2R,4R)-7-azabicyclo[2.2.1]hept-2-yl]-4-(2-methoxy-3H-imidazol -4-ylsulfanyl)-benzamide; N-[(1S,2R,4R)-7-azabicyclo[2.2.1]hept-2-yl]-4-(2-trifluoromethyl-3H-imidazol-4-ylsulfanyl)-benzamide; N-[(1S,2R,4R)-7-azabicyclo[2.2.1]hept-2-yl]-4-(2-acetyl-3H-imidazol-4-ylsulfanyl)-benzamide; N-[(1S,2R,4R)-7-azabicyclo[2.2.1]hept-2-yl]-4-(2-acetamino-3H-imidazol -4-ylsulfanyl)-benzamide; N-[(1S,2R,4R)-7-azabicyclo[2.2.1]hept-2-yl]-4-(2-cyano -3H-imidazol-4-ylsulfanyl)-benzamide; N-[(1S,2R,4R)-7-azabicyclo[2.2.1]hept-2-yl]-4-(2-morpholin-4-yl-3H-imidazol-4-ylsulfanyl)-benzamide; N-[(1S,2R,4R) -7-azabicyclo[2.2.1]hept-2-yl]-4-(isoxazol-2-ylsulfanyl)-benzamide; N-[(1S,2R,4R)-7-azabicyclo[2.2.1]hept-2-yl]-4-(5-methylisoxazol-3-ylsulfanyl)-benzamide; N-[(1S,2R,4R)-7-azabicyclo[2.2.1]hept-2-yl]-4-(5-chloroisoxazol-3-ylsulfanyl)-benzamide; N-[(1S,2R,4R)-7-azabicyclo[2.2.1]hept-2-yl]-4-(5-methoxyisoxazol -3-ylsulfanyl)-benzamide; N-[(1S,2R,4R)-7-azabicyclo[2.2.1]hept-2-yl]-4-(5-trifluoromethylisoxazol -3-ylsulfanyl)-benzamide; N-[(1S,2R,4R)-7-azabicyclo[2.2.1]hept-2-yl]-4-(5-acetylisoxazol-3-ylsulfanyl)-benzamide; N-[(1S,2R,4R)-7-azabicyclo[2.2.1]hept-2-yl]-4-(5-acetaminoisoxazol-3-ylsulfanyl)-benzamide; N-[(1S,2R,4R)-7-azabicyclo[2.2.1]hept-2-yl]-4-(5-cyanoisoxazol -3-ylsulfanyl)-benzamide; N-[(1S,2R,4R)-7-azabicyclo[2.2.1]hept-2-yl]-4-(5-morpholin-4-yl-isoxazol -3-ylsulfanyl)-benzamide; N-[(1S,2R,4R)-7-azabicyclo[2.2.1]hept-2-yl]-4-(isothiazol -2-ylsulfanyl)-benzamide; N-[(1S,2R,4R)-7-azabicyclo[2.2.1]hept-2-yl]-4-(5-methylisothiazol-3-ylsulfanyl)-benzamide; N-[(1S,2R,4R)-7-azabicyclo[2.2.1]hept-2-yl]-4-(5-chloroisothiazol-3-ylsulfanyl)-benzamide; N-[(1S,2R,4R)-7-azabicyclo[2.2.1]hept-2-yl]-4-(5-methoxyisothiazol -3-ylsulfanyl)-benzamide; N-[(1S,2R,4R)-7-azabicyclo[2.2.1]hept-2-yl]-4-(5-trifluoromethylisothiazol -3-ylsulfanyl)-benzamide; N-[(1S,2R,4R)-7-azabicyclo[2.2.1]hept-2-yl]-4-(5-acetylisothiazol-3-ylsulfanyl)-benzamide; N-[(1S,2R,4R)-7-azabicyclo[2.2.1]hept-2-yl]-4-(5-acetaminoisothiazol -3-ylsulfanyl)-benzamide; N-[(1S,2R,4R)-7-azabicyclo[2.2.1]hept-2-yl]-4-(5-cyanoisothiazol -3-ylsulfanyl)-benzamide; N-[(1S,2R,4R)-7-azabicyclo[2.2.1]hept-2-yl]-4-(5-morpholin -4-yl-isothiazol-3-ylsulfanyl)-benzamide; N-[(1S,2R,4R)-7-azabicyclo[2.2.1]hept-2-yl]-4-(6-methylpyridin-3-yloxy)-benzamide; N-[(1S,2R,4R)-7-azabicyclo[2.2.1]hept-2-yl]-4-(6-chloropyridin-3-yloxy)-benzamide; N-[(1S,2R,4R)-7-azabicyclo[2.2.1]hept-2-yl]-4-(6-methoxypyridin-3-yloxy)-benzamide; N-[(1S,2R,4R)-7-azabicyclo[2.2.1]hept-2-yl]-4-(6-trifluoromethylpyridin -3-yloxy)-benzamide; N-[(1S,2R,4R)-7-azabicyclo[2.2.1]hept-2-yl]-4-(6-acetylpyridin -3-yloxy)-benzamide; N-[(1S,2R,4R)-7-azabicyclo[2.2.1]hept-2-yl]-4-(6-acetaminopyridin -3-yloxy)-benzamide; N-[(1S,2R,4R)-7-azabicyclo[2.2.1]hept-2-yl]-4-(6-cyanopyridin-3-yloxy)-benzamide; N-[(1S,2R,4R)-7-azabicyclo[2.2.1]hept -2-yl]-4-(6-morpholin-4-yl-pyridin-3-yloxy)-benzamide; N-[(1S,2R,4R)-7-azabicyclo[2.2.1]hept-2-yl]-4-(2-methylpyridin-4-yloxy)-benzamide; N-[(1S,2R,4R)-7-azabicyclo[2.2.1]hept-2-yl]-4-(2-chloropyridin-4-yloxy)-benzamide; N-[(1S, 2R,4R)-7-azabicyclo[2.2.1]hept-2-yl]-4-(2-methoxypyridin-4-yloxy)-benzamide; N-[(1S,2R,4R)-7-azabicyclo[2.2.1]hept-2-yl]-4-(2-trifluoromethylpyridin -4-yloxy)-benzamide; N-[(1S,2R,4R)-7-azabicyclo[2.2.1]hept-2-yl]-4-(2-acetylpyridin -4-yloxy)-benzamide; N-[(1S,2R,4R)-7-azabicyclo[2.2.1]hept-2-yl]-4-(2-acetaminopyridin -4-yloxy)-benzamide; N-[(1S,2R,4R)-7-azabicyclo[2.2.1]hept-2-yl]-4-(2-cyanopyridin-4-yloxy)-benzamide; N-[(1S,2R,4R)-7-azabicyclo[2.2.1]hept -2-yl]-4-(2-morpholin-4-yl-pyridin-4-yloxy)-benzamide; N-[(1S,2R,4R)-7-azabicyclo[2.2.1]hept-2-yl]-4-(5-methylpyridin-2-yloxy)-benzamide; N-[(1S,2R,4R)-7-azabicyclo[2.2.1]hept-2-yl]-4-(5-chloropyridin-2-yloxy)-benzamide; N-[(1S,2R,4R)-7-azabicyclo[2.2.1]hept-2-yl]-4-(5-methoxypyridin-2-yloxy)-benzamide; N-[(1S,2R,4R)-7-azabicyclo[2.2.1]hept-2-yl]-4-(5-trifluoromethylpyridin -2-yloxy)-benzamide; N-[(1S,2R,4R)-7-azabicyclo[2.2.1]hept-2-yl]-4-(5-acetylpyridin -2-yloxy)-benzamide; N-[(1S,2R,4R)-7-azabicyclo[2.2.1]hept-2-yl]-4-(5-acetaminopyridin -2-yloxy)-benzamide; N-[(1S,2R,4R)-7-azabicyclo[2.2.1]hept-2-yl]-4-(5-cyanopyridin-2-yloxy)-benzamide; N-[(1S,2R,4R)-7-azabicyclo[2.2.1 ]hept-2-yl]-4-(5-morpholin-4-yl-pyridin-2-yloxy)-benzamide; N-[(1S,2R,4R)-7-azabicyclo[2.2.1]hept-2-yl]-4-(4-methylpyridin-2-yloxy)-benzamide; N-[(1S,2R,4R)-7-azabicyclo[2.2.1]hept-2-yl]-4-(4-chloropyridin-2-yloxy)-benzamide; N-[(1S,2R,4R)-7-azabicyclo[2.2.1]hept-2-yl]-4-(4-methoxypyridin-2-yloxy)-benzamide; N-[(1S,2R,4R)-7-azabicyclo[2.2.1]hept-2-yl]-4-(4-trifluoromethylpyridin -2-yloxy)-benzamide; N-[(1S,2R,4R)-7-azabicyclo[2.2.1]hept-2-yl]-4-(4-acetylpyridin -2-yloxy)-benzamide; N-[(1S,2R,4R)-7-azabicyclo[2.2.1]hept-2-yl]-4-(4-acetaminopyridin -2-yloxy)-benzamide; N-[(1S,2R,4R)-7-azabicyclo[2.2.1]hept-2-yl]-4-(4-cyanopyridin-2-yloxy)-benzamide; N-[(1S,2R,4R)-7-azabicyclo[2.2.1]hept -2-yl]-4-(4-morpholin-4-yl-pyridin-2-yloxy)-benzamide; N-[(1S,2R,4R)-7-azabicyclo[2.2.1]hept-2-yl]-4-(6-methylpyridin-2-yloxy)-benzamide; N-[(1S,2R,4R)-7-azabicyclo[2.2.1]hept-2-yl]-4-(6-chloropyridin-2-yloxy)-benzamide; N-[(1S,2R,4R)-7-azabicyclo[2.2.1]hept-2-yl]-4-(6-methoxypyridin-2-yloxy)-benzamide; N-[(1S,2R,4R)-7-azabicyclo[2.2.1]hept-2-yl]-4-(6-trifluoromethylpyridin -2-yloxy)-benzamide; N-[(1S,2R,4R)-7-azabicyclo[2.2.1]hept-2-yl]-4-(6-acetylpyridin -2-yloxy)-benzamide; N-[(1S,2R,4R)-7-azabicyclo[2.2.1]hept-2-yl]-4-(6-acetaminopyridin -2-yloxy)-benzamide; N-[(1S,2R,4R)-7-azabicyclo[2.2.1]hept-2-yl]-4-(6-cyanopyridin-2-yloxy)-benzamide; N-[(1S,2R,4R)-7-azabicyclo[2.2.1]hept -2-yl]-4-(6-morpholin-4-yl-pyridin-2-yloxy)-benzamide, N-[(1S,2R,4R) -7-azabicyclo[2.2.1]hept-2-yl]-4-(5-methylpyrdin-3-yloxy)-benzamide; N-[(1S,2R,4R)-7-azabicyclo[2.2.1]hept-2-yl]-4-(5-chloropyridin-3-yloxy)-benzamide; N-[(1S,2R,4R)-7-azabicyclo[2.2.]hept-2-yl]-4-(5-methoxypyridin-3-yloxy)-benzamide; N-[(1S,2R,4R)-7-azabicyclo[2.2.1]hept-2-yl]-4-(5-trifluoromethylpyridin -3-yloxy)-benzamide; N-[(1S,2R,4R)-7-azabicyclo[2.2.1]hept-2-yl]-4-(5-acetylpyridin -3-yloxy)-benzamide; N-[(1S,2R,4R)-7-azabicyclo[2.2.1]hept-2-yl]-4-(5-acetaminopyridin -3-yloxy)-benzamide; N-[(1S,2R,4R)-7-azabicyclo[2.2.1]hept-2-yl]-4-(5-cyanopyridin-3-yloxy)-benzamide; N-[(1S,2R,4R)-7-azabicyclo[2.2.1]hept -2-yl]-4-(5-morpholin-4-yl-pyridin-3-yloxy)-benzamide; N-[(1S,2R,4R)-7-azabicyclo[2.2.1]hept-2-yl]-4-(2-chloropyridin-3-yloxy)-benzamide; N-[(1S,2R,4R) -7-azabicyclo[2.2.1]hept-2-yl]-4-(4-chloropyridin-3-yloxy)-benzamide; N-[(1S,2R,4R)-7-azabicyclo[2.2.1]hept-2-yl]-4-(3-chloropyridin-4-yloxy)-benzamide; N-[(1S,2R,4R)-7-azabicyclo[2.2.1]hept-2-yl]-4-(3-chloropyridin-2-yloxy)-benzamide; N-[(1S,2R,4R)-7-azabicyclo[2.2.1]hept-2-yl]-4-(6-methylpyridin -3-ylsulfanyl)-benzamide; N-[(1S,2R,4R)-7-azabicyclo[2.2.1]hept-2-yl]-4-(6-chloropyridin -3-ylsulfanyl)-benzamide; N-[(1S,2R,4R)-7-azabicyclo[2.2.1]hept-2-yl]-4-(6-methoxypyridin -3-ylsulfanyl)-benzamide; N-[(1S,2R,4R)-7-azabicyclo[2.2.1]hept-2-yl]-4-(6-trifluoromethylpyridin-3-ylsulfanyl)-benzamide; N-[(1S,2R,4R)-7-azabicyclo[2.2.1]hept-2-yl]-4-(6-acetylpyridin-3-ylsulfanyl)-benzamide; N-[(1S,2R,4R)-7-azabicyclo[2.2.1]hept-2-yl]-4-(6-acetaminopyridin-3-ylsulfanyl)-benzamide; N-[(1S,2R,4R)-7-azabicyclo[2.2.1]hept-2-yl]-4-(6-cyanopyridin -3-ylsulfanyl)-benzamide; N-[(1S,2R,4R)-7-azabicyclo[2.2.1]hept-2-yl]-4-(6-morpholin-4-yl-pyridin -3-ylsulfanyl)-benzamide; N-[(1S,2R,4R)-7-azabicyclo[2.2.1]hept-2-yl]-4-(2-methylpyridin -4-ylsulfanyl)-benzamide; N-[(1S,2R,4R)-7-azabicyclo[2.2.1]hept-2-yl]-4-(2-chloropyridin-4-ylsulfanyl)-benzamide; N-[(1S,2R,4R)-7-azabicyclo[2.2.1]hept-2-yl]-4-(2-methoxypyridin-4-ylsulfanyl)-benzamide; N-[(1S,2R,4R)-7-azabicyclo[2.2.1]hept-2-yl]-4-(2-trifluoromethylpyridin -4-ylsulfanyl)-benzamide; N-[(1S,2R,4R)-7-azabicyclo[2.2.1]hept-2-yl]-4-(2-acetylpyridin -4-ylsulfanyl)-benzamide; N-[(1S,2R,4R)-7-azabicyclo[2.2.1]hept-2-yl]-4-(2-acetaminopyridin -4-ylsulfanyl)-benzamide; N-[(1S,2R,4R)-7-azabicyclo[2.2.1]hept -2-yl]-4-(2-cyanopyridin-4-ylsulfanyl)-benzamide; N-[(1S,2R,4R) -7-azabicyclo[2.2.1])hept-2-yl]-4-(2-morpholin-4-yl-pyfidin-4-ylsulfanyl)-benzamide; N-[(1S,2R,4R)-7-azabicyclo[2.2.1]hept-2-yl]-4-(5-methylpyridin -2-ylsulfanyl)-benzamide; N-[(1S,2R,4R)-7-azabicyclo[2.2.1]hept-2-yl]-4-(5-chloropyridin -2-ylsulfanyl)-benzamide; N-[(1S,2R,4R)-7-azabicyclo[2.2.1]hept-2-yl]-4-(5-methoxypyridin -2-ylsulfanyl)-benzamide; N-[(1S,2R,4R)-7-azabicyclo[2.2.1]hept-2-yl]-4-(5-trifluoromethylpyridin-2-ylsulfanyl)-benzamide; N-[(1S,2R,4R)-7-azabicyclo[2.2.1]hept-2-yl]-4-(5-acetylpyridin-2-ylsulfanyl)-benzamide; N-[(1S 2R, 4R)-7-azabicyclo[2.2.1]hept-2-yl]-4-(5-acetaminopyridin-2-ylsulfanyl)-benzamide; N-[(1S,2R,4R)-7-azabicyclo[2.2.1]hept-2-yl]-4-(5-cyanopyridin -2-ylsulfanyl)-benzamide; N-[(1S,2R,4R)-7-azabicyclo[2.2.1]hept-2-yl]-4-(5-morpholin -4-yl-pyridin -2-ylsulfanyl)-benzamide; N-[(1S,2R,4R)-7-azabicyclo[2.2.1]hept-2-yl]-4-(4-methylpyridin -2-ylsulfanyl)-benzamide; N-[(1S,2R,4R)-7-azabicyclo[2.2.1]hept-2-yl]-4-(4-chloropyridin-2-ylsulfanyl)-benzamide; N-[(1S,2R,4R)-7-azabicyclo[2.2.1]hept-2-yl]-4-(4-methoxypyridin-2-ylsulfanyl)-benzamide; N-[(1S,2R,4R)-7-azabicyclo[2.2.1]hept-2-yl]-4-(4-trifluoromethylpyridin -2-ylsulfanyl)-benzamide; N-[(1S,2R,4R)-7-azabicyclo[2.2.1]hept-2-yl]-4-(4-acetylpyridin -2-ylsulfanyl)-benzamide; N-[(1S,2R,4R)-7-azabicyclo[2.2.1]hept-2-yl]-4-(4-acetaminopyridin -2-ylsulfanyl)-benzamide; N-[(1S,2R,4R)-7-azabicyclo[2.2.1]hept-2-yl]-4-(4-cyanopyridin-2-ylsulfanyl)-benzamide; N-[(1S,2R,4R)-7-azabicyclo[2.2.1]hept-2-yl]-4-(4-morpholin-4-yl-pyridin-2-ylsulfanyl)-benzamide; N-[(1S,2R,4R)-7-azabicyclo[2.2.1]hept-2-yl]-4-(6-methylpyridin -2-ylsulfanyl)-benzamide; N-[(1S,2R,4R)-7-azabicyclo[2.2.1]hept-2-yl]-4-(6-chloropyridin -2-ylsulfanyl)-benzamide; N-[(1S,2R,4R)-7-azabicyclo[2.2.1]hept-2-yl]-4-(6-methoxypyridin -2-ylsulfanyl)-benzamide; N-[(1S,2R,4R)-7-azabicyclo[2.2.1]hept-2-yl]-4-(6-trifluoromethylpyridin-2-ylsulfanyl)-benzamide; N-[(1S,2R,4R)-7-azabicyclo[2.2.1]hept-2-yl]-4-(6-acetylpyridin-2-ylsulfanyl)-benzamide; N-[(1S,2R,4R)-7-azabicyclo[2.2.1]hept-2-yl]-4-(6-acetaminopyridin-2-ylsulfanyl)-benzamide; N-[(1S,2R,4R)-7-azabicyclo[2.2.1]hept-2-yl]-4-(6-cyanopyridin -2-ylsulfanyl)-benzamide; N-[(1S,2R,4R)-7-azabicyclo[2.2.1]hept-2-yl]-4-(6-morpholin-4-yl-pyridin -2-ylsulfanyl)-benzamide; N-[(1S,2R,4R)-7-azabicyclo[2.2.1]hept-2-yl]-4-(5-methylpyridin -3-ylsulfanyl)-benzamide; N-[(1S,2R,4R)-7-azabicyclo[2.2.1]hept-2-yl]-4-(5-chloropyridin-3-ylsulfanyl)-benzamide; N-[(1S,2R,4R)-7-azabicyclo[2.2.1]hept-2-yl]-4-(5-methoxypyridin-3-ylsulfanyl)-benzamide; N-[(1S,2R,4R)-7-azabicyclo[2.2.1]hept-2-yl]-4-(5-trifluoromethylpyridin -3-ylsulfanyl)-benzamide; N-[(1S,2R,4R)-7-azabicyclo[2.2.1]hept-2-yl]-4-(5-acetylpyridin -3-ylsulfanyl)-benzamide; N-[(1S,2R,4R)-7-azabicyclo[2.2.1]hept-2-yl]-4-(5-acetaminopyridin -3-ylsulfanyl)-benzamide; N-[(1S,2R,4R)-7-azabicyclo[2.2.1]hept-2-yl]-4-(5-cyanopyridin-3-ylsulfanyl)-benzamide; N-[(1S,2R,4R)-7-azabicyclo[2.2.1]hept-2-yl]-4-(5-morpholin-4-yl-pyridin-3-ylsulfanyl)-benzamide; N-[(1S,2R,4R)-7-azabicyclo[2.2.1]hept-2-yl]-4-(2-chloropyridin -3-ylsulfanyl)-benzamide; N-[(1S,2R,4R)-7-azabicyclo[2.2.1]hept-2-yl]-4-(4-chloropyridin -3-ylsulfanyl)-benzamide; N-[(1S,2R,4R)-7-azabicyclo[2.2.1]hept-2-yl]-4-(3-chloropyridin -4-ylsulfanyl)-benzamide; or N-[(1S,2R,4R)-7-azabicyclo[2.2.1]hept -2-yl]-4-(3-chloropyridin-2-ylsulfanyl)-benzamide.

The compound of Formula I or formula A-L-B, where the compound is any one or more or combination of the following as the free base, or a pharmaceutically acceptable salt thereof: N-[(1S,2R,4R)-7-azabicyclo[2.2.1]hept-2-yl]-2-naphthamide; N-[(1S,2R,4R)-7-azabicyclo[2.2.1]hept-2-yl]-7-methoxy-2-naphthamide; or a pharmaceutically acceptable salt thereof.

The compound of Formula I or formula A-L-B, where the compound is any one or more or combination of the following as the free base, or a pharmaceutically acceptable salt thereof: N-[(1S,2R,4R)-7-azabicyclo[2.2.1]hept-2-yl]-7-methyl -2-naphthamide; N-[(1S,2R,4R)-7-azabicyclo[2.2.1]hept-2-yl]-7-hydroxy-2-naphthamide; N-[(1S,2R,4R)-7-azabicyclo[2.2.1]hept-2-yl]-7-methoxy-2-naphthamide; N-[(1S,2R,4R)-7-azabicyclo[2.2.1]hept-2-yl]-7-mercapto-2-naphthamide; N-[(1S,2R,4R)-7-azabicyclo[2.2.1]hept-2-yl]-7-methylthio-2-naphthamide; 7-amino-N-[(1S,2R,4R)-7-azabicyclo[2.2.1]hept-2-yl]-2-naphthamide; N-[(1S,2R,4R)-7-azabicyclo[2.2.1]hept-2-yl]-7-methylamino-2-naphthamide; N-[(1S,2R,4R)-7-azabicyclo[2.2.1]hept-2-yl]-7-fluoro-2-naphthamide; N-[(1S,2R,4R)-7-azabicyclo[2.2.1]hept-2-yl]-7-cyano-2-naphthamide; N-[(1S,2R,4R)-7-azabicyclo[2.2.1]hept-2-yl]-7-chloro-2-naphthamide; N-[(1S,2R,4R)-7-azabicyclo[2.2.1]hept-2-yl]-7-bromo-2-naphthamide; N-[(1S,2R,4R) -7-azabicyclo[2.2.1]hept-2-yl]-7-iodo-2-naphthamide; N-[(1S,2R,4R)-7-azabicyclo[2.2.1]hept-2-yl]-7-nitro-2-naphthamide; N-[(1S,2R,4R)-7-azabicyclo[2.2.1]hept-2-yl]-7-ethynyl-2-naphthamide; N-[(1S,2R,4R) -7-azabicyclo[2.2.1]hept-2-yl]-7-trifluoromethyl-2-naphthamide; N-[(1S,2R,4R)-7-azabicyclo[2.2.1]hept-2-yl]-7-prop-1-ynyl-2-naphthamide; N-[(1S,2R,4R) -7-azabicyclo[2.2.1]hept-2-yl]-7-ethenyl-2-naphthamide; N-[(1S,2R,4R)-7-azabicyclo[2.2.1]hept-2-yl]-7-(3-hydroxyprop-1-ynyl)-2-naphthamide; 7-(acetylamino)-N-[(1S,2R,4R)-7-azabicyclo[2.2.1]hept-2-yl]-2-naphthamide; N-[(1S,2R,4R)-7-azabicyclo[2.2.1]hept-2-yl]-7-(formylamino)-2-naphthamide; N-[(1S,2R,4R)-7-azabicyclo[2.2.1]hept-2-yl]-5-methyl-2-naphthamide; N-[(1S,2R,4R) -7-azabicyclo[2.2.1]hept-2-yl]-5-hydroxy-2-naphthamide; N-[(1S,2R,4R)-7-azabicyclo[2.2.1]hept-2-yl]-5-methoxy-2-naphthamide; N-[(1S,2R,4R)-7-azabicyclo[2.2.1]hept-2-yl]-5-mercapto-2-naphthamide; N-[(1S,2R,4R)-7-azabicyclo[2.2.1]hept-2-yl]-5-methylthio-2-naphthamide; 5-Amino-N-[(1S,2R,4R) -7-azabicyclo[2.2.1]hept-2-yl]-2-naphthamide; N-[(1S,2R,4R)-7-azabicyclo[2.2.1]hept-2-yl]-5-methylamino-2-naphthamide; N-[(1S,2R,4R)-7-azabicyclo[2.2.1]hept-2-yl]-5-fluoro-2-naphthamide; N-[(1S,2R,4R)-7-azabicyclo[2.2.1]hept-2-yl]-5-cyano-2-naphthamide; N-[(1S,2R,4R)-7-azabicyclo[2.2.1]hept-2-yl]-5-chloro-2-naphthamide; N-[(1S,2R,4R)-7-azabicyclo[2.2.1]hept-2-yl]-5-bromo-2-naphthamide; N-[(1S,2R,4R)-7-azabicyclo[2.2.1]hept-2-yl]-5-iodo-2-naphthamide; N-[(1S,2R,4R)-7-azabicyclo[2.2.1]hept-2-yl]-5-nitro-2-naphthamide; N-[(1S,2R,4R)-7-azabicyclo[2.2.1]hept-2-yl]-5-ethynyl-2-naphthamide; N-[(1S,2R,4R)-7-azabicyclo[2.2.1]hept-2-yl]-5-trifluoromethyl-2-naphthamide; N-[(1S,2R,4R)-7-azabicyclo[2.2.1]hept-2-yl]-5-prop-1-ynyl-2-naphthamide; N-[(1S,2R,4R)-7-azabicyclo[2.2.1]hept-2-yl]-5-ethenyl-2-naphthamide; N-[(1S,2R,4R)-7-azabicyclo[2.2.1]hept-2-yl]-5-(3-hydroxyprop-1-ynyl)-2-naphthamide; 5-(acetylamino)-N-[(1S,2R,4R)-7-azabicyclo[2.2.1]hept-2-yl]-2-naphthamide; N-[(1S,2R,4R)-7-azabicyclo[2.2.1]hept-2-yl]-5-(formylamino)-2-naphthamide; N-[(1S,2R,4R)-7-azabicyclo[2.2.1]hept-2-yl]-8-methyl-2-naphthamide; N-[(1S,2R,4R) -7-azabicyclo[2.2.1]hept-2-yl]-8-hydroxy-2-naphthamide; N-[(1S,2R,4R)-7-azabicyclo[2.2.1]hept-2-yl]-8-methoxy-2-naphthamide; N-[(1S,2R,4R)-7-azabicyclo[2.2.1]hept-2-yl]-8-mercapto-2-naphthamide; N-[(1S,2R,4R)-7-azabicyclo[2.2.1]hept-2-yl]-8-methylthio-2-naphthamide; 8-Amino-N-[(1S,2R,4R) -7-azabicyclo[2.2.1]hept-2-yl]-2-naphthamide; N-[(1S,2R,4R)-7-azabicyclo[2.2.1]hept-2-yl]-8-methylamino-2-naphthamide; N-[(1S,2R,4R) -7-azabicyclo[2.2.1]hept-2-yl]-8-fluoro-2-naphthamide; N-[(1S,2R,4R) -7-azabicyclo[2.2.1]hept-2-yl]-8-cyano-2-naphthamide; N-[(1S,2R,4R)-7-azabicyclo[2.2.1]hept-2-yl]-8-chloro-2-naphthamide; N-[(1S,2R,4R)-7-azabicyclo[2.2.1]hept-2-yl]-8-bromo-2-naphthamide; N-[(1S,2R,4R)-7-azabicyclo[2.2.1]hept-2-yl]-8-iodo-2-naphthamide; N-[(1S,2R,4R)-7-azabicyclo[2.2.1]hept-2-yl]-8-nitro-2-naphthamide; N-[(1S,2R,4R)-7-azabicyclo[2.2.1]hept-2-yl]-8-ethynyl-2-naphthamide; N-[(1S,2R,4R)-7-azabicyclo[2.2.1]hept-2-yl]-8-trifluoromethyl-2-naphthamide; N-[(1S,2R,4R)-7-azabicyclo[2.2.1]hept-2-yl]-8-prop-1-ynyl-2-naphthamide; N-[(1S,2R,4R)-7-azabicyclo[2.2 .1]hept-2-yl]-8-ethenyl-2-naphthamide; N-[(1S,2R,4R)-7-azabicyclo[2.2.1]hept-2-yl]-8-(3-hydroxyprop-1-ynyl)-2-naphthamide; 8-(acetylamino)-N-[(1S,2R,4R)-7-azabicyclo[2.2.1]hept-2-yl]-2-naphthamide; N-[(1S,2R,4R)-7-azabicyclo[2.2.1]hept-2-yl]-8-(formylamino)-2-naphthamide; N-[(1S,2R,4R)-7-azabicyclo[2.2.1]hept-2-yl]-7-carbamoyl-2-naphthamide; N-[(1S,2R,4R) -7-azabicyclo[2.2.1]hept-2-yl]-5-carbamoyl-2-naphthamide; or N-[(1S,2R,4R) -7-azabicyclo[2.2.1]hept-2-yl]-8-carbamoyl-2-naphthamide.

The compound of Formula I or formula A-L-B, where the compound is as the free base, or a pharmaceutically acceptable salt thereof: N-((1S,2R,4R)-7-azabicyclo[2.2.1]hept-2-yl)indane-5-carboxamide.

Another group of compounds of Formula I includes compounds where each R_{Z} is independently H or R₃; and where each R₃ is independently alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —OR₈, —SR₈, —S(O)₂R₈, —S(O)R₈, —OS(O)₂R₈, F, Cl, Br, I, —NR₈R₈, —C(O)R₈, —C(S)R₈, —C(O)OR₈, —CN, —C(O)NR₈R₈, —NR₈C(O)R₈, —S(O)₂NR₈R₈, —NR₈S(O)₂R₈, —NO₂, —N(R₈)C(O)NR₈R₈, phenyl optionally substituted with 1-4 substituents independently selected from F, Cl, Br, I and R₁₅, or naphthyl optionally substituted with 1-4 substituents independently selected from F, Cl, Br, I, and R₁₅. Another group of compounds of Formula I includes compounds where two Rz groups are two R₃, where each R₃ is bound to the same carbon atom together to form —O or ═S.

Another group of compounds of Formula I includes compounds where Q is formula II having at least two substituents independently selected from the substituents as allowed herein and having at least one of those substituents being any one of the following: substituted alkyl, substituted alkenyl, substituted alkynyl, —OR₈, —SR₈, —S(O)₂R₈, —S(O)R₈, —OS(O)₂R₈, —N(R₈)₂, —C(O)R₈, —C(S)R₈, —C(O)OR₈, —C(O)N(R₈)₂, —NR₈C(O)R₈, —S(O)₂N(R₈)₂, —NR₈S(O)₂R₈, or —N(R₈)C(O)N(R₈)₂.

Another group of compounds of Formula I includes compounds wherein each R₃ is independently any one of the following: alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₈, —OR₈, —SR₈, —S(O)₂R₈, —S(O)R₈, —OS(O)₂R₈, F, Cl, Br, I, —N(R₈)₂, —C(O)R₈, —C(S)R₈, —C(O)OR₈, —CN, —C(O)N(R₈)₂, —NR₈C(O)R₈, —S(O)₂N(R₈)₂, —NR₈S(O)₂R₈, —NO₂, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅. Another group of compounds of Formula I includes compounds wherein two R₃ bound to the same carbon atom together to form ═O or ═S;

Another group of compounds of Formula I includes compounds wherein Q is substituted phenyl or substituted naphthyl having at least two substituents independently selected from R₃ and having at least one of those substituents being any one of the following: substituted alkyl, substituted alkenyl, substituted alkynyl, —OR₈, —SR₈, —S(O)₂R₈, —S(O)R₈, —OS(O)₂R₈, —N(R₈)₂, —C(O)R₈, —C(S)R₈, —C(O)OR₈, —C(O)N(R₈)₂, —NR₈C(O)R₈, —S(O)₂N(R₈)₂, —NR₈S(O)₂R₈, or —N(R₈)C(O)N(R₈)₂.

Another aspect of the invention includes a compound of formula A-L-B or a pharmaceutically acceptable salt thereof, wherein A is a 7-azabicyclo[2.2.1]heptane ring having 1S, 2R, and 4R stereochemistry; L is a linking moiety including an amide, a thioamide, an acrylamide, an acrylthioamide, a propiolamide, or a propiolthioamide, where the linking moiety is bonded to the C-2 carbon of the heptane ring in an exo orientation; and B is phenyl, naphthyl, or phenyl fused to a 5- or 6-membered saturated or partially unsaturated ring, all optionally substituted with up to 4 substituents where valency allows with any one or more of the following substituents as herein defined: alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —NO₂, —CN, F, Cl, Br, I, —OR₈, —SR₈, —S(O)₂R₈, —S(O)R₈, —OS(O)₂R₈, —N(R₈)₂, —C(O)R₈, —C(S)R₈, —C(O)OR₈, —C(O)N(R₈)₂, —NR₈C(O)R₈, —S(O)₂N(R₈)₂, —NR₈S(O)₂R₈, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally a carbon atom is substituted with ═O or ═S where valency allows. The B is bonded to L wherever valency allows on B.

The present invention also includes a pharmaceutical composition comprising a compound of Formula I or formula A-L-B or a pharmaceutically acceptable salt thereof and a pharmaceutically acceptable excipient. The pharmaceutical composition is administered rectally, topically, orally, sublingually, or parenterally for a therapeutically effective interval. The pharmaceutical composition is administered to deliver a compound of the present invention in an amount of from about 0.001 to about 100 mg/kg of body weight of said mammal per day. The pharmaceutical composition is also administered to deliver a compound of the present invention in an amount of from about 0.1 to about 50 mg/kg of body weight of said mammal per day.

A pharmaceutical composition comprising a compound of Formula I or formula A-L-B or a pharmaceutically acceptable salt thereof and an anti-psychotic agent. The pharmaceutical composition is administered to independently administer said compound and said agent rectally, topically, orally, sublingually, or parenterally for a therapeutically effective interval. The pharmaceutical composition is administered to deliver a compound of the present invention in an amount of from about 0.001 to about 100 mg/kg of body weight of said mammal per day. The pharmaceutical composition is also administered to deliver a compound of the present invention in an amount of from about 0.1 to about 50 mg/kg of body weight of said mammal per day.

The present invention also includes a use of a compound according to Formula I or formula A-L-B or pharmaceutically acceptable salt thereof for the preparation of a medicament for treating a disease or condition, wherein the mammal would receive symptomatic relief from the administration of a therapeutically effective amount of α7 nicotinic acetylcholine receptor agonist.

The present invention also includes a use of a compound according to Formula I or formula A-L-B or pharmaceutically acceptable salt thereof for the preparation of a medicament for treating a disease or condition, wherein the mammal would receive symptomatic relief from the administration of a therapeutically effective amount of α7 nicotinic acetylcholine receptor agonist, wherein the disease, or condition is any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

The present invention also includes a method for treating a disease or condition in a mammal in need thereof, wherein the mammal would receive symptomatic relief from the administration of an α7 nicotinic acetylcholine receptor agonist comprising administering to the mammal a therapeutically effective amount of a compound according to Formula I or formula A-L-B or pharmaceutically acceptable salt thereof.

The present invention also includes a method for treating a disease or condition in a mammal in need thereof comprising administering to the mammal a therapeutically effective amount of a compound according to Formula I or formula A-L-B or pharmaceutically acceptable salt thereof, wherein the disease or condition is any one or more or combination of the following: cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

The compounds of Formula I have optically active centers on the 7-azabicyclo[2.2.1]heptane ring which can exhibit a number of stereochemical configurations. The terms exo and endo are stereochemical prefixes that describe the relative configuration of a substituent on a bridge (not a bridgehead) of a bicyclic system. If a substituent is oriented toward the larger of the other bridges, it is endo. If a substituent is oriented toward the smaller bridge it is exo. Depending on the substitution on the carbon atoms, the endo and exo orientations can give rise to different stereoisomers. For instance, when carbons 1 and 4 are substituted with hydrogen and carbon 2 is bonded to a nitrogen containing species, the endo orientation gives rise to the possibility of a pair of enantiomers: either the 1S, 2S, 4R isomer or its enantiomer, the 1R, 2R, 4S isomer. Likewise, the exo orientation gives rise to the possibility of another pair of stereoisomers which are diastereomeric and C-2 epimeric with respect to the endo isomers: either the 1R, 2S, 4S isomer or its enantiomer, the 1S, 2R, 4R isomer. The compounds of this invention exist in the exo orientation. For example, when R₂═F₄═H, the absolute stereochemistry is exo-(2R,) for the compounds in Formula I.

Stereoselective syntheses and/or subjecting the reaction product to appropriate purification steps produces substantially optically pure materials. Suitable stereoselective synthetic procedures for producing optically pure materials are well known in the art, as are procedures for purifying racemic mixtures into optically pure fractions.

The compounds of the present invention have the exo orientation at the C-2 carbon and S configuration at the C-1 carbon and the R configuration at the C-2 and the C-4 carbons of the 7-azabicyclo[2.2.1]heptane ring. Unexpectedly, the inventive compounds exhibit much higher activity relative to compounds lacking the 1S, 2R, 4R stereochemistry within the 7-azabicyclo[2.2.1]heptane ring system. For example, the ratio of activities for compounds having the 1S, 2R, 4R configuration compared to other stereochemical configuarations of the 7-azabicyclo[2.2.1]heptane ring system may be greater than about 100. Although it is desirable that the stereochemical purity be as high as possible, absolute purity is not required. For example, pharmaceutical compositions can include one or more compounds, each having an exo 2R configuration, or mixtures of compounds having exo 2R and other configurations. In mixtures of compounds, those species possessing stereochemical configurations other than exo 2R act as diluents and tend to lower the activity of the pharmaceutical composition. Typically, pharmaceutical compositions including mixtures of compounds possess a larger percentage of species having the exo 2R configuration relative to other configurations.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF THE INVENTION

Surprisingly, we have found that compounds of the Formula I:

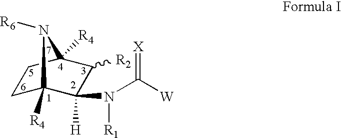

wherein the stereochemistry of the of the 7-azabicyclo[2.2.1]heptane ring is 1S, 4R and the nitrogen substituent at the C-2 carbon has the exo orientation and is R;

X is O or S;

W is -Q, —C═C-Q, or —C≡C-Q;

Q is aryl wherein the aryl can have a bond to the core molecule at any position where valency allows provided that there is only one said bond to the core molecule, or a group of formula II

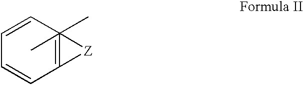

wherein the phenyl ring of formula II is optionally substituted with 1-4 substituents independently selected from F, Cl, Br, I, R₃, or a bond to the core molecule at any position where valency allows, provided that there is only one said bond to the core molecule;

Z is —C(R_{Z})₂—C(R_{Z})₂—C(R_{Z})₂—, —C(R_{Z})═C(R_{Z})—C(R_{Z})₂—, —C(R_{Z})₂—C(R_{Z})₂—C(R_{Z})₂—C(R_{Z})₂—, —C(R_{Z})═C(R_{Z})—C(R_{Z})₂—C(R_{Z})₂—, or —C(R_{Z})₂—C(R_{Z})═C(R_{Z})—C(R_{Z})₂—;

R_{Z} is H, R₃, or a bond to the core molecule at any position where valency allows, provided that there is only one said bond to the core molecule;

Aryl is phenyl, substituted phenyl, naphthyl, or substituted naphthyl;

R₁ is H, alkyl, cycloalkyl, halogenated alkyl, or aryl;

Alkyl is both straight- and branched-chain moieties having from 1-6 carbon atoms;

Halogenated alkyl is an alkyl moiety having from 1-6 carbon atoms and having 1 to (2n+1) substituent(s) independently selected from F, Cl, Br, or I where n is the maximum number of carbon atoms in the moiety;

Cycloalkyl is a cyclic alkyl moiety having from 3-6 carbon atoms;

Substituted phenyl is a phenyl having 1-4 substituents independently selected from R₃;

Substituted naphthyl is a naphthalene moiety having 1-4 substituents independently selected from R₃;

R₂ is H, alkyl, halogenated alkyl, substituted alkyl, cycloalkyl, or aryl;

Substituted alkyl is an alkyl moiety having from 1-6 carbon atoms and having 0-3 substituents independently selected from F, Cl, Br, or I and further having 1 substituent selected from —OR₁₀, —SR₁₀, —S(O)₂R₁₀, —S(O)R₁₀—OS(O)₂R₁₀, —N(R₁₀)₂, —C(O)R₁₀, —C(S)R₁₀, —C(O)OR₁₀, —C(O)N(R₁₀)₂, —CN, —NR₁₀C(O)R₁₀, —NR₁₀C(O)N(R₁₀)₂, —S(O)₂N(R₁₀)₂, —NR₁₀S(O)₂R₁₀, —NO₂, R₇, R₉, or phenyl optionally substituted with 1-4 substituents independently selected from F, Cl, Br, I, R₁₃, and R₁₅;

Each R₃ is independently alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —OR₈, —SR₈, —S(O)₂R₈, —S(O)R₈, —OS(O)₂R₈, F, Cl, Br, I, —N(R₈)₂, —C(O)R₈, —C(S)R₈, —C(O)OR₈, —CN, —C(O)N(R₈)₂, —NR₈C(O)R₈, —S(O)₂N(R₈)₂, —NR₈S(O)₂R₈, —NO₂, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally two R₃ groups bound to the same carbon atom together form ═O or ═S;

Alkenyl is straight- and branched-chain moieties having from 2-6 carbon atoms and having at least one carbon-carbon double bond;

Halogenated alkenyl is an unsaturated alkenyl moiety having from 2-6 carbon atoms and having 1 to (2n−1) substituent(s) independently selected from F, Cl, Br, or I where n is the maximum number of carbon atoms in the moiety;

Substituted alkenyl is an unsaturated alkenyl moiety having from 2-6 carbon atoms and having 0-3 substituents independently selected from F, Cl, Br, or I, and further having 1 substituent selected from R₇, R₉, —OR₁₀,—SR₁₀, —S(O)₂R₁₀, —S(O)R₁₀—OS(O)₂R₁₀, —N(R₁₀)₂, —C(O)R₁₀, —C(S)R₁₀, —C(O)OR₁₀, —C(O)N(R₁₀)₂, —CN, —NR₁₀C(O)R₁₀, —NR₁₀C(O)N(R₁₀)₂, —S(O)₂N(R₁₀)₂, —NR₁₀S(O)₂R₁₀, —NO₂, and phenyl optionally substituted with 1-4 substituents independently selected from F, Cl, Br, I, R₁₃, and R₁₅;

Alkynyl is straight- and branched-chained moieties having from 2-6 carbon atoms and having at least one carbon-carbon triple bond;

Halogenated alkynyl is an unsaturated alkynyl moiety having from 3-6 carbon atoms and having 1 to (2n−3) substituent(s) independently selected from F, Cl, Br, or I where n is the maximum number of carbon atoms in the moiety;

Substituted alkynyl is an unsaturated alkynyl moiety having from 3-6 carbon atoms and having 0-3 substituents independently selected from F, Cl, Br, or I, and further having 1 substituent selected from —R₇, —R₉, —OR₁₀, —SR₁₀, —S(O)₂R₁₀, —S(O)R₁₀—OS(O)₂R₁₀, —N(R₁₀)₂, —C(O)R₁₀, —C(S)R₁₀, —C(O)OR₁₀, —C(O)N(R₁₀)₂, —CN, —NR₁₀C(O)R₁₀, —NR₁₀C(O)N(R₁₀)₂, —S(O)₂N(R₁₀)₂, —NR₁₀S(O)₂R₁₀—NO₂, and phenyl optionally substituted with 1-4 substituents independently selected from F, Cl, Br, I, R₁₃, and R₁₅;

Halogenated cycloalkyl is a cyclic moiety having from 3-6 carbon atoms and having 1-4 substituents independently selected from F, Cl, Br, or I;

Substituted cycloalkyl is a cyclic moiety having from 3-6 carbon atoms and having 0-3 substituents independently selected from F, Cl, Br, or I, and further having 1 substituent selected from ═O, ═S, —R₇, —R₉, —OR₁₀, —SR₁₀, —S(O)₂R₁₀, —S(O)R₁₀—OS(O)₂R₁₀, —N(R₁₀)₂, —C(O)R₁₀, —C(S)R₁₀, —C(O)OR₁₀, —C(O)N(R₁₀)₂, —CN, —NR₁₀C(O)R₁₀, —NR₁₀C(O)N(R₁₀)₂, —S(O)₂N(R₁₀)₂, —NR₁₀S(O)₂R₁₀, —NO₂, and phenyl optionally substituted with 1-4 substituents independently selected from F, Cl, Br, I, R₁₃, and R₁₅;

Heterocycloalkyl is a cyclic moiety having 4-7 atoms with 1-2 atoms within the ring being —S—, —N(R₂₀)—, or —O—;

Halogenated heterocycloalkyl is a cyclic moiety having from 4-7 atoms with 1-2 atoms within the ring being —S—, —N(R₂₀)—, or —O—, and having 1-4 substituents independently selected from F, Cl, Br, or I;

Substituted heterocycloalkyl is a cyclic moiety having from 4-7 atoms with 1-2 atoms within the ring being —S—, —N(R₂₀)—, or —O— and having 0-3 substituents independently selected from F, Cl, Br, or I, and further having 1 substituent selected from ═O, ═S, —R₇, —R₉, —OR₁₀, —SR₁₀, —S(O)₂R₁₀, —S(O)R₁₀—OS(O)₂R₁₀, —N(R₁₀)₂, —C(O)R₁₀, —C(S)R₁₀, —C(O)OR₁₀, —C(O)N(R₁₀)₂, —CN, —NR₁₀C(O)R₁₀, —NR₁₀C(O)N(R₁₀)₂, —S(O)₂N(R₁₀)₂,—NR₁₀S(O)₂R₁₀, —NO₂, and phenyl optionally substituted with 1-4 substituents independently selected from F, Cl, Br, I, R₁₃, and R₁₅;

Lactam heterocycloalkyl is a cyclic moiety having from 4-7 atoms with one atom being only nitrogen with the bond to the lactam heterocycloalkyl thru said atom being only nitrogen and having a ═O on a carbon adjacent to said nitrogen, and having up to 1 additional ring atom being oxygen, sulfur, or nitrogen and further having 0-2 substituents selected from F, Cl, Br, I, or R₁₈ where valency allows;

Each R₄ is independently H, alkyl, or substituted alkyl;

R₆ is H, alkyl, an amino protecting group, or an alkyl group having 1-3 substituents selected from F, Cl, Br, I, —OH, —CN, —NH₂, —NH(alkyl), or —N(alkyl)₂;

R₇ is 5-membered heteroaromatic mono-cyclic moieties containing within the ring 1-3 heteroatoms independently selected from the group consisting of ═N—, —N(R₂₀)—, —O—, and —S—, and having 0-1 substituent selected from R₁₇ and further having 0-3 substituents independently selected from F, Cl, Br, or I, or R₇ is 9-membered fused-ring moieties having a 6-membered ring fused to a 5-membered ring including the formula

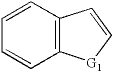

wherein G₁ is O, S or NR₂₀,

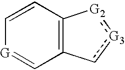

wherein G is C(R₁₄) or N, and each G₂ and G₃ are independently selected from C(R₁₄)₂, C(R₁₄), O, S, N, and N(R₂₀), provided that both G₂ and G₃ are not simultaneously O or S, or

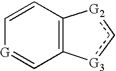

wherein G is C(R₁₄) or N, and each G₂ and G₃ are independently selected from C(R₁₄)₂, C(R₁₄), O, S, N, and N(R₂₀), each 9-membered bicyclic ring having 0-1 substituent selected from R₁₇ and 0-3 substituents independently selected from F, Cl, Br, or I, wherein the R₇ moiety attaches to other substituents as defined in formula I at any position on either ring as valency allows;

Each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅;

R₉ is 6-membered heteroaromatic mono-cyclic moieties containing within the ring 1-3 heteroatoms selected from ═N— and having 0-1 substituent selected from R₁₇ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₇ and 0-3 substituent(s) independently selected from F, Cl, Br, or I, wherein the R₉ moiety attaches to other substituents as defined in formula I at any position on either ring as valency allows;

Each R₁₀ is independently H, alkyl, cycloalkyl, heterocycloalkyl, R₇, R₉, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, or phenyl optionally substituted with 1-4 substituents independently selected from F, Cl, Br, I, R₁₃, and R₁₅;

Each R₁₁ is independently H, alkyl, cycloalkyl, heterocycloalkyl, halogenated alkyl, halogenated cycloalkyl, or halogenated heterocycloalkyl;

R₁₂ is alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —S(O)R₁₁, —S(O)₂R₁₁, —OS(O)₂R₁₁, —NR₁₁R₁₁, —C(O)R₁₁, —C(S)R₁₁, —C(O)OR₁₁, —NO₂, —CN, —C(O)N(R₁₁)₂, —NR₁₁C(O)R₁₁, —NR₁₁C(O)N(R₁₁)₂, —S(O)₂N(R₁₁)₂, or —NR₁₁S(O)₂R₁₁;

R₁₃ is —OR₁₁, —SR₁₁, —SOR₁₁, —SO₂R₁₁, —OSO₂R₁₁, —N(R₁₁)₂, —C(O)R₁₁, —C(O)OR₁₁, —C(S)R₁₁, —C(O)N(R₁₁)₂, —NO₂—CN, —CF₃, —NR₁₁C(O)R₁₁, —NR₁₁C(O)N(R₁₁)₂, —S(O)₂N(R₁₁)₂, or —NR₁₁S(O)₂R₁₁;

R₁₄ is H or R₁₉;

R₁₅ is lactam heterocycloalkyl, R₇, R₉, or R₁₉;

Each R₁₆ is independently H, alkyl, cycloalkyl, halogenated alkyl, or halogenated cycloalkyl;

R₁₇ is alkyl, cycloalkyl, or heterocycloalkyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₆C(O)R₁₆, —NR₁₆C(O)N(R₁₆)₂, —S(O)₂N(R₁₆)₂, and —NR₁₆S(O)₂R₁₆, and the cycloalkyl and heterocycloalkyl also being further optionally substituted with ═O or ═S;

R₁₈ is alkyl, substituted alkyl, halogenated alkyl, —OR₁₁, —CN, —NO₂, —N(R₁₀)₂;

R₁₉ is alkyl, cycloalkyl, heterocycloalkyl, phenyl, or naphthyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, or —NR₁₆S(O)₂R₁₆, and the cycloalkyl and heterocycloalkyl also being further optionally substituted with ═O or ═S;

R₂₀ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₂ and further having 0-3 substituents independently selected from F, Cl, Br, or I;

or pharmaceutical composition, pharmaceutically acceptable salt, racemic mixture, or pure enantiomer thereof useful to treat any one or more or combination of cognitive and attention deficit symptoms of Alzheimer's, neurodegeneration associated with diseases such as Alzheimer's disease, pre-senile dementia (mild cognitive impairment), senile dementia, schizophrenia, psychosis, attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

In another aspect, the invention includes methods of treating a mammal suffering from schizophrenia or psychosis by administering compounds of formula A-L-B or Formula I in conjunction with antipsychotic drugs. The compounds of formula A-L-B or Formula I and the antipsychotic drugs can be administered simultaneously or at separate intervals. When administered simultaneously the compounds of formula A-L-B or Formula I and the antipsychotic drugs can be incorporated into a single pharmaceutical composition. Alternatively, two separate compositions, i.e., one containing compounds of Formula I and the other containing antipsychotic drugs, can be administered simultaneously.

The present invention also includes the compounds of the present invention, pharmaceutical compositions containing the active compounds, and methods to treat the identified diseases.

Abbreviations which are well known to one of ordinary skill in the art may be used (e.g., “Ph” for phenyl, “Me” for methyl, “Et” for ethyl, “h” or “hr” for hour or hours, min for minute or minutes, and “rt” or “RT” for room temperature).

All temperatures are in degrees Centigrade.

Room temperature is within the range of 15-25 degrees Celsius.

AChR refers to acetylcholine receptor.

nAChR refers to nicotinic acetylcholine receptor.

Pre-senile dementia is also known as mild cognitive impairment.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to N,N-diisopropylethylamine.

MLA refers to methyllycaconitine.

Ether refers to diethyl ether.

HATU refers to O-(7-azabenzotriazol-1-yl)-N,N,N′,N′-tetramethyluronium hexafluorophosphate.

CDI refers to carbonyl diimidazole.

NMO refers to N-methylmorpholine-N-oxide.

TPAP refers to tetrapropylammonium perruthenate.

Halogen is F, Cl, Br, or I.

Amino protecting group includes, but is not limited to, carbobenzyloxy (CBz), 1,1 dimethylcarbamate, tert butoxy carbonyl (BOC) and the like. Examples of other suitable amino protecting groups are known to person skilled in the art and can be found in “Protective Groups in Organic synthesis,” 3rd Edition, authored by Theodora Greene and Peter Wuts.

Acrylamide or acrylthioamide is a moiety having the general structure —N(H)C(X)C═C—, where X is O or S, respectively, so formula A-L-B includes A-N(R₁)C(X)—C≡C—B.

Propiolamide or propiolthioamide is a moiety having the general structure —N(H)C(X)C≡C—, where X is O or S, respectively, so formula A-L-B includes A-N(R₁)C(X)—C≡C—B.

One of the most conventionally accepted ways of naming the compound pictured below is 3-amino-N-[(1S,2R,4R)-7-azabicyclo[2.2.1]hept-2-yl]-4-hydroxybenzamide, but for one ordinarily skilled in the art, the following name also describes the same compound, N-[(1S,2R,4R)-7-azabicyclo[2.2.1]hept-2-yl]-3-amino-4-hydroxybenzamide:

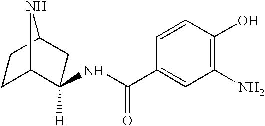

The two are used interchangeably in this patent.

Core molecule refers to the azabicyclo-moicty including the amide, thioamide, acrylamide, acrylthioamide, propiolamide; therefore, C═C or C≡C of W is within what is referred to as the core molecule. Hence, a bond to the core molecule would be the bond between the asterisk carbon of the C*(═X)—, C(═X)C═C*— or C(═X)C≡C*— and a carbon with sufficient valency of aryl, formula II, or B.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Lower alkyl is both straight- and branched-chain moieties having 1-4 carbon atoms.

Halogenated lower alkyl is lower alkyl having 1 to (2n+1) substituent(s) independently selected from F, Cl, Br, or I where n is the maximum number of carbon atoms in the moiety.

Substituted lower alkyl is lower alkyl having 0-3 substituents independently selected from F, Cl, Br, or I and further having 1 substituent selected from R₇, R₉, —CN, —NO₂, —OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀, or phenyl optionally substituted with 1-4 substituents independently selected from F, Cl, Br, I, R₁₃, and R₁₅.

Non-inclusive examples of heteroaryl compounds that fall within the definition of R₇ and R₉ include, but are not limited to, thienyl, benzothienyl, pyridyl, thiazolyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, benzoxazolyl, pyrazolyl, triazolyl, tetrazolyl, isoxazolyl, oxazolyl, pyrrolyl, isoquinolinyl, cinnolinyl, indazolyl, indolizinyl, phthalazinyl, pydridazinyl, triazinyl, isoindolyl, purinyl, oxadiazolyl, furazanyl, benzofurazanyl, benzothiophenyl, benzothiazolyl, quinazolinyl, quinoxalinyl, naphthridinyl, furopyridinyl, pyrrolopyridinyl, or thienopyridinyl. All isomeric forms of the non-inclusive named moieties are included, e.g., benzofuranyl includes 1-benzofuran-2-yl, 1-benzofuran-3-yl, 1-benzofuran-4-yl, 1-benzofuran-5-yl, 1-benzofuran-6-yl, 1-benzofuran-7-yl, 2-benzofuran-1-yl, 2-benzofuran-2-yl, 2-benzofuran-3-yl, 2-benzofuran-4-yl, or 2-benzofuran-5-yl. The non-inclusive examples of R₇ and R₉ may be substituted as allowed within the respective definition of R₇ and R₉ as valency allows. One of ordinary skill in the art can identify the allowed substitution by comparing the non-inclusive examples with the respective definitions of R₇ and R₉.

Non-inclusive examples of heterocycloalkyl include, but are not limited to, tetrahydrofurano, tetrahydropyrano, morpholino, pyrrolidino, piperidino, piperazine, azetidino, azetidinono, oxindolo, dihydroimidazolo, pyrrolidino, or isoxazolinyl.

Mammal denotes human and other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. M+H⁺ refers to the positive ion of a parent plus a hydrogen atom. M−H⁻ refers to the negative ion of a parent minus a hydrogen atom. M+Na⁺ refers to the positive ion of a parent plus a sodium atom. M+K⁺ refers to the positive ion of a parent plus a potassium atom. EI refers to electron impact. ESI refers to electrospray ionization. CI refers to chemical ionization. FAB refers to fast atom bombardment.

Compounds of the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired effect. As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

The amount of therapeutically effective compound(s) that is administered and the dosage regimen for treating a disease condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of compounds of the present invention. The pharmaceutical compositions may contain active ingredient in the range of about 0.001 to 100 mg/kg/day for an adult, preferably in the range of about 0.1 to 50 mg/kg/day for an adult. A total daily dose of about 1 to 1000 mg of active ingredient may be appropriate for an adult. The daily dose can be administered in one to four doses per day.

In addition to the compound(s) of the present invention, the composition for therapeutic use may also comprise one or more non-toxic, pharmaceutically acceptable carrier materials or excipients. The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, in the form of a pharmaceutical composition adapted to such a route, and in a dose effective for the treatment intended. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a large degree of sequence homology with α7 nAChR but functionally the two ligand-gated ion channels are very different. For example, α7 nAChR is rapidly inactivated, is highly permeable to calcium and is activated by acetylcholine and nicotine. On the other hand, 5HT₃R is inactivated slowly, is relatively impermeable to calcium and is activated by serotonin. These experiments suggest that the α7 nAChR and 5HT₃R proteins have some degree of homology, but function very differently. Indeed the pharmacology of the channels is very different. For example, Ondansetron, a highly selective 5HT₃R antagonist, has little activity at the α7 nAChR. The converse is also true. For example, GTS-21, a highly selective α7 nAChR agonist, has little activity at the 5HT₃R.

α7 nAChR is a ligand-gated Ca⁺⁺ channel formed by a homopentamer of α7 subunits. Previous studies have established that α-bungarotoxin (α-btx) binds selectively to this homopetameric, α7 nAChR subtype, and that α7 nAChR has a high affinity binding site for both α-btx and methyllycaconitine (MLA). α7 nAChR is expressed at high levels in the hippocampus, ventral tegmental area and ascending cholinergic projections from nucleus basilis to thalamocortical areas. α7 nAChR agonists increase neurotransmitter release, and increase cognition, arousal, attention, learning and memory.

Data from human and animal pharmacological studies establish that nicotinic cholinergic neuronal pathways control many important aspects of cognitive function including attention, learning and memory (Levin, E. D., Psychopharmacology, 108:417-31, 1992; Levin, E. D. and Simon B. B., Psychopharmacology, 138:217-30, 1998). For example, it is well known that nicotine increases cognition and attention in humans. ABT-418, a compound that activates α4β2 and α7 nAChR, improves cognition and attention in clinical trials of Alzheimer's disease and attention-deficit disorders (Potter, A. et. al., Psychopharmacology (Berl)., 142(4):334-42, March 1999; Wilens, T. E. et. al., Am. J. Psychiatry, 156(12):1931-7, December 1999). It is also clear that nicotine and selective but weak α7 nAChR agonists increase cognition and attention in rodents and non-human primates.

Schizophrenia is a complex multifactorial illness caused by genetic and non-genetic risk factors that produce a constellation of positive and negative symptoms. The positive symptoms include delusions and hallucinations and the negative symptoms include deficits in affect, attention, cognition and information processing. No single biological element has emerged as a dominant pathogenic factor in this disease. Indeed, it is likely that schizophrenia is a syndrome that is produced by the combination of many low penetrance risk factors. Pharmacological studies established that dopamine receptor antagonists are efficacious in treating the overt psychotic features (positive symptoms) of schizophrenia such as hallucinations and delusions. Clozapine, an “atypical” antipsychotic drug, is novel because it is effective in treating both the positive and some of the negative symptoms of this disease. Clozapine's utility as a drug is greatly limited because continued use leads to an increased risk of agranulocytosis and seizure. No other antipsychotic drug is effective in treating the negative symptoms of schizophrenia. This is significant because the restoration of cognitive functioning is the best predictor of a successfuil clinical and functional outcome of schizophrenic patients (Green, M. F., Am J Psychiatry, 153:321-30, 1996). By extension, it is clear that better drugs are needed to treat the cognitive disorders of schizophrenia in order to restore a better state of mental health to patients with this disorder.

One aspect of the cognitive deficit of schizophrenia can be measured by using the auditory event-related potential (P50) test of sensory gating. In this test, electroencepholographic (EEG) recordings of neuronal activity of the hippocampus are used to measure the subject's response to a series of auditory “clicks” (Adler, L. E. et. al., Biol. Psychiatry, 46:8-18, 1999). Normal individuals respond to the first click with greater degree than to the second click. In general, schizophrenics and schizotypal patients respond to both clicks nearly the same (Cullum, C. M. et. al., Schizophr. Res., 10:131-41, 1993). These data reflect a schizophrenic's inability to “filter” or ignore unimportant information. The sensory gating deficit appears to be one of the key pathological features of this disease (Cadenhead, K. S. et. al., Am. J Psychiatry, 157:55-9, 2000). Multiple studies show that nicotine normalizes the sensory deficit of schizophrenia (Adler, L. E. et. al., Am. J. Psychiatry, 150:1856-61, 1993). Pharmacological studies indicate that nicotine's effect on sensory gating is via the α7 nAChR (Adler, L. E. et. al., Schizophr. Bull., 24:189-202, 1998). Indeed, the biochemical data indicate that schizophrenics have 50% fewer of α7 nAChR receptors in the hippocampus, thus giving a rationale to partial loss of α7 nAChR functionality (Freedman, R. et. al., Biol. Psychiatry, 38:22-33, 1995). Interestingly, genetic data indicate that a polymorphism in the promoter region of the α7 nAChR gene is strongly associated with the sensory gating deficit in schizophrenia (Freedman, R. et. al., Proc. Nat'l Acad. Sci. USA, 94(2):587-92, 1997; Myles-Worsley, M. et. al., Am. J. Med. Genet, 88(5):544-50, 1999). To date, no mutation in the coding region of the α7 nAChR has been identified. Thus, schizophrenics express the same α7 nAChR as non-schizophrenics.

Selective α7 nAChR agonists may be found using a functional assay on FLIPR (see WO 00/73431 A2). FLIPR is designed to read the fluorescent signal from each well of a 96 or 384 well plate as fast as twice a second for up to 30 minutes. This assay may be used to accurately measure the functional pharmacology of α7 nACiR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

The compounds of the present invention are α7 nAChR agonists and may be used to treat a wide variety of diseases. For example, they may be used in treating schizophrenia, or psychosis.

Schizophrenia is a disease having multiple aspects. Currently available drugs are generally aimed at controlling the positive aspects of schizophrenia, such as delusions. One drug, Clozapine, is aimed at a broader spectrum of symptoms associated with schizophrenia. This drug has many side effects and is thus not suitable for many patients. Thus, there is a need for a drug to treat the cognitive and attention deficits associated with schizophrenia. Similarly, there is a need for a drug to treat the cognitive and attention deficits associated with schizoaffective disorders, or similar symptoms found in the relatives of schizophrenic patients.

Psychosis is a mental disorder characterized by gross impairment in the patient's perception of reality. The patient may suffer from delusions, and hallucinations, and may be incoherent in speech. His behavior may be agitated and is often incomprehensible to those around him. In the past, the term psychosis has been applied to many conditions that do not meet the stricter definition given above. For example, mood disorders were named as psychoses.

There are a variety of antipsychotic drugs. The conventional antipsychotic drugs include Chlorpromazine, Fluphenazine, Haloperidol, Loxapine, Mesoridazine, Molindone, Perphenazine, Pimozide, Thioridazine, Thiothixene, and Trifluoperazine. These drugs all have an affinity for the dopamine 2 receptor.

These conventional antipsychotic drugs have several side effects, including sedation, weight gain, tremors, elevated prolactin levels, akathisia (motor restlessness), dystonia and muscle stiffness. These drugs may also cause tardive dyskinesia. Unfortunately, only about 70% of patients with schizophrenia respond to conventional antipsychotic drugs. For these patients, atypical antipsychotic drugs are available.

Atypical antipsychotic drugs generally are able to alleviate positive symptoms of psychosis while also improving negative symptoms of the psychosis to a greater degree than conventional antipsychotics. These drugs may improve neurocognitive deficits. Extrapyramidal (motor) side effects are not as likely to occur with the atypical antipsychotic drugs, and thus, these atypical antipsychotic drugs have a lower risk of producing tardive dyskinesia. Finally these atypical antipsychotic drugs cause little or no elevation of prolactin. Unfortunately, these drugs are not free of side effects. Although these drugs each produce different side effects, as a group the side effects include: agranulocytosis; increased risk of seizures, weight gain, somnolence, dizziness, tachycardia, decreased ejaculatory volume, and mild prolongation of QTc interval.

In a combination therapy to treat multiple symptoms of diseases such as schizophrenia, the compounds of the present invention and the anti-psychotic drugs can be administered simultaneously or at separate intervals. When administered simultaneously the compounds of the present invention and the anti-psychotic drugs can be incorporated into a single pharmaceutical composition, e.g., a pharmaceutical combination therapy composition. Alternatively, two separate compositions, i.e., one containing compounds of the present invention and the other containing anti-psychotic drugs, can be administered simultaneously. Examples of anti-psychotic drugs, in addition to those listed above, include, but are not limited to, Thorazine, Mellaril, Trilafon, Navane, Stelazine, Permitil, Prolixin, Risperdal, Zyprexa, Seroquel, ZELDOX, Acetophenazine, Carphenazine, Chlorprothixene, Droperidol, Loxapine, Mesoridazine, Molindone, Ondansetron, Pimozide, Prochlorperazine, and Promazine.

A pharmaceutical combination therapy composition can include therapeutically effective amounts of the compounds of the present invention, noted above, and a therapeutically effective amount of anti-psychotic drugs. These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and maybe formulated as sustained relief dosage forms and the like.

When separately administered, therapeutically effective amounts of compositions containing compounds of the present invention and anti-psychotic drugs are administered on a different schedule. One may be administered before the other as long as the time between the two administrations falls within a therapeutically effective interval. A therapeutically effective interval is a period of time beginning when one of either (a) the compounds of the present invention, or (b) the anti-psychotic drugs is administered to a human and ending at the limit of the beneficial effect in the treatment of schizophrenia or psychosis of the combination of (a) and (b). The methods of administration of the compounds of the present invention and the anti-psychotic drugs may vary. Thus, either agent or both agents may be administered rectally, topically, orally, sublingually, or parenterally.

As discussed, the compounds of the present invention are α7 nAChR agonists. Therefore, as another aspect of the present invention, the compounds of the present invention may be used to treat a variety of diseases including cognitive and attention deficit symptoms of Alzheimer's, neurodegeneration associated with diseases such as Alzheimer's disease, pre-senile dementia (also known as mild cognitive impairment), and senile dementia.

Alzheimer's disease has many aspects, including cognitive and attention deficits. Currently, these deficits are treated with cholinesterase inhibitors. These inhibitors slow the break down of acetylcholine, and thereby provide a general nonspecific increase in the activity of the cholinergic nervous system. Since the drugs are nonspecific, they have a wide variety of side effects. Thus, there is a need for a drug that stimulates a portion of the cholinergic pathways and thereby provides improvement in the cognitive and attention deficits associated with Alzheimer's disease without the side effects created by nonspecific stimulation of the cholinergic pathways.

Neurodegeneration is a common problem associated with diseases such as Alzheimer's disease. While the current drugs treat some of the symptoms of this disease, they do not control the underlying pathology of the disease. Accordingly, it would be desirable to provide a drug that can slow the progress of Alzheimer's disease.

Pre-senile dementia (mild cognitive impairment) concerns memory impairment rather than attention deficit problems and otherwise unimpaired cognitive functioning. Mild cognitive impairment is distinguished from senile dementia in that mild cognitive impairment involves a more persistent and troublesome problem of memory loss for the age of the patient. There currently is no medication specifically identified for treatment of mild cognitive impairment, due somewhat to the newness of identifying the disease. Therefore, there is a need for a drug to treat the memory problems associated with mild cognitive impairment.

Senile dementia is not a single disease state. However, the conditions classified under this name frequently include cognitive and attention deficits. Generally, these deficits are not treated. Accordingly, there is a need for a drug that provides improvement in the cognitive and attention deficits associated with senile dementia.

As discussed, the compounds of the present invention are α7 nAChR agonists. Therefore, yet other diseases to be treated with compounds of the present invention include treating the cognitive and attention deficits as well as the neurodegeneration associated with any one or more or combination of the following: attention deficit disorder, attention deficit hyperactivity disorder, depression, anxiety, general anxiety disorder, post traumatic stress disorder, mood and affective disorders, amyotrophic lateral sclerosis, borderline personality disorder, traumatic brain injury, behavioral and cognitive problems associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.

Attention deficit disorder is generally treated with methylphenidate, an amphetamine-like molecule that has some potential for abuse. Accordingly, it would be desirable to provide a drug that treats attention deficit disorder while having fewer side effects than the currently used drug.

Attention deficit hyperactivity disorder, otherwise known as ADHD, is a neurobehavioral disorder affecting 3-5% of all American children. ADHD concerns cognitive alone or both cognitive and behavioral actions by interfering with a person's ability to stay on a task and to exercise age-appropriate inhibition. Several types of ADHD exist: a predominantly inattentive subtype, a predominantly hyperactive-impulsive subtype, and a combined subtype. Treatment may include medications such as methylphenidate, dextroamphetamine, or pemoline, which act to decrease impulsivity and hyperactivity and to increase attention. No “cure” for ADHD currently exists. Children with the disorder seldom outgrow it; therefore, there is a need for appropriate medicaments.

Depression is a mood disorder of varying lengths of normally several months to more than two years and of varying degrees of feelings involving sadness, despair, and discouragement. The heterocyclic antidepressants (HCA's) are currently the largest class of antidepressants, but monoamine oxidase inhibitors (MAOI's) are used in particular types of depression. Common side effects from HCA's are sedation and weight gain. In elderly patients with organic brain disease, the side effects from HCA's can also include seizures and behavioral symptoms. The main side effects from using MAOI's occur from dietary and drug interactions. Therefore, agents with fewer side effects would be useful.

Anxiety disorders (disorders with prominent anxiety or phobic avoidance), represent an area of umet medical needs in the treatment of psychiatric illness. See Diagnostic & Statistical Manual of Mental Disorders, IV (1994), pp 393-394, for various disease forms of anxiety.

General anxiety disorder (GAD) occurs when a person worries about things such as family, health, or work when there is no reason to worry and is unable not to worry. About 3 to 4% of the U.S. population has GAD during the course of a year. GAD most often strikes people in childhood or adolescence, but can begin in adulthood, too. It affects women more often than men. Currently, treatment involves cognitive-behavioral therapy, relaxation techniques, and biofeedback to control muscle tension and medications such as benzodiazepines, imipramine, and buspirone. These drugs are effective but all have side-effect liabilities. Therefore, there is a need of a pharmaceutical agent to address the symptoms with fewer side effects.

Anxiety also includes post-traumatic stress disorder (PTSD), which is a form of anxiety triggered by memories of a traumatic event that directly affected the patient or that the patient may have witnessed. The disorder commonly affects survivors of traumatic events including sexual assault, physical assault, war, torture, natural disasters, an automobile accident, an airplane crash, a hostage situation, or a death camp. The affliction also can affect rescue workers at an airplane crash or a mass shooting, someone who witnessed a tragic accident or someone who has unexpectedly lost a loved one. Treatment for PTSD includes cognitive-behavioral therapy, group psychotherapy, and medications such as Clonazepam, Lorazepam and selective serotonin-reuptake inhibitors such as Fluoxetine, Sertraline, Paroxetine, Citalopram and Fluvoxamine. These medications help control anxiety as well as depression. Various forms of exposure therapy (such as systemic desensitization and imaginal flooding) have all been used with PTSD patients. Exposure treatment for PTSD involves repeated reliving of the trauma, under controlled conditions, with the aim of facilitating the processing of the trauma. Therefore, there is a need for better pharmaceutical agents to treat post traumatic stress disorder.

Mood and affective disorders fall within a large group of diseases, including monopolar depression and bi-polar mood disorder. These diseases are treated with three major classes of compounds. The first group is the heterocyclic antidepressant (HCA's). This group includes the well-known tricyclic antidepressants. The second group of compounds used to treat mood disorders is the monoamine oxidase inhibitors (MAOI's) that are used in particular types of diseases. The third drug is lithium. Common side effects from HCA's are sedation and weight gain. In elderly patients with organic brain disease, the side effects of HCA's can also include seizures and behavioral symptoms. The main side effects from using MAOI's occur from dietary and drug interactions. Benign side effects from the use of lithium include, but are not limited to, weight gain, nausea, diarrhea, polyuria, polydipsia, and tremor. Toxic side effects from lithium can include persistent headache, mental confusion, and may reach seizures and cardiac arrhythmias. Therefore, agents with less side effects or interactions with food or other medications would be useful.

Borderline personality disorder, although not as well known as bipolar disorder, is more common. People having borderline personality disorder suffer from a disorder of emotion regulation. Pharmaceutical agents are used to treat specific symptoms, such as depression or thinking distortions.

Acquired immune deficiency syndrome (AIDS) results from an infection with the human immunodeficiency virus (HIV). This virus attacks selected cells and impairs the proper function of the immune, nervous, and other systems. HIV infection can cause other problems such as, but not limited to, difficulties in thinking, otherwise known as AIDS dementia complex. Therefore, there is a need to drugs to relieve the confusion and mental decline of persons with AIDS.

Amyotrophic lateral sclerosis, also known as Lou Gehrig's disease, belongs to a class of disorders known as motor neuron diseases wherein specific nerve cells in the brain and spinal cord gradually degenerate to negatively affect the control of voluntary movement. Currently, there is no cure for amyotrophic lateral sclerosis although patients may receive treatment from some of their symptoms and although Riluzole has been shown to prolong the survival of patients. Therefore, there is a need for a pharmaceutical agent to treat this disease.

Traumatic brain injury occurs when the brain is damaged from a sudden physical assault on the head. Symptoms of the traumatic brain injury include confusion and other cognitive problems. Therefore, there is a need to address the symptoms of confusion and other cognitive problems.

Brain tumors are abnormal growths of tissue found inside of the skull. Symptoms of brain tumors include behavioral and cognitive problems. Surgery, radiation, and chemotherapy are used to treat the tumor, but other agents are necessary to address associated symptoms. Therefore, there is a need to address the symptoms of behavioral and cognitive problems.

Persons with Down's syndrome have in all or at least some of their cells an extra, critical portion of the number 21 chromosome. Adults who have Down's syndrome are known to be at risk for Alzheimer-type dementia. Currently, there is no proven treatment for Down's syndrome. Therefore, there is a need to address the dementia associated with Down's syndrome.

Genetically programmed degeneration of neurons in certain areas of the brain cause Huntington's disease. Early symptoms of Huntington's disease include mood swings, or trouble learning new things or remembering a fact. Most drugs used to treat the symptoms of Huntington's disease have side effects such as fatigue, restlessness, or hyperexcitability. Currently, there is no treatment to stop or reverse the progression of Huntington's disease. Therefore, there is a need of a pharmaceutical agent to address the symptoms with fewer side effects.

Dementia with Lewy Bodies is a neurodegenerative disorder involving abnormal structures known as Lewy bodies found in certain areas of the brain. Symptoms of dementia with Lewy bodies include, but are not limited to, fluctuating cognitive impairment with episodic delirium. Currently, treatment concerns addressing the parkinsonian and psychiatric symptoms. However, medicine to control tremors or loss of muscle movement may actually accentuate the underlying disease of dementia with Lewy bodies. Therefore, there is a need of a pharmaceutical agent to treat dementia with Lewy bodies.

Parkinson's disease is a neurological disorder characterized by tremor, hypokinesia, and muscular rigidity. Currently, there is no treatment to stop the progression of the disease. Therefore, there is a need of a pharmaceutical agent to address Parkinson's.

Tardive dyskinesia is associated with the use of conventional antipsychotic drugs. This disease is characterized by involuntary movements most often manifested by puckering of the lips and tongue and/or writhing of the arms or legs. The incidence of tardive dyskinesia is about 5% per year of drug exposure among patients taking conventional antipsychotic drugs. In about 2% of persons with the disease, tardive dyskinesia is severely disfiguring. Currently, there is no generalized treatment for tardive dyskinesia. Furthermore, the removal of the effect-causing drugs is not always an option due to underlying problems. Therefore, there is a need for a pharmaceutical agent to address the symptoms of taridive dyskinesia.

Pick's disease results from a slowly progressive deterioration of social skills and changes in personality with the resulting symptoms being impairment of intellect, memory, and language. Common symptoms include memory loss, lack of spontaneity, difficulty in thinking or concentrating, and speech disturbances. Currently, there is no specific treatment or cure for Pick's disease but some symptoms can be treated with cholinergic and serotonin-boosting antidepressants. In addition, antipsychotic medications may alleviate symptoms in FTD patients who are experiencing delusions or hallucinations. Therefore, there is a need for a pharmaceutical agent to treat the progressive deterioration of social skills and changes in personality and to address the symptoms with fewer side effects.

Dysregulation of food intake associated with eating disease, including bulemia nervosa and anorexia nervosa, involve neurophysiological pathways. Anorexia nervosa is hard to treat due to patients not entering or remaining in after entering programs. Currently, there is no effective treatment for persons suffering from severe anorexia nervosa. Cognitive behavioral therapy has helped patients suffering from bulemia nervosa; however, the response rate is only about 50% and current treatment does not adequately address emotional regulation. Therefore, there is a need for pharmaceutical agents to address neurophysiological problems underlying diseases of dysregulation of food intake.

Cigarette smoking has been recognized as a major public health problem for a long time. However, in spite of the public awareness of health hazard, the smoking habit remains extraordinarily persistent and difficult to break. There are many treatment methods available, and yet people continue to smoke. Administration of nicotine transdermally, or in a chewing gum base is common treatments. However, nicotine has a large number of actions in the body, and thus can have many side effects. It is clear that there is both a need and a demand of long standing for a convenient and relatively easy method for aiding smokers in reducing or eliminating cigarette consumption. A drug that could selectively stimulate only certain of the nicotinic receptors would be useful in smoke cessation programs.

Smoke cessation programs may involve oral dosing of the drug of choice. The drug may be in the form of tablets. However, it is preferred to administer the daily dose over the waking hours, by administration of a series of incremental doses during the day. The preferred method of such administration is a slowly dissolving lozenge, troche, or chewing gum, in which the drug is dispersed. Another drug in treating nicotine addiction is Zyban. This is not a nicotine replacement, as are the gum and patch. Rather, this works on other areas of the brain, and its effectiveness is to help control nicotine craving or thoughts about cigarette use in people trying to quit. Zyban is not very effective and effective drugs are needed to assist smokers in their desire to stop smoking. These drugs may be administered transdermally through the use of skin patches. In certain cases, the drugs may be administered by subcutaneous injection, especially if sustained release formulations are used.

Drug use and dependence is a complex phenomenon, which cannot be encapsulated within a single definition. Different drugs have different effects, and therefore different types of dependence. Drug dependence has two basic causes, that is, tolerance and physical dependence. Tolerance exists when the user must take progressively larger doses to produce the effect originally achieved with smaller doses. Physical dependence exists when the user has developed a state of physiologic adaptation to a drug, and there is a withdrawal (abstinence) syndrome when the drug is no longer taken. A withdrawal syndrome can occur either when the drug is discontinued or when an antagonist displaces the drug from its binding site on cell receptors, thereby counteracting its effect. Drug dependence does not always require physical dependence.

In addition drug dependence often involves psychological dependence, that is, a feeling of pleasure or satisfaction when taking the drug. These feelings lead the user to repeat the drug experience or to avoid the displeasure of being deprived of the drug. Drugs that produce strong physical dependence, such as nicotine, heroin and alcohol are often abused, and the pattern of dependence is difficult to break. Drugs that produce dependence act on the CNS and generally reduce anxiety and tension; produce elation, euphoria, or other pleasurable mood changes; provide the user feelings of increased mental and physical ability; or alter sensory perception in some pleasurable manner. Among the drugs that are commonly abused are ethyl alcohol, opioids, anxiolytics, hypnotics, cannabis (marijuana), cocaine, amphetamines, and hallucinogens. The current treatment for drug-addicted people often involves a combination of behavioral therapies and medications. Medications, such as methadone or LAAM (levo-alpha-acetyl-methadol), are effective in suppressing the withdrawal symptoms and drug craving associated with narcotic addiction, thus reducing illicit drug use and improving the chances of the individual remaining in treatment. The primary medically assisted withdrawal method for narcotic addiction is to switch the patient to a comparable drug that produces milder withdrawal symptoms, and then gradually taper off the substitute medication. The medication used most often is methadone, taken orally once a day. Patients are started on the lowest dose that prevents the more severe signs of withdrawal and then the dose is gradually reduced. Substitutes can be used also for withdrawal from sedatives. Patients can be switched to long-acting sedatives, such as diazepam or phenobarbital, which are then gradually reduced.

Gilles de la Tourette's Syndrome is an inherited neurological disorder. The disorder is characterized by uncontrollable vocal sounds called tics and involuntary movements. The symptoms generally manifest in an individual before the person is 18 years of age. The movement disorder may begin with simple tics that progress to multiple complex tics, including respiratory and vocal ones. Vocal tics may begin as grunting or barking noises and evolve into compulsive utterances. Coprolalia (involuntary scatologic utterances) occurs in 50% of patients, Severe tics and coprolalia may be physically and socially disabling. Tics tend to be more complex than myoclonus, but less flowing than choreic movements, from which they must be differentiated. The patient may voluntarily suppress them for seconds or minutes.

Currently simple tics are often treated with benzodiazepines. For simple and complex tics, Clonidine may be used. Long-term use of Clonidine does not cause tardive dyskinesia; its limiting adverse effect is hypotension. In more severe cases, antipsychotics, such as Haloperidol may be required, but side effects of dysphoria, parkinsonism, akathisia, and tardive dyskinesia may limit use of such antipsychotics. There is a need for safe and effective methods for treating this syndrome.

Age-related macular degeneration (AMD) is a common eye disease of the macula which is a tiny area in the retina that helps produce sharp, central vision required for “straight ahead” activities that include reading and driving. Persons with AMD lose their clear, central vision. AMD takes two forms: wet and dry. In dry AMD, there is a slow breakdown of light-sensing cells in the macula. There currently is no cure for dry AMD. In wet AMD, new, fragile blood vessels growing beneath the macula as dry AMD worsens and these vessels often leak blood and fluid to cause rapid damage to the macula quickly leading to the loss of central vision. Laser surgery can treat some cases of wet AMD. Therefore, there is a need of a pharmaceutical agent to address AMD.

Glaucoma is within a group of diseases occurs from an increase in intraocular pressure causing pathological changes in the optical disk and negatively affects the field of vision. Medicaments to treat glaucoma either decrease the amount of fluid entering the eye or increase drainage of fluids from the eye in order to decrease intraocular pressure. However, current drugs have drawbacks such as not working over time or causing side effects so the eye-care professional has to either prescribe other drugs or modify the prescription of the drug being used. There is a need for safe and effective methods for treating problems manifesting into glaucoma.

Ischemic periods in glaucoma cause release of excitotoxic amino acids and stimulate inducible form of nitric oxide synthase (iNOS) leading to neurodegencration. Alpha 7 nicotinic agonists may stimulate the release of inhibitory amino acids such as GABA which will dampen hyperexcitablity. Alpha 7 nicotinic agonists are also directly neuroprotective on neuronal cell bodies. Thus alpha 7 nicotinic agonists have the potential to be neuroprotective in glaucoma.

Persons afflicted with pain often have what is referred to as the “terrible triad” of suffering from the pain, resulting in sleeplessness and sadness, all of which are hard on the afflicted individual and that individual's family. Pain can manifest itself in various forms, including, but not limited to, headaches of all severity, back pain, neurogenic, and pain from other ailments such as arthritis and cancer from its existence or from therapy to irradicate it. Pain can be either chronic (persistent pain for months or years) or acute (short-lived, immediate pain to inform the person of possible injury and need of treatment). Persons suffering from pain respond differently to individual therapies with varying degrees of success. There is a need for safe and effective methods for treating pain.

Finally, the compounds of the present invention may be used in combination therapy with typical and atypical anti-psychotic drugs (also called an anti-psychotic agent). All compounds within the present invention are useful for and may also be used in combination with each other to prepare pharmaceutical compositions. Such combination therapy lowers the effective dose of the anti-psychotic drug and thereby reduces the side effects of the anti-psychotic drugs. Some typical anti-psychotic drugs that may be used in the practice of the invention include Haldol. Some atypical anti-psychotic drugs include Ziprasidone, Olanzapine, Resperidone, and Quetiapine.

Compounds of Formula I can be prepared as shown in Scheme 1. Starting materials can be prepared by procedures described below or by procedures that would be well known to one of ordinary skill in organic chemistry. The variables used in Scheme 1 are defined below or as in the claims. The key step in the preparation of this class of compounds is the coupling of tert-butyl (1S,2R,4R)-(+)-2-amino-7-azabicyclo[2.2.1]heptane-7-carboxylate (Example 1) with the requisite acid chloride (Lv=Cl), mixed anhydride (e.g., Lv=diphenyl phosphoryl, bis(2-oxo-3-oxazolidinyl)phosphinyl, or acyloxy of the general formula of O—C(O)—R_{LV}, where R_{LV} includes phenyl or t-butyl), ester (e.g., Lv=alkyl, aryl, or electron deficient aryl), or carboxylic acid (Lv=OH) in the presence of an activating agent. Suitable activating reagents are well known in the art, for examples see Kiso, Y., Yajima, H. “Peptides” pp. 39-91, San Diego, Calif., Academic Press, (1995), and include, but are not limited to, agents such as carbodiimides, phosphonium and uronium salts (such as uronium salt HATU).

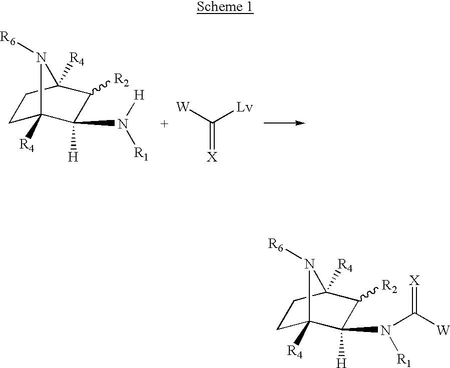

Preferably, tert-butyl (1S,2R,4R)-(+)-2-amino-7-azabicyclo[2.2.1]heptane-7-carboxylate can be coupled to the acid in the presence of an appropriate base, such as DIEA, and a uronium salt, such as HATU, in an aprotic medium, such as DMF, to give the desired amides. Alternatively, the acid is converted into a mixed anhydride by treatment with bis (2-oxo-3-oxazolidinyl) phosphinic chloride in the presence of TEA with CH₂Cl₂ or CHCl₃ as the solvent. The resulting anhydride solution is directly reacted with tert-butyl (1S,2R,4R)-(+)-2-amino-7-azabicyclo[2.2.1]heptane-7-carboxylate added neat or using CH₂Cl₂ or CHCl₃ as solvent. Furthermore, condensation of the amine with an ester (W—C(O)—O-alkyl or W—C(O)—O-(electron-deficient aryl)) in an alcoholic solvent such as ethanol at an elevated temperature will yield desired amides.

Treatment of the carboxamide with a sulfurating agent such as Lawesson's Reagent (2,4-bis(4-methoxyphenyl)-1,3-dithia-2,4-diphosphetane-2,4-disulfide) in, for instance, dioxane at an appropriate temperature provides the corresponding thioamide, e.g., X in formula I is S. See Lawesson et. al. in Bull. Soc. Chim. Belg., 229 (1978)), or P₄S₁₀ (see Chem. Rev., 45 (1961). Alternatively, one can react a dithiocarboxylic ester with the corresponding azbicyclo moiety to form the same thioamide.

There are various methods for the construction of the optionally substituted 7-azabicyclo[2.2.1]heptane ring system. For example, the independent work of Trudell (R₄=H, Zhang, C., Trudell, M. L., J. Org. Chem., 61, 7189-7191, 1996), and Schultz (R₄=Me, Schultz, A. G., Shen, M. S., Tetrahedron Lett., 22, 3347-3350, 1981) describes the utility of a Diels-Alder approach toward preparing this ring system with functionality suitable for further elaboration to the desired 2-amino-7-aza-bicyclo[2.2.1]heptane (Scheme 2). For instance, Trudell reports (Zhang, C., Trudell, M. L., Tetrahedron, 54, 8349-8354, 1998) that Diels-Alder adduct 1a (where R₆=methylcarbamate, R₄=H, and Lv=Br) could readily be functionalized at C-3 via reaction with organocopper species to introduce the substituent R₂ in 2a,b. Likewise, hydrogenolysis of adduct 1a,b or 2a,b followed by isomerization of the endo products as described by Singh (Singh, S., Basmadjian, G. P., Tetrahedron Lett., 38, 6829-6830, 1997) could provide access to the required exo acid 3a-d. Treatment of 3 with diphenylphosphoryl azide in the presence of a tertiary amine base (e.g., Et₃N) in a suitable solvent such as toluene, followed by warming of the intermediate acylazide in the presence of a suitable alcohol (e.g., benzyl alcohol) would effect the well-known Curtius rearrangement to provide a differentially protected bis carbamate which could be cleaved under typical hydrogenolysis conditions (e.g., 10% Pd/C, EtOH, H₂, ambient to 50 psi) to give the desired amine 4. Alternatively, the differentially protected bis carbamate might provide an attractive point of intervention for the chromatographic resolution of the individual 2-exo isomers prior to cleavage to amine 4.

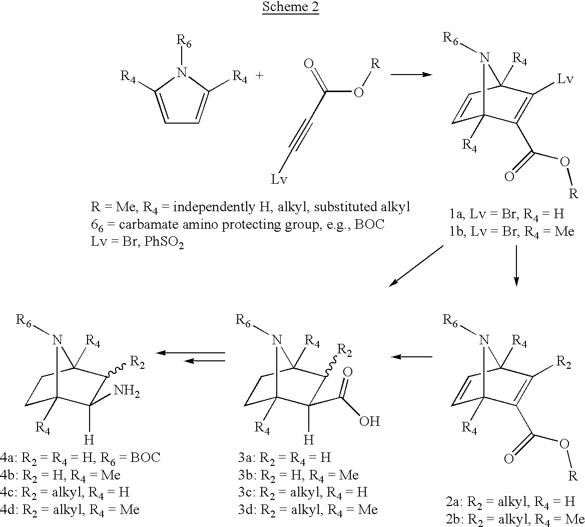

In the case where R₆=tert-butyloxycarbonyl, deprotection of the 7-aza group can be conveniently accomplished under acidic conditions in a suitable solvent such as methanol. After deprotection, the secondary amine may be functionalized with alkyl and substituted alkyl via reductive amination or alkylative procedures.

It will be apparent to those skilled in the art that the requisite carboxylic acids can be obtained through synthesis via literature procedures or through the slight modification thereof.

### Preparation of tert-butyl (1S,2R,4R)-2-amino-7-azabicyclo[2.2.1]heptane-7-carboxylate

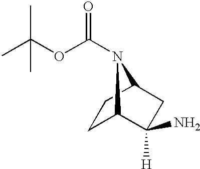

### Preparation of methyl-3-bromo-propiolate

Methyl propiolate (52 ml, 0.583 mol) is combined with recrystallized N-bromo-succinimide (120 g, 0.674 mol) in 1,700 ml acetone under nitrogen. The solution is treated with silver nitrate (9.9 g, 0.0583 mol) neat in a single lot and the reaction is stirred 6 h at RT. The acetone is removed under reduced pressure (25° C., bath temperature) to provide a gray slurry. The slurry is washed with 2×200 ml hexane, the gray solid is removed by filtration, and the filtrate is concentrated in vacuo to provide 95 g of a pale yellow oily residue. The crude material is distilled via short path under reduced pressure (65° C., about 25 mm Hg) into a dry ice/acetone cooled receiver to give 83.7 g (88%) of methyl-3-bromo-propiolate as a pale yellow oil. Anal. calc'd for C₄H₃BrO₂: C, 29.48; H, 1.86. Found: C, 29.09; H, 1.97.

### Preparation of 7-tert-butyl 2-methyl 3-bromo-7-azabicyclo[2.2.1]hepta-2,5-diene-2,7-dicarboxylate

Methyl-3-bromo-propiolate (83.7 g, 0.513 mol) is added to N-t-butyloxy-pyrrole (430 ml, 2.57 mol) under nitrogen. The dark mixture is warmed in a 90° C. bath for 30 h, is cooled, and the bulk of the excess N-t-butyloxy-pyrrole is removed in vacuo using a dry ice/acetone condenser. The dark oily residue is chromatographed over 1 kg silica gel (230-400 mesh) eluting with 0-15% EtOAc/hexane. The appropriate fractions are combined and concentrated to afford 97 g (57%) of 7-tert-butyl 2-methyl 3-bromo-7-azabicyclo[2.2.1]hepta-2,5-diene-2,7-dicarboxylate as a dark yellow oil. HRMS (FAB) caled for C₁₃H₁₆BrNO₄+H: 330.0341, found 330.0335 (M+H)⁺.

### Preparation of (+/−) endo-7-tert-butyl 2-methyl 7-azabicyclo[2.2.1]heptane -2,7-dicarboxylate

7-tert-Butyl 2-methyl 3-bromo-7-azabicyclo[2.2.1]hepta-2,5-diene-2,7-dicarboxylate (97 g, 0.294 mol) is added to 10% Pd/C (6.8 g) in 900 ml absolute EtOH in a PARR bottle. The suspension is diluted with a solution of NaHCO₃ (25 g, 0.301 mol) in 250 ml water and the mixture is hydrogenated at 50 PSI for 2.5 h. The catalyst is removed by filtration, is washed with fresh EtOH, and the filtrate is concentrated in vacuo to give a residue. The residue is partitioned between 1×200 ml saturated NaHCO₃ and CH₂Cl₂ (4×100 ml). The combined organic layer is dried over 1:1 anhydrous K₂CO₃/anhydrous MgSO₄ and concentrated in vacuo to afford 72.8 g (98%) of (+/−) endo-7-tert-butyl 2-methyl 7-azabicyclo[2.2.1]heptane -2,7-dicarboxylate. MS (EI) for C₁₄H₂₂O₄, m/z: 255 (M)⁺.

### Preparation of (+/−) exo-7-(tert-butoxycarbonyl)-7-azabicyclo[2.2.1]heptane -2-carboxylic acid

(+/−)Endo-7-tert-butyl 2-methyl 7-azabicyclo[2.2.1]heptane-2,7-dicarboxylate (72.8 g, 0.285 mol) is dissolved in 1000 ml dry MeOH in a dried flask under nitrogen. The solution is treated with solid NaOMe (38.5 g, 0.713 mol) neat, in a single lot and the reaction is warmed to reflux for 4 h. The mixture is cooled to 0° C., is treated with 400 ml water, and the reaction is stirred 1 h as it warms to RT. The mixture is concentrated in vacuo to about 400 ml and the pH of the aqueous residue is adjusted to 4.5 with 12N HCl. The precipitate is collected and dried. The tan, slightly tacky solid is washed with 2×100 ml 60% ether in hexane and is dried to provide 47 g (68%) of (+/−) exo-7-(tert-butoxycarbonyl)-7-azabicyclo[2.2.1]heptane-2-carboxylic acid as an off-white powder. HRMS (FAB) calc'd for C₁₂H₁₉NO₄+H: 242.1392, found 242.1390 (M+H)⁺.

### Preparation of (+/−) exo-tert-butyl 2-{[(benzyloxy)carbonyl]amino}-7-azabicyclo[2.2.1]heptane-7-carboxylate

(+/−)Exo-7-(tert-butoxycarbonyl)-7-azabicyclo[2.2.1]heptane-2-carboxylic acid (103.9 g, 0.430 mol) is combined with TEA (60 ml, 0.430 mol) in 1200 ml dry toluene in a dry flask under nitrogen. The solution is treated drop-wise with diphenylphosphoryl azide (92.8 ml, 0.430 mol), and is allowed to stir for 20 min at RT. The mixture is treated with benzyl alcohol (47.9 ml, 0.463 mol), and the reaction is stirred overnight at 55° C. The mixture is cooled, is extracted successively with 2×500 ml 5% citric acid, 2×500 ml water, 2×500 ml saturated sodium bicarbonate, and 500 ml saturated NaCl. The organic layer is dried over anhydrous MgSO₄ and concentrated in vacuo to an amber oil. The crude material is chromatographed over 900 g silica gel (230-400 mesh), eluting with 10-30% EtOAc/hexane. The appropriate fractions are combined and concentrated to give 106 g (71%) of (+/−) exo-tert-butyl 2-{[(benzyloxy)carbonyl]amino}-7-azabicyclo[2.2.1]heptane-7-carboxylate as a pale oil. ¹H NMR (CDCl₃) δ 1.29-1.60, 1.44, 1.62-2.01, 3.76-3.88, 4.10, 4.24, 5.10, 7.36 ppm.

### Preparation of (+/−) exo-tert-butyl 2-amino-7-azabicyclo[2.2.1]heptane-7-carboxylate

(+/−) Exo-tert-Butyl 2-{[(benzyloxy)carbonyl]amino}-7-azabicyclo[2.2.1]heptane-7-carboxylate (1.5 g, 4.33 mmol) is combined with 10% Pd/C (150 mg) in 40 ml EtOH in a 250 ml Parr shaker bottle. The mixture is hydrogenated at 50 PSI for 1.5 h. The catalyst is removed by filtration and the filtrate is concentrated in vacuo. The crude material is chromatographed over 30 g silica gel (230-400 mesh), eluting with 7% MeOH/CH₂Cl₂+1% conc. NH₄OH. The appropriate fractions are combined and concentrated to provide 606 mg (66%) of (+/−) exo-tert-butyl 2-amino-7-azabicyclo[2.2.1]heptane-7-carboxylate. HRMS (FAB) calcd for C₁₁H₂₀N₂O₂+H: 213.1603, found 213.1580 (M+H)⁺. This racemic mixture will be referenced as (+/−)-7-aza-[2.2.1]-Amine.

Resolution of racemic carboxylate mixture:

The isolated (+/−) exo-tert-butyl 2-{[(benzyloxy)carbonyl]amino}-7-azabicyclo[2.2.1]heptane-7-carboxylate is resolved via preparative chiral HPLC (50×500 mm Chiralcel OJ column, 30 deg. C., 70 mL/min. 10/90 (v/v) isopropanol/heptane). The resolution affords 40 g of tert-butyl (1S,2R,4R)-(+)-2{[(benzyloxy)carbonyl]amino}-7-azabicyclo[2.2.1]heptane-7-carboxylate and 42 g of tert-butyl-(1R,2S,4S)(−)-2{[(benzyloxy)carbonyl]amino}-7-azabicyclo[2.2.1]heptane-7-carboxylate.

The 2R enantiomer is triturated with 40 ml ether followed by 40 ml hexane (to remove lingering diastereo and enantiomeric impurities) and is dried to afford 30 g (56%) of purified tert-butyl (1S,2R,4R)-(+)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=22, (c 0.42, chloroform).

The 2S enantiomer is triturated with 40 ml ether followed by 40 ml hexane to give 35 g (66%) of purified tert-butyl (1R,2S,4S)-(−)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=−23, (c 0.39, chloroform).

Preparation of tert-butyl-(1S,2R,4R)-(+)-2-amino-7-azabicyclo[2.2.1]heptane-7-carboxylate ((2R)-7-aza-[2.2.1]-Amine).

tert-Butyl (1S,2R,4R)-(+)-2{[(benzyloxy)carbonyl]amino}-7-azabicyclo[2.2.1]heptane-7-carboxylate (9.5 g, 27.4 mmol) is combined with 950 mg 10% Pd/C in 75 ml absolute EtOH in a 500 ml Parr bottle. The reaction mixture is hydrogenated at 50 PSI for 3 h, the catalyst is removed by filtration, and the filter cake is washed with MeOH. The filtrate is concentrated in vacuo to give 6.4 g of a residue. The crude material is chromatographed over 200 g silica gel (230-400 mesh) eluting with 7% CH₃OH/CHCl₃ containing 1% conc. NH₄OH. The appropriate fractions are combined and concentrated to give 5.61 g (96%) of tert-butyl-(1S,2R,4R)-(+)-2-amino-7-azabicyclo[2.2.1 ]heptane-7-carboxylate as a pale oil. MS (EI) for C₁₁H₂₀N₂O₂, m/z: 212 (M)⁺. [α]²⁵_{D}=9, (c 0.67, CHCl₃). This compound will be referenced as (2R)-7-aza-[2.2.1]-Amine.

The following examples are provided as examples and are not intended to limit the scope of this invention to only those provided examples and named compounds. Also, the salts made in the examples are only exemplary and are not intended to limit the invention. Any pharmaceutically acceptable salt can be made by one of ordinary skill in the art. The invention includes the following examples in pure stereoisomeric form or as racemic mixtures.

### EXAMPLE 1

### 3-Amino-N-[(1S,2R,4R)-7-azabicyclo[2.2.1]hept-2-yl]-4-hydroxybenzamide dihydrochloride

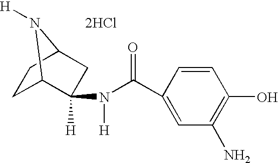

Coupling:

1,3-benzoxazole-5-carboxylic acid (179 mg, 1.1 mmol) is dissolved in CHCl₃ (5 ml) with TEA (0.15 ml, 1.1 mmol) and bis(2-oxo-3-oxazolidinyl)-phosphinic chloride (280 mg, 1.1 mmol) and stirred at rt for 0.5 h. (2R)-7-Aza-[2.2.1]-Amine (212 mg, 1.0 mmol) is dissolved in CHCl₃ (2 ml) and added drop-wise to the previous solution, stirring for 2 h at rt. The reaction is washed with saturated NaHCO₃ (1×10 ml), and the organic layer is dried over anhydrous K₂CO₃, filtered, and concentrated to an oil. The crude oil is chromatographed over 25 g slurry-packed silica, eluting with 40% EtOAc/hexane. The appropriate fractions are collected and concentrated to a yellow oil. The oil is dissolved in 1M HCl in MeOH (10 ml) and stirred overnight at rt. The volatiles are removed in vacuo and the residue is treated with IPA (2 ml), forming a precipitate. The slurry is filtered, and the cake is washed with ether, affording 151 mg (46%) of Example 1 as a white solid. MS for C₁₃H₁₇N₃O₂ 2HCl (EI) m/z: 247 (M)⁺.

### EXAMPLE 2

### N-[(1S,2R,4R)-7-Azabicyclo[2.2.1]hept-2-yl]-4-(4-fluorophenoxy)benzamide hydrochloride

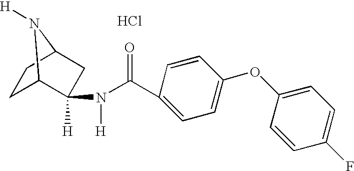

### Preparation of 4-(4-fluorophenoxy)benzoic acid

In a dry flask is placed 4-fluorobenzene boronic acid (2.11 g, 15.07 mmol, 2.00 equiv), methyl 4-hydroxybenzoate (1.15 g, 7.54 mmol, 1.00 equiv), copper(II) acetate (1.37 g, 7.54 mmol, 1.00 equiv), powdered molecular sieves (˜2g), TEA (5.24 ml, 37.68 mmol, 5.00 equiv) and then CH₂Cl₂ (75 ml). Dry air is bubbled through the reaction mixture for 18 h. The mixture is diluted with CH₂Cl₂, loaded onto silica gel and the product-and biphenyl by-product eluted with EtOAc-heptane (1:9, 1L) through a pad of silica gel. The desired fractions are collected, and the solvent is removed in vacuo to provide 1.9 g of the methyl ester that contained 92% desired methyl ester by NMR (1.69 g, 91%). This methyl ester (1.84 g, 7.5 mmol, 1.0 equiv) is stirred with dioxane (15 ml) until dissolved. LiOH (1.0N(aq), 15.0 ml, 2.0 equiv) is then added, and the reaction mixture is stirred for 18 h. HCl (1.0N, aqueous) is slowly added until pH<4. The resulting precipitate is collected by filtration, rinsed with water and dried at 60° C. in a vacuum oven for three days to provide 1.57g (90% from ester) of a white solid. MS for C₁₃H₉FO₃ (EI) m/z: 232 (M)⁺.

Example 2 is obtained using the coupling methods described for Example 1, making non-critical changes using 4-(4-fluorophenoxy)benzoic acid to obtain 87 mg (48%) of a white crystalline solid. MS for C₁₉H₁₉FN₂O₂, (ESI) m/z: 327 (M+H)⁺.

### EXAMPLE 3

### N-[(1S,2R,4R)-7-Azabicyclo[2.2.1]hept-2-yl]-2-naphthamide hydrochloride

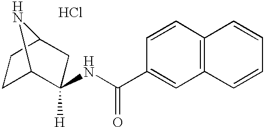

2-Naphthoic acid (129 mg, 0.75 mmol) is dissolved in DMF (5 ml) with DIEA (0.39 ml, 2.25 mmol) and (2R)-7-aza-[2.2.1]-Amine (175 mg, 0.83 mmol) and cooled to 0° C. HATU (285 mg, 0.75 mmol) is added portionwise and the reaction stirred overnight at rt allowing the ice bath to expire. Volatiles are removed in vacuo, and the crude material is chromatographed over 30 g slurry-packed silica, eluting with 35% EtOAc/hexane. The appropriate fractions are collected and concentrated. The residue is dissolved in 1M HCl in MeOH (5 ml) and stirred overnight. Slight heating is required at 50° C. for 1 h. Volatiles are again removed in vacuo, and the residue is treated with IPA (3 ml). The resulting precipitate is isolated via filtration, rinsed with ether, and dried to afford 129 mg (57%) of Example 3. HRMS (FAB) calcd for C₁₇H₁₈N₂O+H: 267.1497, found 267.1499 (M+H)⁺.

### EXAMPLE 4

### N-((1S,2R,4R)-7-Azabicyclo[2.2.1]hept-2-yl)indane-5-carboxamide hydrochloride

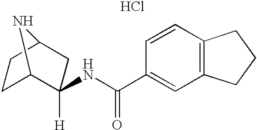

### Preparation of tert-Butyl (1S,2R,4R)-2-[(2,3-dihydro-1H-inden-5-ylcarbonyl)amino]-7-azabicyclo[2.2.1]heptane-7-carboxylate and tert-butyl (1R,2S,4S)-2-[(2,3-dihydro-1H-inden-5-ylcarbonyl)amino]-7-azabicyclo[2.2.1]heptane-7-carboxylate

Indane-5-carboxylic acid (Feiser and Hershberg, J. Med. Chem. Soc., 62, 49-51, 1940) (649 mg, 4.0 mmol) is combined with DIEA (1.29 mL, 8.0 mmol) and (+/−) 7-azabicyclo[2.2.1]heptan-2-amine (934 mg, 4.4 mmol) in DMF (20 mL), cooled to 0° C., is treated with HATU (1.52 g, 4.0 mmol) and is stirred for 4 h as the cooling bath expired. The mixture is concentrated to an amber oil (3.9 g) and chromatographed over 70 g slurry-packed silica gel, eluting with 25% EtOAc/hexane. The appropriate fractions are combined and concentrated to give a white foam (1.31 g). The material (1.3 g) is separated by preparative chiral HPLC utilizing a 5×50 cm Chiralpak AD column, 70 mL/min flow rate, 50% IPA/heptane mobile phase, 220 nm UV detection, 15 mL (650 mg) injections in IPA. Fraction A is collected from 14-19 min while Fraction B is collected from 21-29 min. The fractions are re-assayed as follows: 0.46×25 cm Chiralcel OD-H column, 0.5 mL/min. flow rate, 10% IPA/90% heptane mobile phase, 220 nm UW detection, 10 microliter injection. Fraction A elutes at 12.0 min (100% ee, 520 mg) while Fraction B elutes at 14.4 min (96.2% ee, 565 mg) under the assay conditions.

Fraction A is chromatographed over 20 g slurry-packed silica gel eluting with 30% EtOAc/hexane. The appropriate fractions are combined and concentrated to afford 479 mg (67%) of tert-butyl (1S,2R,4R)-2-[(2,3-dihydro-1H-inden-5-ylcarbonyl)amino]-7-azabicyclo[2.2.1]heptane-7-carboxylate as a white solid. MS (EI) m/z: 356 (M⁺).

Fraction B is chromatographed over 20 g slurry-packed silica gel eluting with 25% EtOAc/hexane. The appropriate fractions are combined and concentrated to afford 495 mg (69%) of tert-butyl (1R,2S,4S)-2-[(2,3-dihydro-1H-inden-5-ylcarbonyl)amino]-7-azabicyclo[2.2.1]heptane-7-carboxylate as a white solid. HRMS (FAB) calcd for C₂₁H₂₈N₂O₃+H: 357.2178, found 357.2184 (M+H)⁺.

tert-Butyl (1S,2R,4R)-2-[(2,3-dihydro-1H-inden-5-ylcarbonyl)amino]-7-azabicyclo[2.2.1]heptane-7-carboxylate (459 mg, 1.29 mmol) is dissolved in MeOH (20 mL), treated with 3 N methanolic HCl (4.5 mL) and stirred for 16 h at rt then heated to 50° C. for 7 h. The mixture is concentrated to dryness, dissolved in MeOH (0.5 mL), treated with IPA (1 mL) then diethyl ether (5 mL) until turbid. The solid is filtered under nitrogen and dried in a vacuum oven at 50° C. to afford 265 mg (70%) of Example 4. MS (EI) m/z: 256 (M⁺). [α]²⁵_{D}=−5, water (c=0.66).

### EXAMPLE 5

### N-[(1S,2R,4R)-7-Azabicyclo[2.2.1]hept-2-yl]-7-methoxy-2-naphthamide hydrochloride

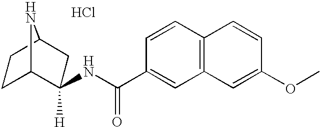

2-Cyano-7-methoxynaphthalene (501 mg, 2.74 mmol) (Kehr, Christiane, et al. Helv. Chim. Acta 1997, 80, 892-896; or Tschaen, D. M., et al. Synth. Commun. 1994, 24, 887-890) is suspended in 95% EtOH (5 mL). KOH (503 mg, 9.0 mmol) is added, and the resulting mixture is heated at reflux for 24 hours. The reaction is allowed to cool and then diluted with water (5 mL). Concentrated HCl is added until a pH of <2 is reached. The resulting precipitate is filtered, washed with water and dried at 70° C. under vacuum to yield 7-methoxy-2-naphthoic acid as a white solid (540 mg, 98%). ¹H NMR (300 MHz, DMSO-d6) δ 3.89, 7.29, 7.53, 7.81, 7.91, 7.92, 8.50, 13.01

7-Methoxy-2-naphthoic acid is coupled and with (2R)-7-aza-[2.2.1]-Amine and deprotected as described in Example 3 with non-critical changes to afford 247 mg (100%) of Example 5 as a white solid.

### Materials and Methods for Determining α7 nAChR Agonist Activity

Cell-based Assay for Measuring the EC₅₀ of α7 nAChR Agonists

Construction and Expression of the α7-5HT₃ Receptor:

The cDNA encoding the N-terminal 201 amino acids from the human α7 nAChR that contain the ligand binding domain of the ion channel was fused to the cDNA encoding the pore forming region of the mouse 5HT₃ receptor as described by Eisele J L, et al., Chimaeric nicotinic-serotonergic receptor combines distinct ligand binding and channel specificities, Nature (1993), December 2;366(6454):479-83, and modified by Groppi, et al., WO 00/73431. The chimeric α7-5HT₃ ion channel was inserted into pGS175 and pGS179 which contain the resistance genes for G-418 and hygromycin B, respectively. Both plasmids were simultaneously transfected into SH-EP1 cells and cell lines were selected that were resistant to both G-418 and hyrgromycin B. Cell lines expressing the chimeric ion channel were identified by their ability to bind fluorescent α-bungarotoxin on their cell surface. The cells with the highest amount of fluorescent α-bungarotoxin binding were isolated using a Fluorescent Activated Cell Sorter (FACS). Cell lines that stably expressed the chimeric α7-5HT₃ were identified by measuring fluorescent α-bungarotoxin binding after growing the cells in minimal essential medium containing nonessential amino acids supplemented with 10% fetal bovine serum, L-glutamine, 100 units/ml penicillin/streptomycin, 250 ng/mg fungizone, 400 μg/ml hygromycin B, and 400 μg/ml G-418 at 37° C. with 6% CO₂ in a standard mammalian cell incubator for at least 4 weeks in continuous culture.

Assay of the Activity of the Chimeric α7-5HT₃ Receptor

To assay the activity of the α7-5HT₃ ion channel, cells expressing the channel were plated into each well of either a 96 or 384 well dish (Corning #3614) and grown to confluence prior to assay. On the day of the assay, the cells were loaded with a 1:1 mixture of 2 mM Calcium Green 1, AM (Molecular Probes) dissolved in anhydrous DMSO and 20% pluronic F-127 (Molecular Probes). This solution was added directly to the growth media of each well to achieve a final concentration 2 μM. The cells were incubated with the dye for 60 min at 37° C. and then washed with a modified version of Earle's balanced salt solution (MMEBSS) as described in WO 00/73431. The ion conditions of the MMEBSS was adjusted to maximize the flux of calcium ion through the chimeric α7-5HT₃ ion channel as described in WO 00/73431. The activity of compounds on the chimeric α7-5HT₃ ion channel was analyzed on FLIPR. The instrument was set up with an excitation wavelength of 488 nanometers using 500 milliwatts of power. Fluorescent emission was measured above 525 nanometers with an appropriate F-stop to maintain a maximal signal to noise ratio. Agonist activity of each compound was measured by directly adding the compound to cells expressing the chimeric α7-5HT₃ ion channel and measuring the resulting increase in intracellular calcium that is caused by the agonist-induced activation of the chimeric ion channel. The assay is quantitative such that concentration-dependent increase in intracelluar calcium is measured as concentration-dependent change in Calcium Green fluorescence. The effective concentration needed for a compound to cause a 50% maximal increase in intracellular calcium is termed the EC₅₀. The examples of the present invention have EC₅₀ values from about 285 nM to about 32,600 nM.

Binding Constants:

Another way for measuring α7 nAChR agonist activity is to determine binding constants of a potential agonist in a competition binding assay. For α7 nAChR agonists, there is good correlation between functional EC₅₀ values using the chimeric α7-5HT₃ ion channel as a drug target and binding affinity of compounds to the endogenous α7 nAChR.

Membrane Preparation.

Male Sprague-Dawley rats (300-350 g) are sacrificed by decapitation and the brains (whole brain minus cerebellum) are dissected quickly, weighed and homogenized in 9 volumes/g wet weight of ice-cold 0.32 M sucrose using a rotating pestle on setting 50 (10 up and down strokes). The homogenate is centrifuged at 1,000×g for 10 minutes at 4° C. The supernatant is collected and centrifuged at 20,000×g for 20 minutes at 4° C. The resulting pellet is resuspended to a protein concentration of 1-8 mg/mL. Aliquots of 5 mL homogenate are frozen at −80° C. until needed for the assay. On the day of the assay, aliquots are thawed at rt and diluted with Kreb's −20 mM Hepes buffer pH 7.0 (at rt) containing 4.16 mM NaHCO₃, 0.44 mM KH₂PO₄, 127 mM NaCl, 5.36 mM KCl, 1.26 mM CaCl₂, and 0.98 mM MgCl₂, so that 25-150 μg protein are added per test tube. Proteins are determined by the Bradford method (Bradford, M. M., Anal. Biochem., 72, 248-254, 1976) using bovine serum albumin as the standard.

Binding Assay.

For saturation studies, 0.4 mL homogenate are added to test tubes containing buffer and various concentrations of radioligand, and are incubated in a final volume of 0.5 mL for 1 hour at 25° C. Nonspecific binding was determined in tissues incubated in parallel in the presence of 0.05 mls MLA for a final concentration of 1 μM, added before the radioligand. In competition studies, drugs are added in increasing concentrations to the test tubes before addition of 0.05 mls [³H]-MLA for a final concentration 3.0 to 4.0 nM. The incubations are terminated by rapid vacuum filtration through Whatman GF/B glass filter paper mounted on a 48 well Brandel cell harvester. Filters are pre-soaked in 50 mM Tris HCl pH 7.0-0.05% polyethylenimine. The filters are rapidly washed two times with 5 mL aliquots of cold 0.9% saline and then counted for radioactivity by liquid scintillation spectrometry.

Data Analysis.

In competition binding studies, the inhibition constant (Ki) was calculated from the concentration dependent inhibition of [³H]-MLA binding obtained from non-linear regression fitting program according to the Cheng-Prusoff equation (Cheng, Y. C. and Prussoff, W. H., Biochem. Pharmacol., 22, p. 3099-3108, 1973). Hill coefficients were obtained using non-linear regression (GraphPad Prism sigmoidal dose-response with variable slope).

What is claimed:

1. A compound of formula I: 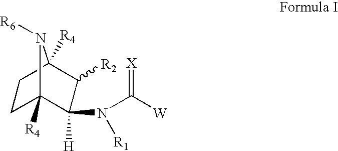
wherein the stereochemistry of the of the 7-azabicyclo[2.2.1]heptane ring is 1S, 4R and the nitrogen substituent at the C-2 carbon has the exo orientation and is R;
X is O or S;
W is -Q, —C═C-Q, or —C≡C-Q;
Q is aryl wherein the aryl can have a bond to the core molecule at any position where valency allows provided that there is only one said bond to the core molecule, or a group of formula II 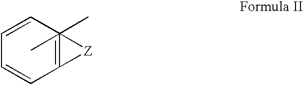
wherein the phenyl ring of formula II is optionally substituted with 1-4 substituents independently selected from F, Cl, Br, I, R₃, or a bond to the core molecule at any position where valency allows, provided that there is only one said bond to the core molecule;
Z is —C(R_{Z})₂—C(R_{Z})₂—C(R_{Z})₂—, —C(R_{Z})═C(R_{Z})—C(R_{Z})₂—, —C(R_{Z})₂—C(R_{Z})₂—C(R_{Z})₂—C(R_{Z})₂—, —C(R_{Z})═C(R_{Z})—C(R_{Z})₂—C(R_{Z})₂—, or —C(R_{Z})₂—C(R_{Z})═C(R_{Z})—C(R_{Z})₂—;
R_{Z} is H, R₃, or a bond to the core molecule at any position where valency allows, provided that there is only one said bond to the core molecule;
R₁ is H, alkyl, cycloalkyl, halogenated alkyl, or aryl;
R₂ is H, alkyl, halogenated alky, substituted alkyl, cycloalkyl, or aryl;
Each R₃ is independently alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alky, substituted alkenyl, substituted alkynyl, substituted cycloalkyl, substituted heterocycloalkyl, lactam heterocycloalkyl, R₇, R₉, —OR₈, —SR₈, —S(O)₂R₈, —S(O)R₈, —OS(O)₂R₈, F, Cl, Br, I, —N(R₈)₂, —C(O)R₈, —C(S)R₈, —C(O)OR₈, —CN, —C(O)N(R₈)₂, —NR₈C(O)R₈, —S(O)₂N(R₈)₂, —NR₈S(O)₂R₈, —NO₂, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally two R₃ groups bound to the same carbon atom together form ═O or ═S;
Each R₄ is independently H, alkyl, or substituted alkyl;
R₆ is H, alkyl, an amino protecting group, or an alkyl group having 1-3 substituents selected from F, Cl, Br, I, —OH, —CN, —NH₂, —NH(alkyl), or —N(alkyl)₂;
R₇ is 5-membered heteroaromatic mono-cyclic moieties containing within the ring 1-3 heteroatoms independently selected from the group consisting of ═N—, —N(R₂₀)—, —O—, and —S—, and having 0-1 substituent selected from R₁₇ and further having 0-3 substituents independently selected from F, Cl, Br, or I, or R₇ is 9-membered fused-ring moieties having a 6-membered ring fused to a 5-membered ring including the formula 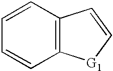
wherein G₁ is O, S or NR₂₀, 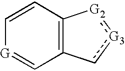
wherein G is C(R₁₄) or N, and each G₂ and G₃ are independently selected from C(R₁₄)₂, C(R₁₄), O, S, N, and N(R₂₀), provided that both G₂ and G₃ are not simultaneously O or S, or 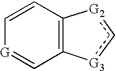
wherein G is C(R₁₄) or N, and each G₂ and G₃ are independently selected from C(R₁₄)₂, C(R₁₄), O, S, N, and N(R₂₀), each 9-membered bicyclic ring having 0-1 substituent selected from R₁₇ and 0-3 substituents independently selected from F, Cl, Br, or I, wherein the R₇ moiety attaches to other substituents as defined in formula I at any position on either ring as valency allows;
Each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅;
R₉ is 6-membered heteroaromatic mono-cyclic moieties containing within the ring 1-3 heteroatoms selected from ═N— and having 0-1 substituent selected from R₁₇ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₇ and 0-3 substituent(s) independently selected from F, Cl, Br, or I, wherein the R₉ moiety attaches to other substituents as defined in formula I at any position on either ring as valency allows;
Each R₁₀ is independently H, alkyl, cycloalkyl, heterocycloalkyl, R₇, R₉, alkyl substituted with 1 substituent selected from R₁₃, cycloalkyl substituted with 1 substituent selected from R₁₃, heterocycloalkyl substituted with 1 substituent selected from R₁₃, halogenated alkyl, halogenated cycloalkyl, halogenated heterocycloalkyl, or phenyl optionally substituted with 1-4 substituents independently selected from F, Cl, Br, I, R₁₃, and R₁₅;
Each R₁₁ is independently H, alkyl, cycloalkyl, heterocycloalkyl, halogenated alkyl, halogenated cycloalkyl, or halogenated heterocycloalkyl;
R₁₂ is alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —S(O)R₁₁, —S(O)₂R₁₁, —OS(O)₂R₁₁, —NR₁₁R₁₁, —C(O)R₁₁, —C(S)R₁₁, —C(O)OR₁₁, —NO₂, —CN, —C(O)N(R₁₁)₂, —NR₁₁C(O)R₁₁, —NR₁₁C(O)N(R₁₁)₂, —S(O)₂N(R₁₁)₂, or —NR₁₁S(O)₂R₁₁;
R₁₃ is —OR₁₁, —SR₁₁, —SOR₁₁, —SO₂R₁₁, —OSO₂R₁₁, —N(R₁₁)₂, —C(O)R₁₁, —C(O)OR₁₁, —C(S)R₁₁, —C(O)N(R₁₁)₂, —NO₂—CN, —CF₃, —NR₁₁C(O)R₁₁, —NR₁₁C(O)N(R₁₁)₂, —S(O)₂N(R₁₁)₂, or —NR₁₁S(O)₂R₁₁;
R₁₄ is H or R₁₉;
R₁₅ is lactam heterocycloalkyl, R₇, R₉, or R₁₉;
Each R₁₆ is independently H, alkyl, cycloalkyl, halogenated alkyl, or halogenated cycloalkyl;
R₁₇ is alkyl, cycloalkyl, or heterocycloalkyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, and —NR₁₆S(O)₂R₁₆, and the cycloalkyl and heterocycloalkyl also being further optionally substituted with ═O or ═S;
R₁₉ is alkyl, cycloalkyl, heterocycloalkyl, phenyl, or naphthyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, or —NR₁₆S(O)₂R₁₆, and the cycloalkyl and heterocycloalkyl also being further optionally substituted with ═O or ═S;
R₂₀ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₂ and further having 0-3 substituents independently selected from F, Cl, Br, or I;
or pharmaceutically acceptable salt thereof.

2. The compound of claim 1, wherein X is O.

3. The compound of claim 2, wherein R₁ is H, alkyl, or cycloalkyl, and wherein R₂ is H, alkyl, substituted alkyl, cycloalkyl, halogenated alkyl, or aryl.

4. The compound of claim 3, wherein Q is aryl.

5. The compound of claim 4, wherein each R₄ is independently H, lower alkyl, or substituted lower alkyl.

6. The compound according to claim 5, wherein R₆ is an amino protecting group.

7. The compound according to claim 5, wherein R₆ is H, or lower alkyl optionally substituted with up to 3 substituents independently selected from F, Cl, Br, I, —OH, —CN, —NH₂, —NH(alkyl), or —N(alkyl)₂.

8. The compound of claim 9, wherein R₁ is H or lower alkyl, and wherein R₂ is H or lower alkyl.

9. The compound of claim 8, wherein at least one R₄ is H and one R₄ is H or lower alkyl optionally substituted with 1 substituent selected from —CN, —NO₂, —OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀, or phenyl optionally substituted with up to 4 substitutents independently selected from F, Cl, Br, I, R₁₃, and R₁₅, provided that when said lower alkyl is optionally substituted, said lower alkyl can be further optionally substituted with up to 3 substituents independently selected from F, Cl, Br, and I, and further provided that R₁₀ is H, lower alkyl, or halogenated lower alkyl.

10. The compound according to claim 9, wherein R₁, R₂, and each R₄ are H.

11. The compound according to claim 10, wherein aryl is substituted phenyl.

12. The compound according to claim 11, wherein the compound is 3-amino-N-[(1S,2R,4R)-7-azabicyclo[2.2.1]hept-2-yl]-4-hydroxybenzamide; N-[(1S,2R,4R)-7-azabicyclo[2.2.1]hept-2-yl]-4-(4-fluorophenoxy)benzamide; or a pharmaceutically acceptable salt thereof.

13. The compound according to claim 11, wherein the compound is N-[(1S,2R,4R)-7-azabicyclo[2.2.1]hept-2-yl]-4-(4-hydroxyphenoxy)benzamide; N-[(1S,2R,4R)-7-azabicyclo[2.2.1]hept-2-yl]-4-(4-acetamidophenoxy)benzamide; N-[(1S,2R,4R)-7-azabicyclo[2.2.1]hept-2-yl]-4-phenoxybenzamide; N-[(1S,2R,4R)-7-azabicyclo[2.2.1]hept-2-yl]-4-benzylbenzamide; N-[(1S,2R,4R)-7-azabicyclo[2.2.1]hept-2-yl]-4-(phenylsulfanyl)benzamide; N-[(1S,2R,4R)-7-azabicyclo[2.2.1]hept-2-yl]-3-phenoxybenzamide; N-[(1S,2R,4R)-7-azabicyclo[2.2.1]hept-2-yl]-4-benzoylbenzamide; N-[(1S,2R,4R)-7-azabicyclo[2.2.1]hept-2-yl]-4-(2-fluorophenoxy)benzamide; N-[(1S,2R,4R)-7-azabicyclo[2.2.1]hept-2-yl]-4-(3-fluorophenoxy)benzamide; N-[(1S,2R,4R)-7-azabicyclo[2.2.1]hept-2-yl]-4-(2-chlorophenoxy)benzamide; N-[(1S,2R,4R)-7-azabicyclo[2.2.1]hept-2-yl]-4-(3-chlorophenoxy)benzamide; N-[(1S,2R,4R)-7-azabicyclo[2.2.1]hept-2-yl]-4-(4-chlorophenoxy)benzamide; N-[(1S,2R,4R)-7-azabicyclo[2.2.1]hept-2-yl]-4-(2-methoxyphenoxy)benzamide; N-[(1S,2R,4R)-7-azabicyclo[2.2.1]hept-2-yl]-4-(3-methoxyphenoxy)benzamide; N-[(1S,2R,4R)-7-azabicyclo[2.2.1]hept-2-yl]-4-(4-methoxyphenoxy)benzamide; N-[(1S,2R,4R)-7-azabicyclo[2.2.1]hept-2-yl]-4-(3-chlorophenylsulfanyl)benzamide; N-[(1S,2R,4R)-7-azabicyclo[2.2.1]hept-2-yl]-4-(4-chlorophenylsulfanyl)benzamide; N-[(1S,2R,4R)-7-azabicyclo[2.2.1]hept-2-yl]-4-(2-chlorophenylsulfanyl)benzamide; N-[(1S,2R,4R)-7-azabicyclo[2.2.1]hept-2-yl]-4-(4-methoxyphenylsulfanyl)-benzamide; N-[(1S,2R,4R)-7-azabicyclo[2.2.1]hept-2-yl]-4-(3-methoxyphenylsulfanyl)-benzamide; N-[(1S,2R,4R)-7-azabicyclo[2.2.1]hept-2-yl]-4-(2-methoxyphenylsulfanyl)-benzamide; N-[(1S,2R,4R)-7-azabicyclo[2.2.1]hept-2-yl]-4-phenoxybenzamide; N-[(1S,2R,4R)-7-azabicyclo[2.2.1]hept-2-yl]-4-(4-aminophenoxy)-benzamide; N-[(1S,2R,4R)-7-azabicyclo[2.2.1]hept-2-yl]-4-(3-aminophenoxy)-benzamide; N-[(1S,2R,4R)-7-azabicyclo[2.2.1]hept-2-yl]-4-(2-aminophenoxy)-benzamide; N-[(1S,2R,4R)-7-azabicyclo[2.2.1]hept-2-yl]-4-(4-methanesulfonylamino-phenoxy)-benzamide; N-[(1S,2R,4R)-7-azabicyclo[2.2.1]hept-2-yl]-4-(3-methanesulfonylamino-phenoxy)-benzamide; N-[(1S,2R,4R)-7-azabicyclo[2.2.1]hept-2-yl]-4-(2-methanesulfonylamino-phenoxy)-benzamide; N-[(1S,2R,4R)-7-azabicyclo[2.2.1]hept-2-yl]-4-(4-acetoxyphenoxy)-benzamide; N-[(1S,2R,4R)-7-azabicyclo[2.2.1]hept-2-yl]-4-(3-acetoxyphenoxy)-benzamide; N-[(1S,2R,4R)-7-azabicyclo[2.2.1]hept-2-yl]-4-(2-acetoxyphenoxy)-benzamide; N-[(1S,2R,4R)-7-azabicyclo[2.2.1]hept-2-yl]-4-(4-acetylphenoxy)-benzamide; N-[(1S,2R,4R)-7-azabicyclo[2.2.1]hept-2-yl]-4-(3-acetylphenoxy)-benzamide; N-[(1S,2R,4R)-7-azabicyclo[2.2.1]hept-2-yl]-4-(2-acetylphenoxy)-benzamide; N-[(1S,2R,4R)-7-azabicyclo[2.2.1]hept-2-yl]-4-(4-carbamoylphenoxy)-benzamide; N-[(1S,2R,4R)-7-azabicyclo[2.2.1]hept-2-yl]-4-(3-carbamoylphenoxy)-benzamide; N-[(1S,2R,4R)-7-azabicyclo[2.2.1]hept-2-yl]-4-(2-carbamoylphenoxy)-benzamide; N-[(1S,2R,4R)-7-azabicyclo[2.2.1]hept-2-yl]-4-(4-cyanophenoxy)-benzamide; N-[(1S,2R,4R)-7-azabicyclo[2.2.1]hept-2-yl]-4-(3-cyanophenoxy)-benzamide; N-[(1S,2R,4R)-7-azabicyclo[2.2.1]hept-2-yl]-4-(2-cyanophenoxy)-benzamide; N-[(1S,2R,4R)-7-azabicyclo[2.2.1]hept-2-yl]-4-(4-sulfamoylphenoxy)-benzamide; N-[(1S,2R,4R)-7-azabicyclo[2.2.1]hept-2-yl]-4-(3-sulfamoylphenoxy)-benzamide; N-[(1S,2R,4R)-7-azabicyclo[2.2.1]hept-2-yl]-4-(2-sulfamoylphenoxy)-benzamide; N-[(1S,2R,4R)-7-azabicyclo[2.2.1]hept-2-yl]-4-(thiophen-2-yloxy)-benzamide; N-[(1S,2R,4R)-7-azabicyclo[2.2.1]hept-2-yl]-4-(5-methylthiophen-2-yloxy)-benzamide; N-[(1S,2R,4R)-7-azabicyclo[2.2.1]hept-2-yl]-4-(5-chlorothiophen-2-yloxy)-benzamide; N-[(1S,2R,4R)-7-azabicyclo[2.2.1]hept-2-yl]-4-(5-methoxythiophen-2-yloxy)-benzamide; N-[(1S,2R,4R)-7-azabicyclo[2.2.1]hept-2-yl]-4-(5-trifluoromethylthiophen-2-yloxy)-benzamide; N-[(1S,2R,4R)-7-azabicyclo[2.2.1]hept-2-yl]-4-(5-acetylthiophen-2-yloxy)-benzamide; N-[(1S,2R,4R)-7-azabicyclo[2.2.1]hept-2-yl]-4-(5-acetaminothiophen-2-yloxy)-benzamide; N-[(1S,2R,4R)-7-azabicyclo[2.2.1]hept-2-yl]-4-(5-cyanothiophen-2-yloxy)-benzamide; N-[(1S,2R,4R)-7-azabicyclo[2.2.1]hept-2-yl]-4-(5-morpholin-4-yl-thiophen-2-yloxy)-benzamide; N-[(1S,2R,4R)-7-azabicyclo[2.2.1]hept-2-yl]-4-(4-methylthiophen-2-yloxy)-benzamide; N-[(1S,2R,4R)-7-azabicyclo[2.2.1]hept-2-yl]-4-(4-chlorothiophen-2-yloxy)-benzamide; N-[(1S,2R,4R)-7-azabicyclo[2.2.1]hept-2-yl]-4-(4-methoxythiophen-2-yloxy)-benzamide; N-[(1S,2R,4R)-7-azabicyclo[2.2.1]hept-2-yl]-4-(4-trifluoromethylthiophen-2-yloxy)-benzamide; N-[(1S,2R,4R)-7-azabicyclo[2.2.1]hept-2-yl]-4-(4-acetylthiophen-2-yloxy)-benzamide; N-[(1S,2R,4R)-7-azabicyclo[2.2.1]hept-2-yl]-4-(4-acetaminothiophen-2-yloxy)-benzamide; N-[(1S,2R,4R)-7-azabicyclo[2.2.1]hept-2-yl]-4-(4-cyanothiophen-2-yloxy)-benzamide; N-[(1S,2R,4R)-7-azabicyclo[2.2.1]hept-2-yl]-4-(4-morpholin-4-yl-thiophen-2-yloxy)-benzamide; N-[(1S,2R,4R)-7-azabicyclo[2.2.1]hept-2-yl]-4-(fuiran-2-yloxy)-benzamide; N-[(1S,2R,4R)-7-azabicyclo[2.2.1]hept-2-yl]-4-(5-methylfuran-2-yloxy)-benzamide; N-[(1S,2R,4R)-7-azabicyclo[2.2.1]hept-2-yl]-4-(5-chlorofuran-2-yloxy)-benzamide; N-[(1S,2R,4R)-7-azabicyclo[2.2.1]hept-2-yl]-4-(5-methoxyfuran-2-yloxy)-benzamide; N-[(1S,2R,4R)-7-azabicyclo[2.2.1]hept-2-yl]-4-(5-trifluoromethylfuran-2-yloxy)-benzamide; N-[(1S,2R,4R)-7-azabicyclo[2.2.1]hept-2-yl]-4-(5-acetylfuran-2-yloxy)-benzamide; N-[(1S,2R,4R)-7-azabicyclo[2.2.1]hept-2-yl]-4-(5-acetaminofuran-2-yloxy)-benzamide; N-[(1S,2R,4R)-7-azabicyclo[2.2.1]hept-2-yl]-4-(5-cyanofuran-2-yloxy)-benzamide; N-[(1S,2R,4R)-7-azabicyclo[2.2.1]hept-2-yl]-4-(5-morpholin-4-yl-furan-2-yloxy)-benzamide; N-[(1S,2R,4R)-7-azabicyclo[2.2.1]hept-2-yl]-4-(4-methylfuran-2-yloxy)-benzamide; N-[(1S,2R,4R)-7-azabicyclo[2.2.1]hept-2-yl]-4-(4-chlorofuran-2-yloxy)-benzamide; N-[(1S,2R,4R)-7-azabicyclo[2.2.1]hept-2-yl]-4-(4-methoxyfuran-2-yloxy)-benzamide; N-[(1S,2R,4R)-7-azabicyclo[2.2.1]hept-2-yl]-4-(4-trifluoromethylfuran-2-yloxy)-benzamide; N-[(1S,2R,4R)-7-azabicyclo[2.2.1]hept-2-yl]-4-(4-acetylfuran-2-yloxy)-benzamide; N-[(1S,2R,4R)-7-azabicyclo[2.2.1]hept-2-yl]-4-(4-acetaminofuran-2-yloxy)-benzamide; N-[(1S,2R,4R)-7-azabicyclo[2.2.1]hept-2-yl]-4-(4-cyanofuran-2-yloxy)-benzamide; N-[(1S,2R,4R)-7-azabicyclo[2.2.1]hept-2-yl]-4-(4-morpholin-4-yl-furan-2-yloxy)-benzamide; N-[(1S,2R,4R)-7-azabicyclo[2.2.1]hept-2-yl]-4-(oxazol-2-yloxy)-benzamide; N-[(1S,2R,4R)-7-azabicyclo[2.2.1]hept-2-yl]-4-(5-methyloxazol-2-yloxy)-benzamide; N-[(1S,2R,4R)-7-azabicyclo[2.2.1]hept-2-yl]-4-(5-chlorooxazol-2-yloxy)-benzamide; N-[(1S,2R,4R)-7-azabicyclo[2.2.1]hept-2-yl]-4-(5-methoxyoxazol-2-yloxy)-benzamide; N-[(1S,2R,4R)-7-azabicyclo[2.2.1]hept-2-yl]-4-(5-trifluoromethyloxazol-2-yloxy)-benzamide; N-[(1S,2R,4R)-7-azabicyclo[2.2.1]hept-2-yl]-4-(5-acetyloxazol-2-yloxy)-benzamide; N-[(1S,2R,4R)-7-azabicyclo[2.2.1]hept-2-yl]-4-(5-acetaminooxazol-2-yloxy)-benzamide; N-[(1S,2R,4R)-7-azabicyclo[2.2.1]hept-2-yl]-4-(5-cyanooxazol-2-yloxy)-benzamide; N-[(1S,2R,4R)-7-azabicyclo[2.2.1]hept-2-yl]-4-(5-morpholin-4-yl-oxazol-2-yloxy)-benzamide; N-[(1S,2R,4R)-7-azabicyclo[2.2.1]hept-2-yl]-4-(oxazol-2-yloxy)-benzamide; N-[(1S,2R,4R)-7-azabicyclo[2.2.1]hept-2-yl]-4-(4-methyloxazol-2-yloxy)-benzamide; N-[(1S,2R,4R)-7-azabicyclo[2.2.1]hept-2-yl]-4-(4-chlorooxazol-2-yloxy)-benzamide; N-[(1S,2R,4R)-7-azabicyclo[2.2.1]hept-2-yl]-4-(4-methoxyoxazol-2-yloxy)-benzamide; N-[(1S,2R,4R)-7-azabicyclo[2.2.1]hept-2-yl]-4-(4-trifluoromethyloxazol-2-yloxy)-benzamide; N-[(1S,2R,4R)-7-azabicyclo[2.2.1]hept-2-yl]-4-(4-acetyloxazol-2-yloxy)-benzamide; N-[(1S,2R,4R)-7-azabicyclo[2.2.1]hept-2-yl]-4-(4-acetaminooxazol-2-yloxy)-benzamide; N-[(1S,2R,4R)-7-azabicyclo[2.2.1]hept-2-yl]-4-(4-cyanooxazol-2-yloxy)-benzamide; N-[(1S,2R,4R)-7-azabicyclo[2.2.1]hept-2-yl]-4-(4-morpholin-4-yl-oxazol-2-yloxy)-benzamide; N-[(1S,2R,4R)-7-azabicyclo[2.2.1]hept-2-yl]-4-(oxazol-5-yloxy)-benzamide; N-[(1S,2R,4R)-7-azabicyclo[2.2.1]hept-2-yl]-4-(2-methyloxazol-5-yloxy)-benzamide; N-[(1S,2R,4R)-7-azabicyclo[2.2.1]hept-2-yl]-4-(2-chlorooxazol-5-yloxy)-benzamide; N-[(1S,2R,4R)-7-azabicyclo[2.2.1]hept-2-yl]-4-(2-methoxyoxazol-5-yloxy)-benzamide; N-[(1S,2R,4R)-7-azabicyclo[2.2.1]hept-2-yl]-4-(2-trifluoromethyloxazol-5-yloxy)-benzamide; N-[(1S,2R,4R)-7-azabicyclo[2.2.1]hept-2-yl]-4-(2-acetyloxazol-5-yloxy)-benzamide; N-[(1S,2R,4R)-7-azabicyclo[2.2.1]hept-2-yl]-4-(2-acetaminooxazol-5-yloxy)-benzamide; N-[(1S,2R,4R)-7-azabicyclo[2.2.1]hept-2-yl]-4-(2-cyanooxazol-5-yloxy)-benzamide; N-[(1S,2R,4R)-7-azabicyclo[2.2.1]hept-2-yl]-4-(2-morpholin-4-yl-oxazol-5-yloxy)-benzamide; N-[(1S,2R,4R)-7-azabicyclo[2.2.1]hept-2-yl]-4-(thiazol-2-yloxy)-benzamide; N-[(1S,2R,4R)-7-azabicyclo[2.2.1]hept-2-yl]-4-(5-methylthiazol-2-yloxy)-benzamide; N-[(1S,2R,4R)-7-azabicyclo[2.2.1]hept-2-yl]-4-(5-chlorothiazol-2-yloxy)-benzamide; N-[(1S,2R,4R)-7-azabicyclo[2.2.1]hept-2-yl]-4-(5-methoxythiazol-2-yloxy)-benzamide; N-[(1S,2R,4R)-7-azabicyclo[2.2.1]hept-2-yl]-4-(5-trifluoromethylthiazol-2-yloxy)-benzamide; N-[(1S,2R,4R)-7-azabicyclo[2.2.1]hept-2-yl]-4-(5-acetylthiazol-2-yloxy)-benzamide; N-[(1S,2R,4R)-7-azabicyclo[2.2.1]hept-2-yl]-4-(5-acetaminothiazol-2-yloxy)-benzamide; N-[(1S,2R,4R)-7-azabicyclo[2.2.1]hept-2-yl]-4-(5-cyanothiazol-2-yloxy)-benzamide; N-[(1S,2R,4R)-7-azabicyclo[2.2.1]hept-2-yl]-4-(5-morpholin-4-yl-thiazol-2-yloxy)-benzamide; N-[(1S,2R,4R)-7-azabicyclo[2.2.1]hept-2-yl]-4-(thiazol-2-yloxy)-benzamide; N-[(1S,2R,4R)-7-azabicyclo[2.2.1]hept-2-yl]-4-(4-methylthiazol-2-yloxy)-benzamide; N-[(1S,2R,4R)-7-azabicyclo[2.2.1]hept-2-yl]-4-(4-chlorothiazol-2-yloxy)-benzamide; N-[(1S,2R,4R)-7-azabicyclo[2.2.1]hept-2-yl]-4-(4-methoxythiazol-2-yloxy)-benzamide; N-[(1S,2R,4R)-7-azabicyclo[2.2.1]hept-2-yl]-4-(4-trifluoromethylthiazol-2-yloxy)-benzamide; N-[(1S,2R,4R)-7-azabicyclo[2.2.1]hept-2-yl]-4-(4-acetylthiazol-2-yloxy)-benzamide; N-[(1S,2R,4R)-7-azabicyclo[2.2.1]hept-2-yl]-4-(4-acetaminothiazol-2-yloxy)-benzamide; N-[(1S,2R,4R)-7-azabicyclo[2.2.1]hept-2-yl]-4-(4-cyanothiazol-2-yloxy)-benzamide; N-[(1S,2R,4R)-7-azabicyclo[2.2.1]hept-2-yl]-4-(4-morpholin-4-yl-thiazol-2-yloxy)-benzamide; N-[(1S,2R,4R)-7-azabicyclo[2.2.1]hept-2-yl]-4-(thiazol-5-yloxy)-benzamide; N-[(1S,2R,4R)-7-azabicyclo[2.2.1]hept-2-yl]-4-(2-methylthiazol-5-yloxy)-benzamide; N-[(1S,2R,4R)-7-azabicyclo[2.2.1]hept-2-yl]-4-(2-chlorothiazol-5-yloxy)-benzamide; N-[(1S,2R,4R)-7-azabicyclo[2.2.1]hept-2-yl]-4-(2-methoxythiazol-5-yloxy)-benzamide; N-[(1S,2R,4R)-7-azabicyclo[2.2.1]hept-2-yl]-4-(2-trifluoromethylthiazol-5-yloxy)-benzamide; N-[(1S,2R,4R)-7-azabicyclo[2.2.1]hept-2-yl]-4-(2-acetylthiazol-5-yloxy)-benzamide; N-[(1S,2R,4R)-7-azabicyclo[2.2.1]hept-2-yl]-4-(2-acetaminothiazol-5-yloxy)-benzamide; N-[(1S,2R,4R)-7-azabicyclo[2.2.1]hept-2-yl]-4-(2-cyanothiazol-5-yloxy)-benzamide; N-[(1S,2R,4R)-7-azabicyclo[2.2.1]hept-2-yl]-4-(2-morpholin-4-yl-thiazol-5-yloxy)-benzamide; N-[(1S,2R,4R)-7-azabicyclo[2.2.1]hept-2-yl]-4-([1,3,4]oxadiazol-2-yloxy)-benzamide; N-[(1S,2R,4R)-7-azabicyclo[2.2.1]hept-2-yl]-4-(5-methyl[1,3,4]oxadiazol-2-yloxy)-benzamide; N-[(1S,2R,4R)-7-azabicyclo[2.2.1]hept-2-yl]-4-(5-chloro[1,3,4]oxadiazol-2-yloxy)-benzamide; N-[(1S,2R,4R)-7-azabicyclo[2.2.1]hept-2-yl]-4-(5-methoxy[1,3,4]oxadiazol-2-yloxy)-benzamide; N-[(1S,2R,4R)-7-azabicyclo[2.2.1]hept-2-yl]-4-(5-trifluoromethyl[1,3,4]oxadiazol-2-yloxy)-benzamide; N-[(1S,2R,4R)-7-azabicyclo[2.2.1]hept-2-yl]-4-(5-acetyl[1,3,4]oxadiazol-2-yloxy)-benzamide; N-[(1S,2R,4R)-7-azabicyclo[2.2.1]hept-2-yl]-4-(5-acetamino[1,3,4]oxadiazol-2-yloxy)-benzamide; N-[(1S,2R,4R)-7-azabicyclo[2.2.1]hept-2-yl]-4-(5-cyano[1,3,4]oxadiazol-2-yloxy)-benzamide; N-[(1S,2R,4R)-7-azabicyclo[2.2.1]hept-2-yl]-4-(5-morpholin-4-yl-[1,3,4]oxadiazol-2-yloxy)-benzamide; N-[(1S,2R,4R)-7-azabicyclo[2.2.1]hept-2-yl]-4-([1,3,4]thiadiazol-2-yloxy)-benzamide; N-[(1S,2R,4R)-7-azabicyclo[2.2.1]hept-2-yl]-4-(5-methyl[1,3,4]thiadiazol-2-yloxy)-benzamide; N-[(1S,2R,4R)-7-azabicyclo[2.2.1]hept-2-yl]-4-(5-chloro[1,3,4]thiadiazol-2-yloxy)-benzamide; N-[(1S,2R,4R)-7-azabicyclo[2.2.1]hept-2-yl]-4-(5-methoxy[1,3,4]thiadiazol-2-yloxy)-benzamide; N-[(1S,2R,4R)-7-azabicyclo[2.2.1]hept-2-yl]-4-(5-trifluoromethyl[1,3,4]thiadiazol-2-yloxy)-benzamide; N-[(1S,2R,4R)-7-azabicyclo[2.2.1]hept-2-yl]-4-(5-acetyl[1,3,4]thiadiazol-2-yloxy)-benzamide; N-[(1S,2R,4R)-7-azabicyclo[2.2.1]hept-2-yl]-4-(5-acetamino[1,3,4]thiadiazol-2-yloxy)-benzamide; N-[(1S,2R,4R)-7-azabicyclo[2.2.1]hept-2-yl]-4-(5-cyano[1,3,4]thiadiazol-2-yloxy)-benzamide; N-[(1S,2R,4R)-7-azabicyclo[2.2.1]hept-2-yl]-4-(5-morpholin-4-yl-[1,3,4]thiadiazol-2-yloxy)-benzamide; N-[(1S,2R,4R)-7-azabicyclo[2.2.1]hept-2-yl]-4-(4-aminophenylsulfanyl)-benzamide; N-[(1S,2R,4R)-7-azabicyclo[2.2.1]hept-2-yl]-4-(3-aminophenylsulfanyl)-benzamide; N-[(1S,2R,4R)-7-azabicyclo[2.2.1]hept-2-yl]-4-(2-aminophenylsulfanyl)-benzamide; N-[(1S,2R,4R)-7-azabicyclo[2.2.1]hept-2-yl]-4-(4-methanesulfonylamino-phenylsulfanyl)-benzamide; N-[(1S,2R,4R)-7-azabicyclo[2.2.1]hept-2-yl]-4-(3-methanesulfonylamino-phenylsulfanyl)-benzamide; N-[(1S,2R,4R)-7-azabicyclo[2.2.1]hept-2-yl]-4-(2-methanesulfonylamino-phenylsulfanyl)-benzamide; N-[(1S,2R,4R)-7-azabicyclo[2.2.1]hept-2-yl]-4-(4-acetoxyphenylsulfanyl)-benzamide; N-[(1S,2R,4R)-7-azabicyclo[2.2.1]hept-2-yl]-4-(3-acetoxyphenylsulfanyl)-benzamide; N-[(1S,2R,4R)-7-azabicyclo[2.2.1]hept-2-yl]-4-(2-acetoxyphenylsulfanyl)-benzamide; N-[(1S,2R,4R)-7-azabicyclo[2.2.1]hept-2-yl]-4-(4-acetylphenylsulfanyl)-benzamide; N-[(1S,2R,4R)-7-azabicyclo[2.2.1]hept-2-yl]-4-(3-acetylphenylsulfanyl)-benzamide; N-[(1S,2R,4R)-7-azabicyclo[2.2.1]hept-2-yl]-4-(2-acetylphenylsulfanyl)-benzamide; N-[(1S,2R,4R)-7-azabicyclo[2.2.1]hept-2-yl]-4-(4-carbamoylphenylsulfanyl)-benzamide; N-[(1S,2R,4R)-7-azabicyclo[2.2.1]hept-2-yl]-4-(3-carbamoylphenylsulfanyl)-benzamide; N-[(1S,2R,4R)-7-azabicyclo[2.2.1]hept-2-yl]-4-(2-carbamoylphenylsulfanyl)-benzamide; N-[(1S,2R,4R)-7-azabicyclo[2.2.1]hept-2-yl]-4-(4-cyanophenylsulfanyl)-benzamide; N-[(1S,2R,4R)-7-azabicyclo[2.2.1]hept-2-yl]-4-(3-cyanophenylsulfanyl)-benzamide; N-[(1S,2R,4R)-7-azabicyclo[2.2.1]hept-2-yl]-4-(2-cyanophenylsulfanyl)-benzamide; N-[(1S,2R,4R)-7-azabicyclo[2.2.1]hept-2-yl]-4-(4-sulfamoylphenylsulfanyl)-benzamide; N-[(1S,2R,4R)-7-azabicyclo[2.2.1]hept-2-yl]-4-(3-sulfamoylphenylsulfanyl)-benzamide; N-[(1S,2R,4R)-7-azabicyclo[2.2.1]hept-2-yl]-4-(2-sulfamoylphenylsulfanyl)-benzamide; N-[(1S,2R,4R)-7-azabicyclo[2.2.1]hept-2-yl]-4-(4-hydroxyphenylsulfanyl)-benzamide; N-[(1S,2R,4R)-7-azabicyclo[2.2.1]hept-2-yl]-4-(3-hydroxyphenylsulfanyl)-benzamide; N-[(1S,2R,4R)-7-azabicyclo[2.2.1]hept-2-yl]-4-(2-hydroxyphenylsulfanyl)-benzamide; N-[(1S,2R,4R)-7-azabicyclo[2.2.1]hept-2-yl]-4-(4-acetamidophenylsulfanyl)-benzamide; N-[(1S,2R,4R)-7-azabicyclo[2.2.1]hept-2-yl]-4-(3-acetamidophenylsulfanyl)-benzamide; N-[(1S,2R,4R)-7-azabicyclo[2.2.1]hept-2-yl]-4-(2-acetamidophenylsulfanyl)-benzamide; N-[(1S,2R,4R)-7-azabicyclo[2.2.1]hept-2-yl]-4-(thiophen-2-ylsulfanyl)-benzamide; N-[(1S,2R,4R)-7-azabicyclo[2.2.1]hept-2-yl]-4-(5-methylthiophen-2-ylsulfanyl)-benzamide; N-[(1S,2R,4R)-7-azabicyclo[2.2.1]hept-2-yl]-4-(5-chlorothiophen-2-ylsulfanyl)-benzamide; N-[(1S,2R,4R)-7-azabicyclo[2.2.1]hept-2-yl]-4-(5-methoxythiophen-2-ylsulfanyl)-benzamide; N-[(1S,2R,4R)-7-azabicyclo[2.2.1]hept-2-yl]-4-(5-trifluoromethylthiophen-2-ylsulfanyl)-benzamide; N-[(1S,2R,4R)-7-azabicyclo[2.2.1]hept-2-yl]-4-(5-acetylthiophen-2-ylsulfanyl)-benzamide; N-[(1S,2R,4R)-7-azabicyclo[2.2.1]hept-2-yl]-4-(5-acetaminothiophen-2-ylsulfanyl)-benzamide; N-[(1S,2R,4R)-7-azabicyclo[2.2.1]hept-2-yl]-4-(5-cyanothiophen-2-ylsulfanyl)-benzamide; N-[(1S,2R,4R)-7-azabicyclo[2.2.1]hept-2-yl]-4-(5-morpholin-4-yl-thiophen-2-ylsulfanyl)-benzamide; N-[(1S,2R,4R)-7-azabicyclo[2.2.1]hept-2-yl]-4-(4-methylthiophen-2-ylsulfanyl)-benzamide; N-[(1S,2R,4R)-7-azabicyclo[2.2.1]hept-2-yl]-4-(4-chlorothiophen-2-ylsulfanyl)-benzamide; N-[(1S,2R,4R)-7-azabicyclo[2.2.1]hept-2-yl]-4-(4-methoxythiophen-2-ylsulfanyl)-benzamide; N-[(1S,2R,4R)-7-azabicyclo[2.2.1]hept-2-yl]-4-(4-trifluoromethylthiophen-2-ylsulfanyl)-benzamide; N-[(1S,2R,4R)-7-azabicyclo[2.2.1]hept-2-yl]-4-(4-acetylthiophen-2-ylsulfanyl)-benzamide; N-[(1S,2R,4R)-7-azabicyclo[2.2.1]hept-2-yl]-4-(4-acetaminothiophen-2-ylsulfanyl)-benzamide; N-[(1S,2R,4R)-7-azabicyclo[2.2.1]hept-2-yl]-4-(4-cyanothiophen-2-ylsulfanyl)-benzamide; N-[(1S,2R,4R)-7-azabicyclo[2.2.1]hept-2-yl]-4-(4-morpholin-4-yl-thiophen-2-ylsulfanyl)-benzamide; N-[(1S,2R,4R)-7-azabicyclo[2.2.1]hept-2-yl]-4-(furan-2-ylsulfanyl)-benzamide; N-[(1S,2R,4R)-7-azabicyclo[2.2.1]hept-2-yl]-4-(5-methylfuran-2-ylsulfanyl)-benzamide; N-[(1S,2R,4R)-7-azabicyclo[2.2.1]hept-2-yl]-4-(5-chlorofuran-2-ylsulfanyl)-benzamide; N-[(1S,2R,4R)-7-azabicyclo[2.2.1]hept-2-yl]-4-(5-methoxyfuran-2-ylsulfanyl)-benzamide; N-[(1S,2R,4R)-7-azabicyclo[2.2.1]hept-2-yl]-4-(5-trifluoromethylfuran-2-ylsulfanyl)-benzamide; N-[(1S,2R,4R)-7-azabicyclo[2.2.1]hept-2-yl]-4-(5-acetylfiran-2-ylsulfanyl)-benzamide; N-[(1S,2R,4R)-7-azabicyclo[2.2.1]hept-2-yl]-4-(5-acetaminofuran-2-ylsulfanyl)-benzamide; N-[(1S,2R,4R)-7-azabicyclo[2.2.1]hept-2-yl]-4-(5-cyanofuran-2-ylsulfanyl)-benzamide; N-[(1S,2R,4R)-7-azabicyclo[2.2.1]hept-2-yl]-4-(5-morpholin-4-yl-furan-2-ylsulfanyl)-benzamide; N-[(1S,2R,4R)-7-azabicyclo[2.2.1]hept-2-yl]-4-(4-methylfuran-2-ylsulfanyl)-benzamide; N-[(1S,2R,4R)-7-azabicyclo[2.2.1]hept-2-yl]-4-(4-chlorofuran -2-ylsulfanyl)-benzamide; N-[(1S,2R,4R)-7-azabicyclo[2.2.1]hept-2-yl]-4-(4-methoxyfuran-2-ylsulfanyl)-benzamide; N-[(1S,2R,4R)-7-azabicyclo[2.2.1]hept-2-yl]-4-(4-trifluoromethylfuran-2-ylsulfanyl)-benzamide; N-[(1S,2R,4R)-7-azabicyclo[2.2.1]hept-2-yl]-4-(4-acetylfuran -2-ylsulfanyl)-benzamide; N-[(1S,2R,4R)-7-azabicyclo[2.2.1]hept-2-yl]-4-(4-acetaminofuran -2-ylsulfanyl)-benzamide; N-[(1S,2R,4R)-7-azabicyclo[2.2.1]hept-2-yl]-4-(4-cyanofuran -2-ylsulfanyl)-benzamide; N-[(1S,2R,4R)-7-azabicyclo[2.2.1]hept-2-yl]-4-(4-morpholin -4-yl-furan-2-ylsulfanyl)-benzamide; N-[(1S,2R,4R)-7-azabicyclo[2.2.1]hept-2-yl]-4-(oxazol-2-ylsulfanyl)-benzamide; N-[(1S,2R,4R)-7-azabicyclo[2.2.1]hept-2-yl]-4-(5-methyloxazol-2-ylsulfanyl)-benzamide; N-[(1S,2R,4R)-7-azabicyclo[2.2.1]hept-2-yl]-4-(5-chlorooxazol-2-ylsulfanyl)-benzamide; N-[(1S,2R,4R)-7-azabicyclo[2.2.1]hept-2-yl]-4-(5-methoxyoxazol -2-ylsulfanyl)-benzamide; N-[(1S,2R,4R)-7-azabicyclo[2.2.1]hept-2-yl]-4-(5-trifluoromethyloxazol -2-ylsulfanyl)-benzamide; N-[(1S,2R,4R)-7-azabicyclo[2.2.1]hept-2-yl]-4-(5-acetyloxazol-2-ylsulfanyl)-benzamide; N-[(1S,2R,4R)-7-azabicyclo[2.2.1]hept-2-yl]-4-(5-acetaminooxazol-2-ylsulfanyl)-benzamide; N-[(1S,2R,4R)-7-azabicyclo[2.2.1]hept-2-yl]-4-(5-cyanooxazol -2-ylsulfanyl)-benzamide; N-[(1S,2R,4R)-7-azabicyclo[2.2.1]hept-2-yl]-4-(5-morpholin -4-yl-oxazol -2-ylsulfanyl)-benzamide; N-[(1S,2R,4R)-7-azabicyclo[2.2.1]hept-2-yl]-4-(oxazol -2-ylsulfanyl)-benzamide; N-[(1S,2R,4R)-7-azabicyclo[2.2.1]hept-2-yl]-4-(4-methyloxazol-2-ylsulfanyl)-benzamide; N-[(1S,2R,4R)-7-azabicyclo[2.2.1]hept -2-yl]-4-(4-chlorooxazol-2-ylsulfanyl)-benzamide; N-[(1S,2R,4R)-7-azabicyclo[2.2.1]hept-2-yl]-4-(4-methoxyoxazol-2-ylsulfanyl)-benzamide; N-[(1S,2R,4R)-7-azabicyclo[2.2.1]hept-2-yl]-4-(4-trifluoromethyloxazol -2-ylsulfanyl)-benzamide; N-[(1S,2R,4R)-7-azabicyclo[2.2.1]hept-2-yl]-4-(4-acetyloxazol -2-ylsulfanyl)-benzamide; N-[(1S,2R,4R)-7-azabicyclo[2.2.1]hept-2-yl]-4-(4-acetaminooxazol -2-ylsulfanyl)-benzamide; N-[(1S,2R,4R)-7-azabicyclo[2.2.1]hept-2-yl]-4-(4-cyanooxazol-2-ylsulfanyl)-benzamide; N-[(1S,2R,4R)-7-azabicyclo[2.2.1]hept-2-yl]-4-(4-morpholin-4-yl-oxazol-2-ylsulfanyl)-benzamide; N-[(1S,2R,4R)-7-azabicyclo[2.2.1]hept-2-yl]-4-(oxazol-5-ylsulfanyl)-benzamide; N-[(1S,2R,4R)-7-azabicyclo[2.2.1]hept-2-yl]-4-(2-methyloxazol -5-ylsulfanyl)-benzamide; N-[(1S,2R,4R)-7-azabicyclo[2.2.1]hept-2-yl]-4-(2-chlorooxazol -5-ylsulfanyl)-benzamide; N-[(1S,2R,4R)-7-azabicyclo[2.2.1]hept-2-yl]-4-(2-methoxyoxazol -5-ylsulfanyl)-benzamide; N-[(1S,2R,4R)-7-azabicyclo[2.2.1]hept-2-yl]-4-(2-trifluoromethyloxazol-5-ylsulfanyl)-benzamide; N-[(1S,2R,4R)-7-azabicyclo[2.2.1]hept-2-yl]-4-(2-acetyloxazol-5-ylsulfanyl)-benzamide; N-[(1S,2R,4R)-7-azabicyclo[2.2.1]hept-2-yl]-4-(2-acetaminooxazol-5-ylsulfanyl)-benzamide; N-[(1S,2R,4R)-7-azabicyclo[2.2.1]hept-2-yl]-4-(2-cyanooxazol -5-ylsulfanyl)-benzamide; N-[(1S,2R,4R)-7-azabicyclo[2.2.1]hept-2-yl]-4-(2-morpholin-4-yl-oxazol -5-ylsulfanyl)-benzamide; N-[(1S,2R,4R)-7-azabicyclo[2.2.1]hept-2-yl]-4-(thiazol -2-ylsulfanyl)-benzamide; N-[(1S,2R,4R)-7-azabicyclo[2.2.1]hept-2-yl]-4-(5-methylthiazol-2-ylsulfanyl)-benzamide; N-[(1S,2R,4R)-7-azabicyclo[2.2.1]hept-2-yl]-4-(5-chlorothiazol-2-ylsulfanyl)-benzamide; N-[(1S,2R,4R)-7-azabicyclo[2.2.1]hept-2-yl]-4-(5-methoxythiazol-2-ylsulfanyl)-benzamide; N-[(1S, 2R,4R)-7-azabicyclo[2.2.1]hept-2-yl]-4-(5-trifluoromethylthiazol -2-ylsulfanyl)-benzamide; N-[(1S,2R,4R)-7-azabicyclo[2.2.1]hept-2-yl]-4-(5-acetylthiazol -2-ylsulfanyl)-benzamide; N-[(1S,2R,4R)-7-azabicyclo[2.2.1]hept-2-yl]-4-(5-acetaminothiazol -2-ylsulfanyl)-benzamide; N-[(1S,2R,4R)-7-azabicyclo[2.2.1]hept -2-yl]-4-(5-cyanothiazol-2-ylsulfanyl)-benzamide; N-[(1S,2R,4R)-7-azabicyclo[2.2.1]hept-2-yl]-4-(5-morpholin-4-yl-thiazol-2-ylsulfanyl)-benzamide; N-[(1S,2R,4R)-7-azabicyclo[2.2.1]hept-2-yl]-4-(thiazol-2-ylsulfanyl)-benzamide; N-[(1S,2R,4R)-7-azabicyclo[2.2.1]hept-2-yl]-4-(4-methylthiazol -2-ylsulfanyl)-benzamide; N-[(1S,2R,4R)-7-azabicyclo[2.2.1]hept-2-yl]-4-(4-chlorothiazol -2-ylsulfanyl)-benzamide; N-[(1S,2R,4R)-7-azabicyclo[2.2.1]hept-2-yl]-4-(4-methoxythiazol -2-ylsulfanyl)-benzamide; N-[(1S,2R,4R)-7-azabicyclo[2.2.1]hept-2-yl]-4-(4-trifluoromethylthiazol-2-ylsulfanyl)-benzamide; N-[(1S,2R,4R)-7-azabicyclo[2.2.1]hept-2-yl]-4-(4-acetylthiazol-2-ylsulfanyl)-benzamide; N-[(1S,2R,4R)-7-azabicyclo[2.2.1]hept-2-yl]-4-(4-acetaminothiazol-2-ylsulfanyl)-benzamide; N-[(1S,2R,4R)-7-azabicyclo[2.2.1]hept-2-yl]-4-(4-cyanothiazol -2-ylsulfanyl)-benzamide; N-[(1S,2R,4R)-7-azabicyclo[2.2.1]hept-2-yl]-4-(4-morpholin-4-yl-thiazol -2-ylsulfanyl)-benzamide; N-[(1S,2R,4R)-7-azabicyclo[2.2.1]hept-2-yl]-4-(thiazol -5-ylsulfanyl)-benzamide; N-[(1S,2R,4R)-7-azabicyclo[2.2.1]hept-2-yl]-4-(2-methylthiazol-5-ylsulfanyl)-benzamide; N-[(1S,2R,4R)-7-azabicyclo[2.2.1]hept -2-yl]-4-(2-chlorothiazol-5-ylsulfanyl)-benzamide; N-[(1S,2R,4R)-7-azabicyclo[2.2.1]hept-2-yl]-4-(2-methoxythiazol-5-ylsulfanyl)-benzamide; N-[(1S,2R,4R)-7-azabicyclo[2.2.1]hept-2-yl]-4-(2-trifluoromethylthiazol -5-ylsulfanyl)-benzamide; N-[(1S,2R,4R)-7-azabicyclo[2.2.1]hept-2-yl]-4-(2-acetylthiazol -5-ylsulfanyl)-benzamide; N-[(1S,2R,4R)-7-azabicyclo[2.2.1]hept-2-yl]-4-(2-acetaminothiazol -5-ylsulfanyl)-benzamide; N-[(1S,2R,4R)-7-azabicyclo[2.2.1]hept-2-yl]-4-(2-cyanothiazol-5-ylsulfanyl)-benzamide; N-[(1S,2R,4R)-7-azabicyclo[2.2.1]hept-2-yl]-4-(2-morpholin-4-yl-thiazol-5-ylsulfanyl)-benzamide; N-[(1S,2R,4R)-7-azabicyclo[2.2.1]hept-2-yl]-4-([1 ,3,4]oxadiazol -2-ylsulfanyl)-benzamide; N-[(1S,2R,4R)-7-azabicyclo[2.2.1]hept-2-yl]-4-(5-methyl[1,3,4]oxadiazol-2-ylsulfanyl)-benzamide; N-[(1S,2R,4R)-7-azabicyclo[2.2.1]hept-2-yl]-4-(5-chloro[1,3,4]oxadiazol-2-ylsulfanyl)-benzamide; N-[(1S,2R,4R)-7-azabicyclo[2.2.1]hept-2-yl]-4-(5-methoxy[1,3,4]oxadiazol-2-ylsulfanyl)-benzamide; N-[(1S,2R,4R)-7-azabicyclo[2.2.1]hept-2-yl]-4-(5-trifluoromethyl[1,3,4]oxadiazol-2-ylsulfanyl)-benzamide; N-[(1S,2R,4R)-7-azabicyclo[2.2.1]hept-2-yl]-4-(5-acetyl[1,3,4]oxadiazol-2-ylsulfanyl)-benzamide; N-[(1S,2R,4R)-7-azabicyclo[2.2.1]hept-2-yl]-4-(5-acetamino[1,3,4]oxadiazol -2-ylsulfanyl)-benzamide; N-[(1S,2R,4R)-7-azabicyclo[2.2.1]hept-2-yl]-4-(5-cyano[1,3,4]oxadiazol-2-ylsulfanyl)-benzamide; N-[(1S,2R,4R)-7-azabicyclo[2.2.1]hept-2-yl]-4-(5-morpholin-4-yl-[1,3,4]oxadiazol-2-ylsulfanyl)-benzamide; N-[(1S,2R,4R)-7-azabicyclo[2.2.1]hept-2-yl]-4-([1,3,4]thiadiazol-2-ylsulfanyl)-benzamide; N-[(1S,2R,4R)-7-azabicyclo[2.2.1]hept-2-yl]-4-(5-methyl[1,3,4]thiadiazol-2-ylsulfanyl)-benzamide; N-[(1S,2R,4R)-7-azabicyclo[2.2.1]hept-2-yl]-4-(5-chloro[1,3,4]thiadiazol-2-ylsulfanyl)-benzamide; N-[(1S,2R,4R)-7-azabicyclo[2.2.1]hept-2-yl]-4-(5-methoxy[1,3,4]thiadiazol-2-ylsulfanyl)-benzamide; N-[(1S,2R,4R)-7-azabicyclo[2.2.1]hept-2-yl]-4-(5-trifluoromethyl[1,3,4]thiadiazol-2-ylsulfanyl)-benzamide; N-[(1S,2R,4R)-7-azabicyclo[2.2.1]hept-2-yl]-4-(5-acetyl[1,3,4]thiadiazol-2-ylsulfanyl)-benzamide; N-[(1S,2R,4R)-7-azabicyclo[2.2.1]hept-2-yl]-4-(5-acetamino[1,3,4]thiadiazol -2-ylsulfanyl)-benzamide; N-[(1S,2R,4R)-7-azabicyclo[2.2.1]hept-2-yl]-4-(5-cyano[1,3,4]thiadiazol-2-ylsulfanyl)-benzamide; N-[(1S,2R,4R)-7-azabicyclo[2.2.1]hept-2-yl]-4-(5-morpholin-4-yl-[1,3,4]thiadiazol-2-ylsulfanyl)-benzamide; N-[(1S,2R,4R)-7-azabicyclo[2.2.1]hept-2-yl]-4-(pyrrol-2-yloxy)-benzamide; N-[(1S,2R,4R)-7-azabicyclo[2.2.1]hept-2-yl]-4-(5-methylpyrrol -2-yloxy)-benzamide; N-[(1S,2R,4R)-7-azabicyclo[2.2.1]hept-2-yl]-4-(5-chloropyrrol -2-yloxy)-benzamide; N-[(1S,2R,4R)-7-azabicyclo[2.2.1]hept-2-yl]-4-(5-methoxypyrrol -2-yloxy)-benzamide; N-[(1S,2R,4R)-7-azabicyclo[2.2.1]hept-2-yl]-4-(5-trifluoromethylpyrrol-2-yloxy)-benzamide; N-[(1S,2R,4R)-7-azabicyclo[2.2.1]hept-2-yl]-4-(5-acetylpyrrol-2-yloxy)-benzamide; N-[(1S,2R,4R) -7-azabicyclo[2.2.1]hept-2-yl]-4-(5-acetaminopyrrol-2-yloxy)-benzamide; N-[(1S,2R,4R)-7-azabicyclo[2.2.1]hept-2-yl]-4-(5-cyanopyrrol-2-yloxy)-benzamide; N-[(1S, 2R, 4R)-7-azabicyclo[2.2.1]hept-2-yl]-4-(5-morpholin-4-yl-pyrrol-2-yloxy)-benzamide; N-[(1S,2R,4R)-7-azabicyclo[2.2.1]hept-2-yl]-4-(3H-imidazol-4-yloxy)-benzamide; N-[(1S,2R,4R)-7-azabicyclo[2.2.1]hept-2-yl]-4-(2-methyl-3H-imidazol -4-yloxy)-benzamide; N-[(1S,2R,4R)-7-azabicyclo[2.2.1]hept-2-yl]-4-(2-chloro-3H-imidazol -4-yloxy)-benzamide; N-[(1S,2R,4R)-7-azabicyclo[2.2.1]hept-2-yl]-4-(2-methoxy -3H-imidazol-4-yloxy)-benzamide; N-[(1S,2R,4R)-7-azabicyclo[2.2.1]hept-2-yl]-4-(2-trifluoromethyl-3H-imidazol-4-yloxy)-benzamide; N-[(1S,2R,4R)-7-azabicyclo[2.2.1]hept-2-yl]-4-(2-acetyl-3H-imidazol-4-yloxy)-benzamide; N-[(1S,2R,4R)-7-azabicyclo[2.2.1]hept-2-yl]-4-(2-acetamino-3H-imidazol -4-yloxy)-benzamide; N-[(1S,2R,4R)-7-azabicyclo[2.2.1]hept-2-yl]-4-(2-cyano-3H-imidazol -4-yloxy)-benzamide; N-[(1S,2R,4R)-7-azabicyclo[2.2.1]hept-2-yl]-4-(2-morpholin -4-yl-3H-imidazol-4-yloxy)-benzamide; N-[(1S,2R,4R)-7-azabicyclo[2.2.1]hept-2-yl]-4-(isoxazol-2-yloxy)-benzamide; N-[(1S,2R,4R)-7-azabicyclo[2.2.1]hept-2-yl]-4-(5-methylisoxazol-3-yloxy)-benzamide; N-[(1S,2R,4R)-7-azabicyclo[2.2.1]hept -2-yl]-4-(5-chloroisoxazol-3-yloxy)-benzamide; N-[(1S,2R,4R)-7-azabicyclo[2.2.1]hept-2-yl]-4-(5-methoxyisoxazol-3-yloxy)-benzamide; N-[(1S,2R,4R)-7-azabicyclo[2.2.1]hept-2-yl]-4-(5-trifluoromethylisoxazol-3-yloxy)-benzamide; N-[(1S,2R,4R)-7-azabicyclo[2.2.1]hept-2-yl]-4-(5-acetylisoxazol -3-yloxy)-benzamide; N-[(1S,2R,4R)-7-azabicyclo[2.2.1]hept-2-yl]-4-(5-acetaminoisoxazol -3-yloxy)-benzamide; N-[(1S,2R,4R)-7-azabicyclo[2.2.1]hept-2-yl]-4-(5-cyanoisoxazol -3-yloxy)-benzamide; N-[(1S,2R,4R)-7-azabicyclo[2.2.1]hept-2-yl]-4-(5-morpholin-4-yl-isoxazol-3-yloxy)-benzamide; N-[(1S,2R,4R)-7-azabicyclo[2.2.1]hept-2-yl]-4-(isothiazol-2-yloxy)-benzamide; N-[(1S,2R,4R)-7-azabicyclo[2.2.1]hept-2-yl]-4-(5-methylisothiazol-3-yloxy)-benzamide; N-[(1S,2R,4R)-7-azabicyclo[2.2.1]hept-2-yl]-4-(5-chloroisothiazol-3-yloxy)-benzamide; N-[(1S,2R,4R)-7-azabicyclo[2.2.1]hept-2-yl]-4-(5-methoxyisothiazol-3-yloxy)-benzamide; N-[(1S,2R,4R)-7-azabicyclo[2.2.1]hept-2-yl]-4-(5-trifluoromethylisothiazol -3-yloxy)-benzamide; N-[(1S,2R,4R)-7-azabicyclo[2.2.1]hept-2-yl]-4-(5-acetylisothiazol -3-yloxy)-benzamide; N-[(1S,2R,4R)-7-azabicyclo[2.2.1]hept-2-yl]-4-(5-acetaminoisothiazol-3-yloxy)-benzamide; N-[(1S,2R,4R)-7-azabicyclo[2.2.1]hept-2-yl]-4-(5-cyanoisothiazol-3-yloxy)-benzamide; N-[(1S,2R,4R)-7-azabicyclo[2.2.1]hept-2-yl]-4-(5-morpholin-4-yl-isothiazol -3-yloxy)-benzamide; N-[(1S,2R,4R)-7-azabicyclo[2.2.1]hept-2-yl]-4-(pyrrol-2-ylsulfanyl)-benzamide; N-[(1S,2R,4R)-7-azabicyclo[2.2.1]hept-2-yl]-4-(5-methylpyrrol -2-ylsulfanyl)-benzamide; N-[(1S,2R,4R)-7-azabicyclo[2.2.1]hept-2-yl]-4-(5-chloropyrrol -2-ylsulfanyl)-benzamide; N-[(1S,2R,4R)-7-azabicyclo[2.2.1]hept-2-yl]-4-(5-methoxypyrrol-2-ylsulfanyl)-benzamide; N-[(1S,2R,4R)-7-azabicyclo[2.2.1]hept-2-yl]-4-(5-trifluoromethylpyrrol-2-ylsulfanyl)-benzamide; N-[(1S,2R,4R)-7-azabicyclo[2.2.1]hept-2-yl]-4-(5-acetylpyrrol -2-ylsulfanyl)-benzamide; N-[(1S,2R,4R)-7-azabicyclo[2.2.1]hept-2-yl]-4-(5-acetaminopyrrol -2-ylsulfanyl)-benzamide; N-[(1S,2R,4R)-7-azabicyclo[2.2.1]hept-2-yl]-4-(5-cyanopyrrol -2-ylsulfanyl)-benzamide; N-[(1S,2R,4R)-7-azabicyclo[2.2.1]hept-2-yl]-4-(5-morpholin-4-yl-pyrrol-2-ylsulfanyl)-benzamide; N-[(1S,2R,4R)-7-azabicyclo[2.2.1]hept-2-yl]-4-(3H-imidazol-4-ylsulfanyl)-benzamide; N-[(1S,2R,4R)-7-azabicyclo[2.2.1]hept-2-yl]-4-(2-methyl-3H-imidazol-4-ylsulfanyl)-benzamide; N-[(1S,2R,4R)-7-azabicyclo[2.2.1]hept-2-yl]-4-(2-chloro-3H-imidazol -4-ylsulfanyl)-benzamide; N-[(1S,2R,4R)-7-azabicyclo[2.2.1]hept-2-yl]-4-(2-methoxy-3H-imidazol -4-ylsulfanyl)-benzamide; N-[(1S,2R,4R)-7-azabicyclo[2.2.1]hept-2-yl]-4-(2-trifluoromethyl-3H-imidazol-4-ylsulfanyl)-benzamide; N-[(1S,2R,4R)-7-azabicyclo[2.2.1]hept-2-yl]-4-(2-acetyl-3H-imidazol-4-ylsulfanyl)-benzamide; N-[(1S,2R,4R)-7-azabicyclo[2.2.1]hept-2-yl]-4-(2-acetamino-3H-imidazol -4-ylsulfanyl)-benzamide; N-[(1S,2R,4R)-7-azabicyclo[2.2.1]hept-2-yl]-4-(2-cyano -3H-imidazol-4-ylsulfanyl)-benzamide; N-[(1S,2R,4R)-7-azabicyclo[2.2.1]hept-2-yl]-4-(2-morpholin-4-yl-3H-imidazol-4-ylsulfanyl)-benzamide; N-[(1S,2R,4R) -7-azabicyclo[2.2.1]hept-2-yl]-4-(isoxazol-2-ylsulfanyl)-benzamide; N-[(1S,2R,4R)-7-azabicyclo[2.2.1]hept-2-yl]-4-(5-methylisoxazol-3-ylsulfanyl)-benzamide; N-[(1S,2R,4R)-7-azabicyclo[2.2.1]hept-2-yl]-4-(5-chloroisoxazol-3-ylsulfanyl)-benzamide; N-[(1S,2R,4R)-7-azabicyclo[2.2.1]hept-2-yl]-4-(5-methoxyisoxazol -3-ylsulfanyl)-benzamide; N-[(1S,2R,4R)-7-azabicyclo[2.2.1]hept-2-yl]-4-(5-trifluoromethylisoxazol -3-ylsulfanyl)-benzamide; N-[(1S,2R,4R)-7-azabicyclo[2.2.1]hept-2-yl]-4-(5-acetylisoxazol-3-ylsulfanyl)-benzamide; N-[(1S,2R,4R)-7-azabicyclo[2.2.1]hept-2-yl]-4-(5-acetaminoisoxazol-3-ylsulfanyl)-benzamide; N-[(1S,2R,4R)-7-azabicyclo[2.2.1]hept-2-yl]-4-(5-cyanoisoxazol -3-ylsulfanyl)-benzamide; N-[(1S,2R,4R)-7-azabicyclo[2.2.1]hept-2-yl]-4-(5-morpholin-4-yl-isoxazol -3-ylsulfanyl)-benzamide; N-[(1S,2R,4R)-7-azabicyclo[2.2.1]hept-2-yl]-4-(isothiazol -2-ylsulfanyl)-benzamide; N-[(1S,2R,4R)-7-azabicyclo[2.2.1]hept-2-yl]-4-(5-methylisothiazol-3-ylsulfanyl)-benzamide; N-[(1S,2R,4R)-7-azabicyclo[2.2.1]hept-2-yl]-4-(5-chloroisothiazol-3-ylsulfanyl)-benzamide; N-[(1S,2R,4R)-7-azabicyclo[2.2.1]hept-2-yl]-4-(5-methoxyisothiazol-3-ylsulfanyl)-benzamide; N-[(1S,2R,4R)-7-azabicyclo[2.2.1]hept-2-yl]-4-(5-trifluoromethylisothiazol -3-ylsulfanyl)-benzamide; N-[(1S,2R,4R)-7-azabicyclo[2.2.1]hept-2-yl]-4-(5-acetylisothiazol-3-ylsulfanyl)-benzamide; N-[(1S, 2R,4R)-7-azabicyclo[2.2.1]hept-2-yl]-4-(5-acetaminoisothiazol-3-ylsulfanyl)-benzamide; N-[(1S,2R,4R)-7-azabicyclo[2.2.1]hept-2-yl]-4-(5-cyanoisothiazol -3-ylsulfanyl)-benzamide; N-[(1S,2R,4R)-7-azabicyclo[2.2.1]hept-2-yl]-4-(5-morpholin -4-yl-isothiazol-3-ylsulfanyl)-benzamide; N-[(1S,2R,4R)-7-azabicyclo[2.2.1]hept-2-yl]-4-(6-methylpyridin-3-yloxy)-benzamide; N-[(1S,2R,4R)-7-azabicyclo[2.2.1]hept-2-yl]-4-(6-chloropyridin-3-yloxy)-benzamide; N-[(1S,2R,4R)-7-azabicyclo[2.2.1]hept-2-yl]-4-(6-methoxypyridin-3-yloxy)-benzamide; N-[(1S,2R,4R)-7-azabicyclo[2.2.1]hept-2-yl]-4-(6-trifluoromethylpyridin -3-yloxy)-benzamide; N-[(1S,2R,4R)-7-azabicyclo[2.2.1]hept-2-yl]-4-(6-acetylpyridin -3-yloxy)-benzamide; N-[(1S,2R,4R)-7-azabicyclo[2.2.1]hept-2-yl]-4-(6-acetaminopyridin -3-yloxy)-benzamide; N-[(1S,2R,4R)-7-azabicyclo[2.2.1]hept-2-yl]-4-(6-cyanopyridin-3-yloxy)-benzamide; N-[(1S,2R,4R)-7-azabicyclo[2.2.1]hept -2-yl]-4-(6-morpholin-4-yl-pyridin-3-yloxy)-benzamide; N-[(1S,2R,4R)-7-azabicyclo[2.2.1]hept-2-yl]-4-(2-methylpyridin-4-yloxy)-benzamide; N-[(1S,2R,4R)-7-azabicyclo[2.2.1]hept-2-yl]-4-(2-chloropyridin-4-yloxy)-benzamide; N-[(1S,2R,4R)-7-azabicyclo[2.2.1]hept-2-yl]-4-(2-methoxypyridin-4-yloxy)-benzamide; N-[(1S,2R,4R)-7-azabicyclo[2.2.1]hept-2-yl]-4-(2-trifluoromethylpyridin -4-yloxy)-benzamide; N-[(1S,2R,4R)-7-azabicyclo[2.2.1]hept-2-yl]-4-(2-acetylpyridin -4-yloxy)-benzamide; N-[(1S,2R,4R)-7-azabicyclo[2.2.1]hept-2-yl]-4-(2-acetaminopyridin -4-yloxy)-benzamide; N-[(1S,2R,4R)-7-azabicyclo[2.2.1]hept-2-yl]-4-(2-cyanopyridin-4-yloxy)-benzamide; N-[(1S,2R,4R)-7-azabicyclo[2.2.1]hept -2-yl]-4-(2-morpholin-4-yl-pyridin-4-yloxy)-benzamide; N-[(1S,2R,4R)-7-azabicyclo[2.2.1]hept-2-yl]-4-(5-methylpyridin-2-yloxy)-benzamide; N-[(1S,2R,4R)-7-azabicyclo[2.2.1]hept-2-yl]-4-(5-chloropyridin-2-yloxy)-benzamide; N-[(1S,2R,4R)-7-azabicyclo[2.2.1]hept-2-yl]-4-(5-methoxypyridin-2-yloxy)-benzamide; N-[(1S,2R,4R)-7-azabicyclo[2.2.1]hept-2-yl]-4-(5-trifluoromethylpyridin -2-yloxy)-benzamide; N-[(1S,2R,4R)-7-azabicyclo[2.2.1]hept-2-yl]-4-(5-acetylpyridin -2-yloxy)-benzamide; N-[(1S,2R,4R)-7-azabicyclo[2.2.1]hept-2-yl]-4-(5-acetaminopyridin -2-yloxy)-benzamide; N-[(1S,2R,4R)-7-azabicyclo[2.2.1]hept-2-yl]-4-(5-cyanopyridin-2-yloxy)-benzamide; N-[(1S,2R,4R)-7-azabicyclo[2.2.1]hept-2-yl]-4-(5-morpholin-4-yl-pyridin-2-yloxy)-benzamide; N-[(1S,2R,4R)-7-azabicyclo[2.2.1]hept-2-yl]-4-(4-methylpyridin-2-yloxy)-benzamide; N-[(1S,2R,4R)-7-azabicyclo[2.2.1]hept-2-yl]-4-(4-chloropyridin-2-yloxy)-benzamide; N-[(1S, 2R,4R)-7-azabicyclo[2.2.1]hept-2-yl]-4-(4-methoxypyridin-2-yloxy)-benzamide; N-[(1S,2R,4R)-7-azabicyclo[2.2.1]hept-2-yl]-4-(4-trifluoromethylpyridin -2-yloxy)-benzamide; N-[(1S,2R,4R)-7-azabicyclo[2.2.1]hept-2-yl]-4-(4-acetylpyridin -2-yloxy)-benzamide; N-[(1S,2R,4R)-7-azabicyclo[2.2.1]hept-2-yl]-4-(4-acetaminopyridin -2-yloxy)-benzamide; N-[(1S,2R,4R)-7-azabicyclo[2.2.1]hept-2-yl]-4-(4-cyanopyridin-2-yloxy)-benzamide; N-[(1S,2R,4R)-7-azabicyclo[2.2.1]hept -2-yl]-4-(4-morpholin-4-yl-pyridin-2-yloxy)-benzamide; N-[(1S,2R,4R)-7-azabicyclo[2.2.1]hept-2-yl]-4-(6-methylpyridin-2-yloxy)-benzamide; N-[(1S,2R,4R)-7-azabicyclo[2.2.1]hept-2-yl]-4-(6-chloropyridin-2-yloxy)-benzamide; N-[(1S,2R,4R)-7-azabicyclo[2.2.1]hept-2-yl]-4-(6-methoxypyridin-2-yloxy)-benzamide; N-[(1S,2R,4R)-7-azabicyclo[2.2.1]hept-2-yl]-4-(6-trifluoromethylpyridin -2-yloxy)-benzamide; N-[(1S,2R,4R)-7-azabicyclo[2.2.1]hept-2-yl]-4-(6-acetylpyridin -2-yloxy)-benzamide; N-[(1S,2R,4R)-7-azabicyclo[2.2.1]hept-2-yl]-4-(6-acetaminopyridin -2-yloxy)-benzamide; N-[(1S,2R,4R)-7-azabicyclo[2.2.1]hept-2-yl]-4-(6-cyanopyridin-2-yloxy)-benzamide; N-[(1S,2R,4R)-7-azabicyclo[2.2.1]hept -2-yl]-4-(6-morpholin-4-yl-pyridin-2-yloxy)-benzamide; N-[(1S,2R,4R)-7-azabicyclo[2.2.1]hept-2-yl]-4-(5-methylpyridin-3-yloxy)-benzamide; N-[(1S,2R,4R)-7-azabicyclo[2.2.1]hept-2-yl]-4-(5-chloropyridin-3-yloxy)-benzamide; N-[(1S,2R,4R)-7-azabicyclo[2.2.1]hept-2-yl]-4-(5-methoxypyridin-3-yloxy)-benzamide; N-[(1S,2R,4R)-7-azabicyclo[2.2.1]hept-2-yl]-4-(5-trifluoromethylpyridin -3-yloxy)-benzamide; N-[(1S,2R,4R)-7-azabicyclo[2.2.1]hept-2-yl]-4-(5-acetylpyridin -3-yloxy)-benzamide; N-[(1S,2R,4R)-7-azabicyclo[2.2.1]hept-2-yl]-4-(5-acetaminopyridin -3-yloxy)-benzamide; N-[(1S,2R,4R)-7-azabicyclo[2.2.1]hept-2-yl]-4-(5-cyanopyridin-3-yloxy)-benzamide; N-[(1S,2R,4R)-7-azabicyclo[2.2.1]hept-2-yl]-4-(5-morpholin-4-yl-pyridin-3-yloxy)-benzamide; N-[(1S,2R,4R)-7-azabicyclo[2.2.1]hept-2-yl]-4-(2-chloropyridin-3-yloxy)-benzamide; N-[(1S,2R,4R) -7-azabicyclo[2.2.1]hept-2-yl]-4-(4-chloropyridin-3-yloxy)-benzamide; N-[(1S,2R,4R)-7-azabicyclo[2.2.1]hept-2-yl]-4-(3-chloropyridin-4-yloxy)-benzamide; N-[(1S,2R,4R)-7-azabicyclo[2.2.1]hept-2-yl]-4-(3-chloropyridin-2-yloxy)-benzamide; N-[(1S,2R,4R)-7-azabicyclo[2.2.1]hept-2-yl]-4-(6-methylpyridin -3-ylsulfanyl)-benzamide; N-[(1S,2R,4R)-7-azabicyclo[2.2.1]hept-2-yl]-4-(6-chloropyridin -3-ylsulfanyl)-benzamide; N-[(1S,2R,4R)-7-azabicyclo[2.2.1]hept-2-yl]-4-(6-methoxypyridin -3-ylsulfanyl)-benzamide; N-[(1S,2R,4R)-7-azabicyclo[2.2.1]hept-2-yl]-4-(6-trifluoromethylpyridin-3-ylsulfanyl)-benzamide; N-[(1S,2R,4R)-7-azabicyclo[2.2.1]hept-2-yl]-4-(6-acetylpyridin-3-ylsulfanyl)-benzamide; N-[(1S,2R,4R)-7-azabicyclo[2.2.1]hept-2-yl]-4-(6-acetaminopyridin-3-ylsulfanyl)-benzamide; N-[(1S,2R,4R)-7-azabicyclo[2.2.1]hept-2-yl]-4-(6-cyanopyridin -3-ylsulfanyl)-benzamide; N-[(1S,2R,4R)-7-azabicyclo[2.2.1]hept-2-yl]-4-(6-morpholin-4-yl-pyridin -3-ylsulfanyl)-benzamide; N-[(1S,2R,4R)-7-azabicyclo[2.2.1]hept-2-yl]-4-(2-methylpyridin-4-ylsulfanyl)-benzamide; N-[(1S,2R,4R)-7-azabicyclo[2.2.1]hept-2-yl]-4-(2-chloropyridin-4-ylsulfanyl)-benzamide; N-[(1S,2R,4R)-7-azabicyclo[2.2.1]hept-2-yl]-4-(2-methoxypyridin-4-ylsulfanyl)-benzamide; N-[(1S,2R,4R)-7-azabicyclo[2.2.1]hept-2-yl]-4-(2-trifluoromethylpyridin -4-ylsulfanyl)-benzamide; N-[(1S,2R,4R)-7-azabicyclo[2.2.1]hept-2-yl]-4-(2-acetylpyridin -4-ylsulfanyl)-benzamide; N-[(1S,2R,4R)-7-azabicyclo[2.2.1]hept-2-yl]-4-(2-acetaminopyridin -4-ylsulfanyl)-benzamide; N-[(1S,2R,4R)-7-azabicyclo[2.2.1]hept -2-yl]-4-(2-cyanopyridin-4-ylsulfanyl)-benzamide; N-[(1S,2R,4R)-7-azabicyclo[2.2.1]hept-2-yl]-4-(2-morpholin-4-yl-pyridin-4-ylsulfanyl)-benzamide; N-[(1S,2R,4R)-7-azabicyclo[2.2.1]hept-2-yl]-4-(5-methylpyridin -2-ylsulfanyl)-benzamide; N-[(1S,2R,4R)-7-azabicyclo[2.2.1]hept-2-yl]-4-(5-chloropyridin -2-ylsulfanyl)-benzamide; N-[(1S,2R,4R)-7-azabicyclo[2.2.1]hept-2-yl]-4-(5-methoxypyridin -2-ylsulfanyl)-benzamide; N-[(1S,2R,4R)-7-azabicyclo[2.2.1]hept-2-yl]-4-(5-trifluoromethylpyridin-2-ylsulfanyl)-benzamide; N-[(1S,2R,4R)-7-azabicyclo[2.2.1]hept-2-yl]-4-(5-acetylpyridin-2-ylsulfanyl)-benzamide; N-[(1S 2R, 4R)-7-azabicyclo[2.2.1]hept-2-yl]-4-(5-acetaminopyridin-2-ylsulfanyl)-benzamide; N-[(1S,2R,4R)-7-azabicyclo[2.2.1]hept-2-yl]-4-(5-cyanopyridin -2-ylsulfanyl)-benzamide; N-[(1S,2R,4R)-7-azabicyclo[2.2.1]hept-2-yl]-4-(5-morpholin-4-yl-pyridin -2-ylsulfanyl)-benzamide; N-[(1S,2R,4R)-7-azabicyclo[2.2.1]hept-2-yl]-4-(4-methylpyridin -2-ylsulfanyl)-benzamide; N-[(1S,2R,4R)-7-azabicyclo[2.2.1]hept-2-yl]-4-(4-chloropyridin-2-ylsulfanyl)-benzamide; N-[(1S,2R,4R)-7-azabicyclo[2.2.1]hept-2-yl]-4-(4-methoxypyridin-2-ylsulfanyl)-benzamide; N-[(1S,2R,4R)-7-azabicyclo[2.2.1]hept-2-yl]-4-(4-trifluoromethylpyridin -2-ylsulfanyl)-benzamide; N-[(1S,2R,4R)-7-azabicyclo[2.2.1]hept-2-yl]-4-(4-acetylpyridin -2-ylsulfanyl)-benzamide; N-[(1S,2R,4R)-7-azabicyclo[2.2.1]hept-2-yl]-4-(4-acetaminopyridin -2-ylsulfanyl)-benzamide; N-[(1S,2R,4R)-7-azabicyclo[2.2.1]hept -2-yl]-4-(4-cyanopyridin-2-ylsulfanyl)-benzamide; N-[(1S,2R,4R)-7-azabicyclo[2.2.1]hept-2-yl]-4-(4-morpholin-4-yl-pyridin-2-ylsulfanyl)-benzamide; N-[(1S,2R,4R)-7-azabicyclo[2.2.1]hept-2-yl]-4-(6-methylpyridin -2-ylsulfanyl)-benzamide; N-[(1S,2R,4R)-7-azabicyclo[2.2.1]hept-2-yl]-4-(6-chloropyridin -2-ylsulfanyl)-benzamide; N-[(1S,2R,4R)-7-azabicyclo[2.2.1]hept-2-yl]-4-(6-methoxypyridin -2-ylsulfanyl)-benzamide; N-[(1S,2R,4R)-7-azabicyclo[2.2.1]hept-2-yl]-4-(6-trifluoromethylpyridin-2-ylsulfanyl)-benzamide; N-[(1S,2R,4R)-7-azabicyclo[2.2.1]hept-2-yl]-4-(6-acetylpyridin-2-ylsulfanyl)-benzamide; N-[(1S,2R,4R)-7-azabicyclo[2.2.1]hept-2-yl]-4-(6-acetaminopyridin-2-ylsulfanyl)-benzamide; N-[(1S,2R,4R)-7-azabicyclo[2.2.1]hept-2-yl]-4-(6-cyanopyridin -2-ylsulfanyl)-benzamide; N-[(1S,2R,4R)-7-azabicyclo[2.2.1]hept-2-yl]-4-(6-morpholin-4-yl-pyridin -2-ylsulfanyl)-benzamide; N-[(1S,2R,4R)-7-azabicyclo[2.2.1]hept-2-yl]-4-(5-methylpyridin -3-ylsulfanyl)-benzamide; N-[(1S,2R,4R)-7-azabicyclo[2.2.1]hept-2-yl]-4-(5-chloropyridin-3-ylsulfanyl)-benzamide; N-[(1S,2R,4R)-7-azabicyclo[2.2.1]hept-2-yl]-4-(5-methoxypyridin-3-ylsulfanyl)-benzamide; N-[(1S,2R,4R)-7-azabicyclo[2.2.1]hept-2-yl]-4-(5-trifluoromethylpyridin -3-ylsulfanyl)-benzamide; N-[(1S,2R,4R)-7-azabicyclo[2.2.1]hept-2-yl]-4-(5-acetylpyridin -3-ylsulfanyl)-benzamide; N-[(1S,2R,4R)-7-azabicyclo[2.2.1]hept-2-yl]-4-(5-acetaminopyridin -3-ylsulfanyl)-benzamide; N-[(1S,2R,4R)-7-azabicyclo[2.2.1]hept-2-yl]-4-(5-cyanopyridin-3-ylsulfanyl)-benzamide; N-[(1S,2R,4R)-7-azabicyclo[2.2.1]hept-2-yl]-4-(5-morpholin-4-yl-pyridin-3-ylsulfanyl)-benzamide; N-[(1S,2R,4R)-7-azabicyclo[2.2.1]hept-2-yl]-4-(2-chloropyridin -3-ylsulfanyl)-benzamide; N-[(1S,2R,4R)-7-azabicyclo[2.2.1]hept-2-yl]-4-(4-chloropyridin -3-ylsulfanyl)-benzamide; N-[(1S,2R,4R)-7-azabicyclo[2.2.1]hept-2-yl]-4-(3-chloropyridin -4-ylsulfanyl)-benzamide; N-[(1S,2R,4R)-7-azabicyclo[2.2.1]hept-2-yl]-4-(3-chloropyridin-2-ylsulfanyl)-benzamide; or a pharmaceutically acceptable salt thereof.

14. The compound of claim 10, wherein aryl is naphthyl or substituted naphthyl.

15. The compound according to claim 14, wherein the compound is N-[(1S,2R,4R)-7-azabicyclo[2.2.1]hept-2-yl]-2-naphthamide; N-[(1S,2R,4R)-7-azabicyclo[2.2.1]hept-2-yl]-7-methoxy-2-naphthamide; or a pharmaceutically acceptable salt thereof.

16. The compound according to claim 14, wherein the compound is N-[(1S,2R,4R)-7-azabicyclo[2.2.1]hept-2-yl]-7-methyl-2-naphthamide; N-[(1S, 2R, 4R)-7-azabicyclo[2.2.1]hept-2-yl]-7-hydroxy-2-naphthamide; N-[(1S,2R,4R) -7-azabicyclo[2.2.1]hept-2-yl]-7-methoxy-2-naphthamide; N-[(1S,2R,4R)-7-azabicyclo[2.2.1]hept-2-yl]-7-mercapto-2-naphthamide; N-[(1S,2R,4R)-7-azabicyclo[2.2.1]hept-2-yl]-7-methylthio-2-naphthamide; 7-amino-N-[(1S,2R,4R) -7-azabicyclo[2.2.1]hept-2-yl]-2-naphthamide; N-[(1S,2R,4R)-7-azabicyclo[2.2.1]hept-2-yl]-7-methylamino-2-naphthamide; N-[(1S,2R,4R)-7-azabicyclo[2.2.1]hept-2-yl]-7-fluoro-2-naphthamide; N-[(1S,2R,4R)-7-azabicyclo[2.2.1]hept-2-yl]-7-cyano-2-naphthamide; N-[(1S,2R,4R)-7-azabicyclo[2.2.1]hept-2-yl]-7-chloro-2-naphthamide; N-[(1S,2R,4R)-7-azabicyclo[2.2.1]hept-2-yl]-7-bromo-2-naphthamide; N-[(1S,2R,4R)-7-azabicyclo[2.2.1]hept-2-yl]-7-iodo-2-naphthamide; N-[(1S,2R,4R)-7-azabicyclo[2.2.1]hept-2-yl]-7-nitro-2-naphthamide; N-[(1S,2R,4R)-7-azabicyclo[2.2.1]hept-2-yl]-7-ethynyl-2-naphthamide; N-[(1S,2R,4R)-7-azabicyclo[2.2.1]hept-2-yl]-7-trifluoromethyl-2-naphthamide; N-[(1S,2R,4R)-7-azabicyclo[2.2.1]hept-2-yl]-7-prop-1-ynyl-2-naphthamide; N-[(1S,2R,4R)-7-azabicyclo[2.2.1]hept-2-yl]-7-ethenyl-2-naphthamide; N-[(1S,2R,4R)-7-azabicyclo[2.2.1]hept-2-yl]-7-(3-hydroxyprop-1-ynyl)-2-naphthamide; 7-(acetylamino)-N-[(1S,2R,4R)-7-azabicyclo[2.2.1]hept-2-yl]-2-naphthamide; N-[(1S,2R,4R)-7-azabicyclo[2.2.]hept-2-yl]-7-(formylamino)-2-naphthamide; N-[(1S,2R,4R)-7-azabicyclo[2.2.1]hept-2-yl]-5-methyl-2-naphthamide; N-[(1S,2R,4R) -7-azabicyclo[2.2.1]hept-2-yl]-5-hydroxy-2-naphthamide; N-[(1S,2R,4R)-7-azabicyclo[2.2.1]hept-2-yl]-5-methoxy-2-naphthamide; N-[(1S,2R,4R)-7-azabicyclo[2.2.1]hept-2-yl]-5-mercapto-2-naphthamide; N-[(1S,2R,4R)-7-azabicyclo[2.2.1]hept-2-yl]-5-methylthio-2-naphthamide; 5-Amino-N-[(1S,2R,4R) -7-azabicyclo[2.2.1]hept-2-yl]-2-naphthamide; N-[(1S,2R,4R)-7-azabicyclo[2.2.1]hept-2-yl]-5-methylamino-2-naphthamide; N-[(1S,2R,4R)-7-azabicyclo[2.2.1]hept-2-yl]-5-fluoro-2-naphthamide; N-[(1S,2R,4R)-7-azabicyclo[2.2.1]hept-2-yl]-5-cyano-2-naphthamide; N-[(1S,2R,4R)-7-azabicyclo[2.2.1]hept-2-yl]-5-chloro-2-naphthamide; N-[(1S,2R,4R)-7-azabicyclo[2.2.1]hept-2-yl]-5-bromo-2-naphthamide; N-[(1S,2R,4R)-7-azabicyclo[2.2.1]hept-2-yl]-5-iodo-2-naphthamide; N-[(1S,2R,4R)-7-azabicyclo[2.2.1]hept-2-yl]-5-nitro-2-naphthamide; N-[(1S,2R,4R)-7-azabicyclo[2.2.1]hept-2-yl]-5-ethynyl-2-naphthamide; N-[(1S,2R,4R)-7-azabicyclo[2.2.1]hept-2-yl]-5-trifluoromethyl-2-naphthamide; N-[(1S,2R,4R)-7-azabicyclo[2.2.1]hept-2-yl]-5-prop-1-ynyl-2-naphthamide; N-[(1S,2R,4R)-7-azabicyclo[2.2.1]hept-2-yl]-5-ethenyl-2-naphthamide; N-[(1S,2R,4R)-7-azabicyclo[2.2.1]hept-2-yl]-5-(3-hydroxyprop-1-ynyl)-2-naphthamide; 5-(acetylamino)-N-[(1S,2R,4R)-7-azabicyclo[2.2.1]hept-2-yl]-2-naphthamide; N-[(1S,2R,4R)-7-azabicyclo[2.2.1]hept-2-yl]-5-(formylamino)-2-naphthamide; N-[(1S,2R,4R)-7-azabicyclo[2.2.1]hept-2-yl]-8-methyl-2-naphthamide; N-[(1S,2R,4R) -7-azabicyclo[2.2.1]hept-2-yl]-8-hydroxy-2-naphthamide; N-[(1S,2R,4R)-7-azabicyclo[2.2.1]hept-2-yl]-8-methoxy-2-naphthamide; N-[(1S,2R,4R)-7-azabicyclo[2.2.1]hept-2-yl]-8-mercapto-2-naphthamide; N-[(1S,2R,4R)-7-azabicyclo[2.2.1]hept-2-yl]-8-methylthio-2-naphthamide; 8-Amino-N-[(1S,2R,4R) -7-azabicyclo[2.2.1]hept-2-yl]-2-naphthamide; N-[(1S,2R,4R)-7-azabicyclo[2.2.1]hept-2-yl]-8-methylamino-2-naphthamide; N-[(1S,2R,4R)-7-azabicyclo[2.2.1]hept-2-yl]-8-fluoro-2-naphthamide; N-[(1S,2R,4R)-7-azabicyclo[2.2.1]hept-2-yl]-8-cyano-2-naphthamide; N-[(1S,2R,4R)-7-azabicyclo[2.2.1]hept-2-yl]-8-chloro-2-naphthamide; N-[(1S,2R,4R)-7-azabicyclo[2.2.1]hept-2-yl]-8-bromo-2-naphthamide; N-[(1S,2R,4R)-7-azabicyclo[2.2.1]hept-2-yl]-8-iodo-2-naphthamide; N-[(1S,2R,4R)-7-azabicyclo[2.2.1]hept-2-yl]-8-nitro-2-naphthamide; N-[(1S,2R,4R)-7-azabicyclo[2.2.1]hept-2-yl]-8-ethynyl-2-naphthamide; N-[(1S,2R,4R)-7-azabicyclo[2.2.1]hept-2-yl]-8-trifluoromethyl-2-naphthamide; N-[(1S,2R,4R)-7-azabicyclo[2.2.1]hept-2-yl]-8-prop-1-ynyl-2-naphthamide; N-[(1S,2R,4R)-7-azabicyclo[2.2.1]hept-2-yl]-8-ethenyl-2-naphthamide; N-[(1S,2R,4R)-7-azabicyclo[2.2.1]hept-2-yl]-8-(3-hydroxyprop-1-ynyl)-2-naphthamide; 8-(acetylamino)-N-[(1S,2R,4R)-7-azabicyclo[2.2.1]hept-2-yl]-2-naphthamide; N-[(1S,2R,4R)-7-azabicyclo[2.2.1]hept-2-yl]-8-(formylamino)-2-naphthamide; N-[(1S,2R,4R)-7-azabicyclo[2.2.1]hept-2-yl]-7-carbamoyl-2-naphthamide; N-[(1S,2R,4R) -7-azabicyclo[2.2.1]hept-2-yl]-5-carbamoyl-2-naphthamide; N-[(1S,2R,4R)-7-azabicyclo[2.2.1]hept-2-yl]-8-carbamoyl-2-naphthamide; or pharmaceutically acceptable salt thereof.

17. The compound according to claim 3, wherein Q is formula II.

18. The compound of claim 17, wherein each R₄ is independently H, lower alkyl, or substituted lower alkyl.

19. The compound according to claim 18, wherein R₆ is an amino protecting group.

20. The compound according to claim 19, wherein R₆ is H, or lower alkyl optionally substituted with up to 3 substituents independently selected from F, Cl, Br, I, —OH, —CN, —NH₂, —NH(alkyl), or —N(alkyl)₂.

21. The compound of claim 20, wherein R₁ is H or lower alkyl, and wherein R₂ is H or lower alkyl.

22. The compound of claim 21, wherein at least one R₄ is H and one R₄ is H or lower alkyl optionally substituted with 1 substituent selected from —CN, —NO₂, —OR₁₀, —SR₁₀, —S(O)R₁₀, —S(O)₂R₁₀, —OS(O)₂R₁₀, —NR₁₀R₁₀, —C(O)R₁₀, —C(O)OR₁₀, —C(S)R₁₀, —C(O)NR₁₀R₁₀, —NR₁₀C(O)R₁₀, —NR₁₀C(O)NR₁₀R₁₀, —S(O)₂NR₁₀R₁₀, —NR₁₀S(O)₂R₁₀, or phenyl optionally substituted with up to 4 substitutents independently selected from F, Cl, Br, I, R₁₃, and R₁₅, provided that when said lower alkyl is optionally substituted, said lower alkyl can be fuirther optionally substituted with up to 3 substituents independently selected from F, Cl, Br, and I, and further provided that R₁₀ is H, lower alkyl, or halogenated lower alkyl.

23. The compound according to claim 22, wherein R₁, R₂, and each R₄ are H.

24. The compound according to claim 23, wherein the compound is N-((1S,2R,4R)-7-azabicyclo[2.2.1]hept-2-yl)indane-5-carboxamide; or a pharmaceutically acceptable salt thereof.

25. A compound of formula A-L-B or a pharmaceutically acceptable salt thereof, wherein A is a 7-azabicyclo[2.2.1]heptane ring having 1S, 2R, and 4R stereochemistry; L is a linking moiety,
wherein the linking moiety is bonded to the C-2 carbon of the heptane ring in an exo orientation; and
wherein B is phenyl, naphthyl, or phenyl fused to a 5- or 6-membered saturated or partially unsaturated ring, all optionally substituted with up to 4 substituents where valency allows with alkyl, alkenyl, alkynyl, cycloalkyl, heterocycloalkyl, halogenated alkyl, halogenated alkenyl, halogenated alkynyl, halogenated cycloalkyl, halogenated heterocycloalkyl, substituted alkyl, substituted alkenyl, substituted alkynyl, substituted cycloalkyl, substituted heterocycloalkyl, lactam heterocycloalkyl, R₇, R₉, —NO₂, —CN, F, Cl, Br, I, —OR₈, —SR₈, —S(O)₂R₈, —S(O)R₈, —OS(O)₂R₈, —N(R₈)₂, —C(O)R₈, —C(S)R₈, —C(O)OR₈, —C(O)N(R₈)₂, —NR₈C(O)R₈, —S(O)₂N(R₈)₂, —NR₈S(O)₂R₈, —N(R₈)C(O)N(R₈)₂, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅, or optionally a carbon atom is substituted with ═O or ═S where valency allows; wherein
R₇ is 5-membered heteroaromatic mono-cyclic moieties containing within the ring 1-3 heteroatoms independently selected from the group consisting of ═N—, —N(R₂₀)—, —O—, and —S—, and having 0-1 substituent selected from R₁₇ and further having 0-3 substituents independently selected from F, Cl, Br, or I, or R₇ is 9-membered fused-ring moieties having a 6-membered ring fused to a 5-membered ring including the formula 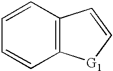
wherein G₁ is O, S or NR₂₀, 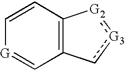
wherein G is C(R₁₄) or N, and each G₂ and G₃ are independently selected from C(R₁₄)₂, C(R₁₄), O, S, N, and N(R₂₀), provided that both G₂ and G₃ are not simultaneously O or S, or 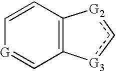
wherein G is C(R₁₄) or N, and each G₂ and G₃ are independently selected from C(R₁₄)₂, C(R₁₄), O, S, N, and N(R₂₀), each 9-membered bicyclic ring having 0-1 substituent selected from R₁₇ and 0-3 substituents independently selected from F, Cl, Br, or I, wherein the R₇ moiety attaches to other substituents as defined in formula I at any position on either ring as valency allows;
Each R₈ is independently H, alkyl, halogenated alkyl, substituted alkyl, cycloalkyl, halogenated cycloalkyl, substituted cycloalkyl, heterocycloalkyl, halogenated heterocycloalkyl, substituted heterocycloalkyl, R₇, R₉, phenyl optionally substituted with 1-4 substituents independently selected from F, Cl, Br, I, R₁₃, and R₁₅, or naphthyl optionally substituted with 1-4 substituents independently selected from F, Cl, Br, I, R₁₃, and R₁₅;
R₉ is 6-membered heteroaromatic mono-cyclic moieties containing within the ring 1-3 heteroatoms selected from ═N— and having 0-1 substituent selected from R₁₇ and 0-3 substituent(s) independently selected from F, Cl, Br, or I, or R₉ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₁₇ and 0-3 substituent(s) independently selected from F, Cl, Br, or I, wherein the R₉ moiety attaches to other substituents as defined in formula I at any position on either ring as valency allows;
Each R₁₁ is independently H, alkyl, cycloalkyl, heterocycloalkyl, halogenated alkyl, halogenated cycloalkyl, or halogenated heterocycloalkyl;
R₁₂ is alkyl, cycloalkyl, heterocycloalkyl, halogenated alkyl, halogenated cycloalkyl, halogenated heterocycloalkyl, substituted alkyl, substituted cycloalkyl, substituted heterocycloalkyl, —OR₁₁, —SR₁₁, —S(O)R₁₁, —S(O)₂R₁₁, —OS(O)₂R₁₁, —NR₁₁R₁₁, —C(O)R₁₁, —C(S)R₁₁, —C(O)OR₁₁, —NO₂, —CN, —C(O)N(R₁₁)₂, —NR₁₁C(O)R₁₁, —NR₁₁C(O)N(R₁₁)₂, —S(O)₂N(R₁₁)₂, or —NR₁₁S(O)₂R₁₁;
R₁₃ is —OR₁₁, —SR₁₁, —SOR₁₁, —SO₂R₁₁, —OSO₂R₁₁, —N(R₁₁)₂, —C(O)R₁₁, —C(O)OR₁₁, —C(S)R₁₁, —C(O)N(R₁₁)₂, —NO₂—CN, —CF₃, —NR₁₁C(O)R₁₁, —NR₁₁C(O)N(R₁₁)₂, —S(O)₂N(R₁₁)₂, or —NR₁₁S(O)₂R₁₁;
R₁₄ is H or R₁₉;
R₁₅ is lactam heterocycloalkyl, R₇, R₉, or R₁₉;
Each R₁₆ is independently H, alkyl, cycloalkyl, halogenated alkyl, or halogenated cycloalkyl;
R₁₉ is alkyl, cycloalkyl, heterocycloalkyl, phenyl, or naphthyl, each optionally substituted with 1-4 substituents independently selected from F, Cl, Br, I, —OR₁₆, —SR₁₆, —S(O)₂R₁₆, —S(O)R₁₆, —OS(O)₂R₁₆, —N(R₁₆)₂, —C(O)R₁₆, —C(S)R₁₆, —C(O)OR₁₆, —NO₂, —C(O)N(R₁₆)₂, —CN, —NR₁₆C(O)R₁₆, —NR₁₆C(O)N(R₁₆)₂, —S(O)₂N(R₁₆)₂, or —NR₁₆S(O)₂R₁₆, and the cycloalkyl and heterocycloalkyl also being further optionally substituted with ═O or ═S; and
R₂₀ is H, alkyl, halogenated alkyl, substituted alkyl, cycloalkyl, halogenated cycloalkyl, substituted cycloalkyl, phenyl, —SO₂R₈, or phenyl having 1 substituent selected from R₁₂ and further having 0-3 substituents independently selected from F, Cl, Br, or I.

26. A pharmaceutical composition comprising a compound according to claim 1. an anti-psychotic agent, and a pharmaceutically acceptable excipient.

27. The pharmaceutical composition according to claim 26, wherein said compound and said agent are to be independently administered rectally, topically, orally, sublingually, or parenterally for a therapeutically effective interval.

28. The pharmaceutical composition according to claim 26, wherein said compound is administered in an amount of from about 0.001 to about 100 mg/kg of body weight of said mammal per day.

29. The pharmaceutical composition according to claim 26, wherein said compound is administered in an amount of from about 0.1 to about 50 mg/kg of body weight of said mammal per day.

30. The pharmaceutical composition according to claim 26, wherein the composition comprises a compound according to claim 1 and a pharmaceutically acceptable excipient.

31. The pharmaceutical composition according to claim 30, wherein said compound is administered rectally, topically, orally, sublingually, or parenterally for a therapeutically effective interval.

32. The pharmaceutical composition according to claim 30, wherein said compound is administered in an amount of from about 0.001 to about 100 mg/kg of body weight of said mammal per day.

33. The pharmaceutical composition according to claim 30, wherein said compound is administered in an amount of from about 0.1 to about 50 mg/kg of body weight of said mammal per day.

34. A method for treating a disease or condition in a mammal in need thereof, wherein the mammal would receive symptomatic relief from the administration of an α7 nicotinic acetylcholine receptor agonist comprising administering to the mammal a therapeutically effective amount of a compound according to claim 1.

35. The method according to claim 34, wherein the disease or condition is cognitive and attention deficit symptoms of Alzheimer's, neurodegeneration associated with diseases such as Alzheimer's disease, pre-senile dementia (mild cognitive impairment), or senile dementia.

36. The method according to claim 34, wherein the disease or condition is schizophrenia or psychosis.

37. The method of claim 36, wherein the mammal would receive symptomatic relief from the administration of a therapeutically effective amount of α7 nicotinic acetylcholine receptor agonist and an anti-psychotic agent for a therapeutically effective interval.

38. The use according to claim 34, wherein the disease or condition is depression, or anxiety and general anxiety disorders and post traumatic stress disorder.

39. The use according to claim 34, wherein the disease or condition is attention deficit disorder, or attention deficit hyperactivity disorder.

40. The method according to claim 34, wherein the disease or condition is mood and affective disorders, amyotrophic lateral sclerosis, borderline personality disorder, traumatic brain injury, behavioral and cognitive problems in general and associated with brain tumors, AIDS dementia complex, dementia associated with Down's syndrome, dementia associated with Lewy Bodies, Huntington's disease, Parkinson's disease, tardive dyskinesia, Pick's disease, dysregulation of food intake including bulemia and anorexia nervosa, withdrawal symptoms associated with smoking cessation and dependant drug cessation, Gilles de la Tourette's Syndrome, age-related macular degeneration, glaucoma, neurodegeneration associated with glaucoma, or symptoms associated with pain.
